(19) United States
(12) Patent Application Publication
(10) Pub. No.: US 2011/0046170 A1
(43) Pub. Date: Feb. 24, 2011

(54) NOVEL PYRIMIDINE-PYRIDINE DERIVATIVES

(76) Inventors: Martin Bolli, Allschwil (CH); Gyrille Lescop, Kembs (FR); Boris Mathys, Prattein (CH); Claus Mueller, Weil am Rhein (DE); Oliver Nayler, Arlesheim (CH); Beat Steiner, Dornach (CH)

Correspondence Address: HOXIE & ASSOCIATES LLC, 75 MAIN STREET , SUITE 301, MILLBURN, NJ, 07041, US

(21) Appl. No.: 12/920,572

(22) PCT Filed: Mar. 3, 2009
(86) PCT No.: PCT/IB2009/050848

§ 371 Date: Nov. 2, 2010

(30) Foreign Application Priority Data
Mar. 6, 2008 (IB) PCT/IB2008/050818

(51) Int. Cl. A61K 31/506; C07D 413/14; C07D 417/14; A61P 37/06; A61P 29/00; A61P 25/00; A61P 1/00; A61P 17/06; A61P 11/06; A61P 3/10; A61P 11/02; A61P 27/02; A61P 17/00; A61P 35/00; A61P 35/04

(52) U.S. Cl. 514/275; 544/331; 544/316

(57) ABSTRACT

The invention relates to novel pyrimidine-pyridine derivatives, their preparation and their use as pharmaceutically active compounds. Said compounds particularly act as immunomodulating agents.

### FIELD OF THE INVENTION

The present invention relates to S1P1/EDG1 receptor agonists of formula (I) and their use as active ingredients in the preparation of pharmaceutical compositions. The invention also concerns related aspects including processes for the preparation of the compounds, pharmaceutical compositions containing a compound of the formula (I), and their use as compounds improving vascular function and as immunomodulating agents, either alone or in combination with other active compounds or therapies.

### BACKGROUND OF THE INVENTION

The human immune system is designed to defend the body against foreign micro-organisms and substances that cause infection or disease. Complex regulatory mechanisms ensure that the immune response is targeted against the intruding substance or organism and not against the host. In some cases, these control mechanisms are unregulated and autoimmune responses can develop. A consequence of the uncontrolled inflammatory response is severe organ, cell, tissue or joint damage. With current treatment, the whole immune system is usually suppressed and the body's ability to react to infections is also severely compromised. Typical drugs in this class include azathioprine, chlorambucil, cyclophosphamide, cyclosporin, or methotrexate. Corticosteroids which reduce inflammation and suppress the immune response, may cause side effects when used in long term treatment. Nonsteroidal anti-inflammatory drugs (NSAIDs) can reduce pain and inflammation, however, they exhibit considerable side effects. Alternative treatments include agents that activate or block cytokine signaling.

Orally active compounds with immunomodulating properties, without compromising immune responses and with reduced side effects would significantly improve current treatments of uncontrolled inflammatory disease.

In the field of organ transplantation the host immune response must be suppressed to prevent organ rejection. Organ transplant recipients can experience some rejection even when they are taking immunosuppressive drugs. Rejection occurs most frequently in the first few weeks after transplantation, but rejection episodes can also happen months or even years after transplantation. Combinations of up to three or four medications are commonly used to give maximum protection against rejection while minimizing side effects. Current standard drugs used to treat the rejection of transplanted organs interfere with discrete intracellular pathways in the activation of T-type or B-type white blood cells. Examples of such drugs are cyclosporin, daclizumab, basiliximab, everolimus, or FK506, which interfere with cytokine release or signaling; azathioprine or leflunomide, which inhibit nucleotide synthesis; or 15-deoxyspergualin, an inhibitor of leukocyte differentiation.

The beneficial effects of broad immunosuppressive therapies relate to their effects; however, the generalized immunosuppression which these drugs produce diminishes the immune system's defense against infection and malignancies. Furthermore, standard immunosuppressive drugs are often used at high dosages and can cause or accelerate organ damage.

### DESCRIPTION OF THE INVENTION

The present invention provides novel compounds of formula (I) that are agonists for the G protein-coupled receptor S1P1/EDG1 and have a powerful and long-lasting immunomodulating effect which is achieved by reducing the number of circulating and infiltrating T- and B-lymphocytes, without affecting their maturation, memory, or expansion. The reduction of circulating T-/B-lymphocytes as a result of S1P1/EDG1 agonism, possibly in combination with the observed improvement of endothelial cell layer function associated with S1P1/EDG1 activation, makes such compounds useful to treat uncontrolled inflammatory disease and to improve vascular functionality.

The compounds of the present invention can be utilized alone or in combination with standard drugs inhibiting T-cell activation, to provide a new immunomodulating therapy with a reduced propensity for infections when compared to standard immunosuppressive therapy. Furthermore, the compounds of the present invention can be used in combination with reduced dosages of traditional immunosuppressant therapies, to provide on the one hand effective immunomodulating activity, while on the other hand reducing end organ damage associated with higher doses of standard immunosuppressive drugs. The observation of improved endothelial cell layer function associated with S1P1/EDG1 activation provides additional benefits of compounds to improve vascular function.

The nucleotide sequence and the amino acid sequence for the human S1P1/EDG1 receptor are known in the art and are published in e.g.: Hla, T., and Maciag, T. J. Biol. Chem. 265 (1990), 9308-9313; WO 91/15583 published 17 Oct. 1991; WO 99/46277 published 16 Sep. 1999. The potency and efficacy of the compounds of formula (I) are assessed using a GTPγS assay to determine EC₅₀ values and by measuring the circulating lymphocytes in the rat after oral administration, respectively (see in Examples).

i) The invention relates to novel pyrimidine-pyridine compounds of the formula (I),

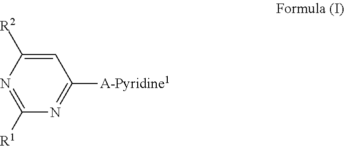

wherein
A represents

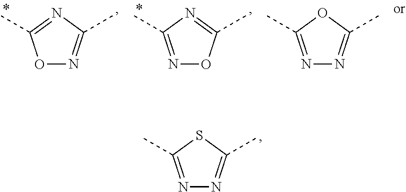

wherein the asterisks indicate the bond that is linked to the pyrimidine group of formula (I);
R¹ represents C₁₋₄-alkoxy, C₁₋₄-alkylamino, N—C₁₋₄-alkyl-N—C₁₋₃-alkylamino, C₃₋₅-cycloalkylamino, C₃₋₅-cycloalkylmethylamino, pyrrolidine, or piperidine;
R² represents C₁₋₂-alkyl, or C₃₋₄-alkyl; and
Pyridine¹ represents

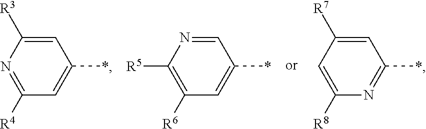

wherein the asterisks indicate the bond with which the pyridine ring is bound to the ring A of formula (I);
and in case R¹ represents C₁₋₄-alkylamino, N—C₁₋₄-alkyl-N—C₁₋₃-alkylamino, C₃₋₅-cycloalkylamino, or C₃₋₅-cycloalkylmethylamino, Pyridine¹ can also represent

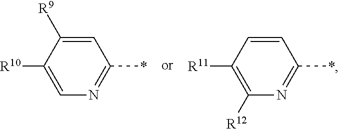

wherein the asterisks indicate the bond with which the pyridine ring is bound to the ring A of formula (I);
R³ represents C₁₋₄-alkyl, C₁₋₃-alkoxy, or NR^{3a}R^{3b};
R^{3a} represents C₁₋₂-alkyl;
R^{3b} represents hydrogen, or methyl; and
   R⁴ represents hydrogen, chloro, or C₁₋₂-alkyl;
   R⁵ represents C₁₋₄-alkyl, C₁₋₃-alkoxy, or NR^{5a}R^{5b};
R^{5a} represents C₁₋₃-alkyl;
R^{5b} represents hydrogen, or C₁₋₂-alkyl; and
   R⁶ represents C₁₋₂-alkyl;
   R⁷ represents C₁₋₄-alkyl, and R⁸ represents C₁₋₂-alkyl; or
   R⁷ represents C₁₋₂-alkyl, and R⁸ represents C₁₋₄-alkyl;
   R⁹ represents C₁₋₂-alkyl; and
   R¹⁰ represents C₁₋₄-alkyl;
   and
   R¹¹ represents C₁₋₄-alkyl; and
   R¹² represents C₁₋₂-alkyl.

The general terms used hereinbefore and hereinafter preferably have, within this disclosure, the following meanings, unless otherwise indicated:

The term C_{x-y}-alkyl, x and y being an integer, means saturated, branched or straight chain alkyl groups with x to y carbon atoms. Likewise, the term C₁₋₄-alkyl means saturated, branched or straight chain alkyl groups with one to four carbon atoms. Examples of C₁₋₄-alkyl groups are methyl, ethyl, n-propyl, iso-propyl, n-butyl, and iso-butyl (preferably methyl, ethyl, n-propyl, iso-propyl, or iso-butyl). Likewise, the term C_{1,3}-alkyl means saturated, branched or straight chain alkyl groups with one to three carbon atoms. Examples of C₁₋₃-alkyl groups are methyl, ethyl, n-propyl, and iso-propyl (preferably methyl, or ethyl).

The term C_{x-y}-alkoxy means a R-0 group, wherein R is a C_{x-y}-alkyl. Examples of C₁₋₄-alkoxy groups are methoxy, ethoxy, n-propoxy, iso-propoxy, and iso-butoxy.

The term C₃₋₅-cycloalkyl refers to a saturated cyclic hydrocarbon ring system with 3 to 5 carbon atoms, i.e. cyclopropyl, cyclobutyl, or cyclopentyl.

ii) Another embodiment of the invention relates to pyrimidine-pyridine derivatives of the formula (I) according to embodiment i), wherein Pyridine¹ represents

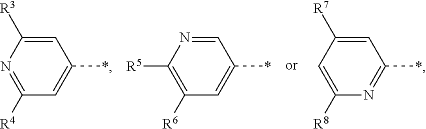

wherein the asterisks indicate the bond with which the pyridine ring is bound to the ring A of formula (I).
iii) Another embodiment of the invention relates to pyrimidine-pyridine derivatives of the formula (I) according to embodiment i) or ii), wherein A represents

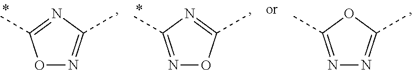

wherein the asterisks indicate the bond that is linked to the pyrimidine group of formula (I).
iv) Another embodiment of the invention relates to pyrimidine-pyridine derivatives of the formula (I) according to embodiment i) or ii), wherein A represents

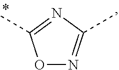

wherein the asterisk indicates the bond that is linked to the pyrimidine group of formula (I).
v) Another embodiment of the invention relates to pyrimidine-pyridine derivatives of the formula (I) according to embodiment i) or ii), wherein A represents

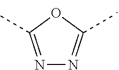

vi) Another embodiment of the invention relates to pyrimidine-pyridine derivatives according to any one of the embodiments i) to v), wherein R¹ represents C₁₋₄-alkylamino, or N—C₁₋₄-alkyl-N—C₁₋₃-alkylamino.
vii) Another embodiment of the invention relates to pyrimidine-pyridine derivatives according to any one of the embodiments i) to v), wherein R¹ represents C₁₋₄-alkylamino.
viii) Another embodiment of the invention relates to pyrimidine-pyridine derivatives according to any one of the embodiments i) to vii), wherein R² represents C₁₋₂-alkyl.
ix) Another embodiment of the invention relates to pyrimidine-pyridine derivatives according to any one of the embodiments i) to vii), wherein R² represents methyl.
x) Another embodiment of the invention relates to pyrimidine-pyridine derivatives according to any one of the embodiments i) to ix), wherein Pyridine¹ represents

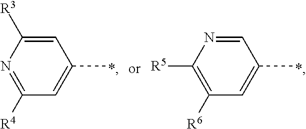

wherein the asterisks indicate the bond with which the pyridine ring is bound to the ring A of formula (I);
R³ represents C₁₋₄-alkyl, C₁₋₃-alkoxy, or NR^{3a}R^{3b};
R^{3a} represents C₁₋₂-alkyl;
R^{3b} represents hydrogen, or methyl; and
   R⁴ represents chloro or C₁₋₂-alkyl;
   and
   R⁵ represents C₁₋₄-alkyl, C₁₋₃-alkoxy, or NR^{5a}R^{5b};
R^{5a} represents C₁₋₃-alkyl;
R^{5b} represents hydrogen, or C₁₋₂-alkyl; and
   R⁶ represents C₁₋₂-alkyl.
   xi) Another embodiment of the invention relates to pyrimidine-pyridine derivatives according to any one of the embodiments i) to ix), wherein Pyridine¹ represents

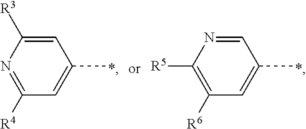

wherein the asterisks indicate the bond with which the pyridine ring is bound to the ring A of formula (I);
R³ represents C₁₋₄-alkyl, or NR^{3a}R^{3b};
R^{3a} represents C₁₋₂-alkyl;
R^{3b} represents hydrogen, or methyl; and
   R⁴ represents methyl;
   and
   R⁵ represents C₁₋₄-alkyl, or NR^{5a}R^{5b};
R^{5a} represents C₁₋₃-alkyl;
R^{5b} represents hydrogen, or methyl; and
   R⁶ represents methyl.
   xii) Another embodiment of the invention relates to pyrimidine-pyridine derivatives according to any one of the embodiments i) to ix), wherein Pyridine¹ represents

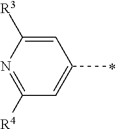

wherein the asterisk indicates the bond with which the pyridine ring is bound to the ring A of formula (I);
R³ represents C₁₋₄-alkyl, or Nee;
R^{3a} represents C₁₋₂-alkyl;
R^{3b} represents hydrogen, or methyl; and
   R⁴ represents methyl.
   xiii) A further embodiment of the invention relates to pyrimidine-pyridine derivatives according to any one of embodiments i), ii), and x) to xii), wherein
   A represents

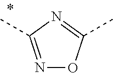

wherein the asterisk indicates the bond that is linked to the pyrimidine group of formula (I);
R¹ represents C₁₋₂-alkoxy, C₁₋₂-alkylamino, or N-methyl-N—C₁₋₂-alkyl-amino; and
R² represents methyl.
xiv) A further embodiment of the invention relates to pyrimidine-pyridine derivatives according to embodiment xiii), wherein
R¹ represents NHCH₃, N(CH₃)₂, or OCH₃.
xv) A further embodiment of the invention relates to pyrimidine-pyridine derivatives according to any one of embodiments i), ii), and x) to xii), wherein
A represents

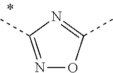

wherein the asterisk indicates the bond that is linked to the pyrimidine group of formula (I);
R¹ represents C₃₋₄-alkylamino, or N—C₃₋₄-alkyl-N—C₁₋₂-alkyl-amino; and
R² represents methyl.
xvi) Another embodiment of the invention relates to pyrimidine-pyridine derivatives according to any one of the embodiments i) to ix) and xiii) to xv), wherein
Pyridine¹ represents

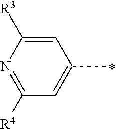

wherein the asterisk indicates the bond with which the pyridine ring is bound to the ring A of formula (I);
R³ represents C₃₋₄-alkyl; and
R⁴ represents C₁₋₂-alkyl.
xvii) Another embodiment of the invention relates to pyrimidine-pyridine derivatives according to any one of the embodiments i) to ix) and xiii) to xv), wherein
Pyridine¹ represents

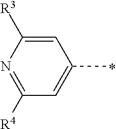

wherein the asterisk indicates the bond with which the pyridine ring is bound to the ring A of formula (I);
R³ represents CH₃; and
R⁴ represents C₁₋₂-alkyl.
xviii) A further embodiment of the invention relates to pyrimidine-pyridine derivatives according to embodiment i) or ii), wherein
A represents

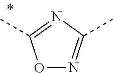

wherein the asterisk indicates the bond that is linked to the pyrimidine group of formula (I);
R¹ represents C₁₋₄-alkylamino, or N—C₁₋₄-alkyl-N—C₁₋₃-alkylamino;
R² represents C₁₋₂-alkyl; and
Pyridine¹ represents

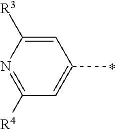

wherein the asterisk indicates the bond with which the pyridine ring is bound to the ring A of formula (I);
R³ represents methyl, ethyl, isopropyl, methylamino, or dimethylamino; and
R⁴ represents methyl, or chloro.
xix) A further embodiment of the invention relates to pyrimidine-pyridine derivatives according to embodiment i) or ii), wherein
A represents

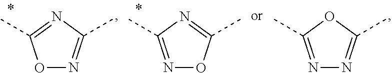

wherein the asterisks indicate the bond that is linked to the pyrimidine group of formula (I);
R¹ represents C₁₋₄-alkoxy, C₁₋₄-alkylamino, or N—C₁₋₄-alkyl-N—C₁₋₃-alkylamino;
R² represents C₁₋₂-alkyl; and
Pyridine¹ represents

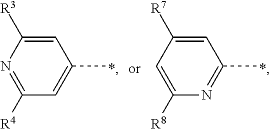

wherein the asterisks indicate the bond with which the pyridine ring is bound to the ring A of formula (I);
R³ represents C₁₋₄-alkyl, or Nee;
R^{3a} represents C₁₋₂-alkyl;
R^{3b} represents methyl; and
   R⁴ represents chloro, or C₁₋₂-alkyl;
   and
   R⁷ and R⁸ both represent C₁₋₂-alkyl.
   xx) A further embodiment of the invention relates to pyrimidine-pyridine derivatives according to embodiment i) or ii), wherein
   A represents

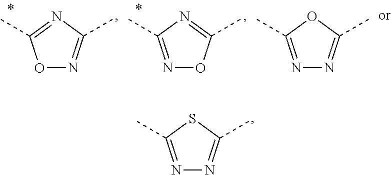

wherein the asterisks indicate the bond that is linked to the pyrimidine group of formula (I);
R¹ represents C₁₋₄-alkoxy, C₁₋₄-alkylamino, N—C₁₋₄-alkyl-N—C₁₋₃-alkylamino, cyclopropylamino, cyclopropylmethylamino, or pyrrolidine;
R² represents C₁₋₂-alkyl, or C₃₋₄-alkyl; and
Pyridine¹ represents

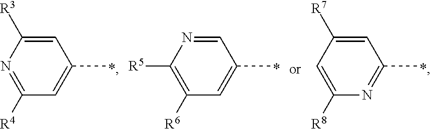

wherein the asterisks indicate the bond with which the pyridine ring is bound to the ring A of formula (I);
and in case R¹ represents C₁₋₄-alkylamino, N—C₁₋₄-alkyl-N—C₁₋₃-alkylamino, cyclopropylamino, or cyclopropylmethylamino, Pyridine¹ can also represent

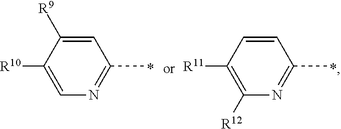

wherein the asterisks indicate the bond with which the pyridine ring is bound to the ring A of formula (I);
R³ represents C₁₋₄-alkyl, C₁₋₃-alkoxy such as methoxy, or NR^{3a}R^{3b};
R^{3a} represents C₁₋₂-alkyl;
R^{3b} represents hydrogen, or methyl; and
   R⁴ represents hydrogen, chloro, or C₁₋₂-alkyl such as methyl;
   R⁵ represents C₁₋₄-alkyl such as isobutyl, C₁₋₃-alkoxy such as isopropoxy, or NR^{5a}R^{5b};
R^{5a} represents C₁₋₃-alkyl such as isopropyl;
R^{5b} represents C₁₋₂-alkyl such as methyl; and
   R⁶ represents C₁₋₂-alkyl;
   R⁷ represents C₁₋₂-alkyl such as methyl, and R⁸ represents C₁₋₄-alkyl such as methyl or isobutyl;
   R⁹ represents C₁₋₂-alkyl such as methyl; and
   R¹⁰ represents C₁₋₄-alkyl such as methyl or ethyl;
   and
   R¹¹ represents C₁₋₄-alkyl; and
   R¹² represents C₁₋₂-alkyl such as methyl.

The compounds of formula (I) may contain one or more stereogenic or asymmetric centers, such as one or more asymmetric carbon atoms. The compounds of formula (I) may thus be present as mixtures of stereoisomers or preferably as pure stereoisomers. Mixtures of stereoisomers may be separated in a manner known to a person skilled in the art.

Where the plural form is used for compounds, salts, pharmaceutical compositions, diseases and the like, this is intended to mean also a single compound, salt, or the like.

Any reference hereinbefore or hereinafter to a compound of formula (I) is to be understood as referring also to salts, especially pharmaceutically acceptable salts, of a compound of formula (I), as appropriate and expedient.

The term “pharmaceutically acceptable salts” refers to non-toxic, inorganic or organic acid and/or base addition salts. Reference can be made to “Salt selection for basic drugs”, Int. J. Pharm. (1986), 33, 201-217.

Examples of preferred compounds are selected from the group consisting of:
{4-[3-(2,6-dimethyl-pyridin-4-yl)-[1,2,4]oxadiazol-5-yl]-6-methyl-pyrimidin-2-yl}-ethyl-amine;
ethyl-{4-[3-(2-ethyl-6-methyl-pyridin-4-yl)-[1,2,4]oxadiazol-5-yl]-6-methyl-pyrimidin-2-yl}-amine;
{4-[3-(2,6-dimethyl-pyridin-4-yl)-[1,2,4]oxadiazol-5-yl]-6-methyl-pyrimidin-2-yl}-propyl-amine;
{4-[3-(2-ethyl-6-methyl-pyridin-4-yl)-[1,2,4]oxadiazol-5-yl]-6-methyl-pyrimidin-2-yl}-propyl-amine;
{4-[3-(2,6-dimethyl-pyridin-4-yl)-[1,2,4]oxadiazol-5-yl]-6-methyl-pyrimidin-2-yl}-isopropyl-amine;
{4-[3-(2-ethyl-6-methyl-pyridin-4-yl)-[1,2,4]oxadiazol-5-yl]-6-methyl-pyrimidin-2-yl}-isopropyl-amine;
{4-[3-(2,6-dimethyl-pyridin-4-yl)-[1,2,4]oxadiazol-5-yl]-6-methyl-pyrimidin-2-yl}-isobutyl-amine;
{4-[3-(2,6-dimethyl-pyridin-4-yl)-[1,2,4]oxadiazol-5-yl]-6-methyl-pyrimidin-2-yl}-ethyl-methyl-amine;
{4-[3-(2,6-dimethyl-pyridin-4-yl)-[1,2,4]oxadiazol-5-yl]-6-methyl-pyrimidin-2-yl}-methyl-propyl-amine;
{4-[3-(2,6-dimethyl-pyridin-4-yl)-[1,2,4]oxadiazol-5-yl]-6-methyl-pyrimidin-2-yl}-isobutyl-methyl-amine;
{4-[3-(2,6-dimethyl-pyridin-4-yl)-[1,2,4]oxadiazol-5-yl]-6-ethyl-pyrimidin-2-yl}-ethyl-amine;
{4-[5-(2-Isobutyl-6-methyl-pyridin-4-yl)-[1,2,4]oxadiazol-3-yl]-6-methyl-pyrimidin-2-yl}-dimethyl-amine; and
{4-[5-(2-isobutyl-6-methyl-pyridin-4-yl)-[1,2,4]oxadiazol-3-yl]-6-methoxy-6-methyl-pyrimidine.

Examples of preferred compounds are further selected from the group consisting of:
isopropyl-{4-methyl-6-[3-(2-methyl-pyridin-4-yl)-[1,2,4]oxadiazol-5-yl]-pyrimidin-2-yl}-amine;
{4-[3-(2,6-dimethyl-pyridin-4-yl)-[1,2,4]oxadiazol-5-yl]-6-methyl-pyrimidin-2-yl}-isopropyl-methyl-amine;
{4-[3-(2,6-dimethyl-pyridin-4-yl)-[1,2,4]oxadiazol-5-yl]-6-methyl-pyrimidin-2-yl}-diethyl-amine;
{4-[3-(2,6-dimethyl-pyridin-4-yl)-[1,2,4]oxadiazol-5-yl]-6-methyl-pyrimidin-2-yl}-ethyl-propyl-amine;
{4-[3-(2,6-dimethyl-pyridin-4-yl)-[1,2,4]oxadiazol-5-yl]-6-methyl-pyrimidin-2-yl}-ethyl-isopropyl-amine;
cyclopropyl-{4-[3-(2,6-dimethyl-pyridin-4-yl)-[1,2,4]oxadiazol-5-yl]-6-methyl-pyrimidin-2-yl}-amine;
cyclopropyl-{4-[3-(2-ethyl-6-methyl-pyridin-4-yl)-[1,2,4]oxadiazol-5-yl]-6-methyl-pyrimidin-2-yl}-amine;
isopropyl-{4-[3-(2-methoxy-6-methyl-pyridin-4-yl)-[1,2,4]oxadiazol-5-yl]-6-methyl-pyrimidin-2-yl}-amine;
4-[3-(2,6-dimethyl-pyridin-4-yl)-[1,2,4]oxadiazol-5-yl]-2-isopropoxy-6-methyl-pyrimidine;
4-[3-(2,6-dimethyl-pyridin-4-yl)-[1,2,4]oxadiazol-5-yl]-2-isobutoxy-6-methyl-pyrimidine;
{4-[3-(2,6-dimethyl-pyridin-4-yl)-[1,2,4]oxadiazol-5-yl]-6-propyl-pyrimidin-2-yl}-methyl-amine;
{4-[3-(2,6-dimethyl-pyridin-4-yl)-[1,2,4]oxadiazol-5-yl]-6-propyl-pyrimidin-2-yl}-dimethyl-amine;
{4-[3-(2,6-dimethyl-pyridin-4-yl)-[1,2,4]oxadiazol-5-yl]-6-ethyl-pyrimidin-2-yl}-propyl-amine;
{4-[3-(2,6-dimethyl-pyridin-4-yl)-[1,2,4]oxadiazol-5-yl]-6-ethyl-pyrimidin-2-yl}-isopropyl-amine;
{4-[3-(2,6-dimethyl-pyridin-4-yl)-[1,2,4]oxadiazol-5-yl]-6-ethyl-pyrimidin-2-yl}-isobutyl-amine;
{4-[3-(2,6-dimethyl-pyridin-4-yl)-[1,2,4]oxadiazol-5-yl]-6-ethyl-pyrimidin-2-yl}-isobutyl-methyl-amine;
{4-[5-(2,6-dimethyl-pyridin-4-yl)-[1,2,4]oxadiazol-3-yl]-6-methyl-pyrimidin-2-yl}-propyl-amine;
{4-[5-(2,6-dimethyl-pyridin-4-yl)-[1,2,4]oxadiazol-3-yl]-6-methyl-pyrimidin-2-yl}-isopropyl-amine;
{4-[5-(2,6-dimethyl-pyridin-4-yl)-[1,2,4]oxadiazol-3-yl]-6-methyl-pyrimidin-2-yl}-isobutyl-methyl-amine;
{4-[5-(2,6-dimethyl-pyridin-4-yl)-[1,2,4]oxadiazol-3-yl]-6-methyl-pyrimidin-2-yl}-diethyl-amine;
{4-[5-(2,6-dimethyl-pyridin-4-yl)-[1,2,4]oxadiazol-3-yl]-6-methyl-pyrimidin-2-yl}-ethyl-propyl-amine; and
{4-[5-(2,6-dimethyl-pyridin-4-yl)-[1,2,4]oxadiazol-3-yl]-6-methyl-pyrimidin-2-yl}-ethyl-isopropyl-amine.

The compounds of formula (I) and their pharmaceutically acceptable salts can be used as medicaments, e.g. in the form of pharmaceutical compositions for enteral or parenteral administration, and are suitable for decreasing the number of circulating lymphocytes and for the prevention and/or treatment of diseases or disorders associated with an activated immune system.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of formula (I) or their pharmaceutically acceptable salts, optionally in combination with other therapeutically valuable substances, into a galenical administration form together with suitable, non-toxic, inert, pharmaceutically acceptable solid or liquid carrier materials and, if desired, usual pharmaceutical adjuvants.

The pharmaceutical compositions comprising a compound of formula (I) are useful for the prevention and/or treatment of diseases or disorders associated with an activated immune system.

Such diseases or disorders associated with an activated immune system and to be prevented/treated with the compounds of formula (I) are for example selected from the group consisting of rejection of transplanted organs, tissue or cells; graft-versus-host diseases brought about by transplantation; autoimmune syndromes including rheumatoid arthritis; systemic lupus erythematosus; antiphospholipid syndrome; Hashimoto's thyroiditis; lymphocytic thyroiditis; multiple sclerosis; myasthenia gravis; type I diabetes; uveitis; episcleritis; scleritis; Kawasaki's disease, uveo-retinitis; posterior uveitis; uveitis associated with Behcet's disease; uveomeningitis syndrome; allergic encephalomyelitis; chronic allograft vasculopathy; post-infectious autoimmune diseases including rheumatic fever and post-infectious glomerulonephritis; inflammatory and hyperproliferative skin diseases; psoriasis; psoriatic arthritis; atopic dermatitis; myopathy; myositis; osteomyelitis; contact dermatitis; eczematous dermatitis; seborrhoeic dermatitis; lichen planus; pemphigus; bullous pemphigoid; epidermolysis bullosa; urticaria; angioedema; vasculitis; erythema; cutaneous eosinophilia; acne; scleroderma; alopecia greata; keratoconjunctivitis; vernal conjunctivitis; keratitis; herpetic keratitis; dystrophia epithelialis corneae; corneal leukoma; ocular pemphigus; Mooren's ulcer; ulcerative keratitis; scleritis; Graves' ophthalmopathy; Vogt-Koyanagi-Harada syndrome; sarcoidosis; pollen allergies; reversible obstructive airway disease; bronchial asthma; allergic asthma; intrinsic asthma; extrinsic asthma; dust asthma; chronic or inveterate asthma; late asthma and airway hyper-responsiveness; bronchiolitis; bronchitis; endometriosis; orchitis; gastric ulcers; ischemic bowel diseases; inflammatory bowel diseases; necrotizing enterocolitis; intestinal lesions associated with thermal burns; coeliac disease; proctitis; eosinophilic gastroenteritis; mastocytosis; Crohn's disease; ulcerative colitis; vascular damage caused by ischemic diseases and thrombosis; atherosclerosis; fatty heart; myocarditis; cardiac infarction; aortitis syndrome; cachexia due to viral disease; vascular thrombosis; migraine; rhinitis; eczema; interstitial nephritis; IgA-induced nephropathy; Goodpasture's syndrome; hemolytic-uremic syndrome; diabetic nephropathy; glomerulosclerosis; glomerulonephritis; tubulointerstitial nephritis; interstitial cystitis; multiple myositis; Guillain-Barré syndrome; Meniere's disease; polyneuritis; multiple neuritis; myelitis; mononeuritis; radiculopathy; hyperthyroidism; Basedow's disease; thyrotoxicosis; pure red cell aplasia; aplastic anemia; hypoplastic anemia; idiopathic thrombocytopenic purpura; autoimmune hemolytic anemia; autoimmune thrombocytopenia; agranulocytosis; pernicious anemia; megaloblastic anemia; anerythroplasia; osteoporosis; fibroid lung; idiopathic interstitial pneumonia; dermatomyositis; leukoderma vulgaris; ichthyosis vulgaris; photoallergic sensitivity; cutaneous T cell lymphoma; polyarteritis nodosa; Huntington's chorea; Sydenham's chorea; myocardosis; myocarditis; scleroderma; Wegener's granuloma; Sjogren's syndrome; adiposis; eosinophilic fascitis; lesions of gingiva, periodontium, alveolar bone, substantia ossea dentis; male pattern alopecia or alopecia senilis; muscular dystrophy; pyoderma; Sezary's syndrome; hypophysitis; chronic adrenal insufficiency; Addison's disease; ischemia-reperfusion injury of organs which occurs upon preservation; endotoxin shock; pseudomembranous colitis; colitis caused by drug or radiation; ischemic acute renal insufficiency; chronic renal insufficiency; lung cancer; malignancy of lymphoid origin; acute or chronic lymphocytic leukemias; lymphoma; pulmonary emphysema; cataracta; siderosis; retinitis pigmentosa; senile macular degeneration; vitreal scarring; corneal alkali burn; dermatitis erythema; ballous dermatitis; cement dermatitis; gingivitis; periodontitis; sepsis; pancreatitis; peripheral artery disease; carcinogenesis; solid cancer tumors; metastasis of carcinoma; hypobaropathy; autoimmune hepatitis; primary biliary cirrhosis; sclerosing cholangitis; partial liver resection; acute liver necrosis; cirrhosis; alcoholic cirrhosis; hepatic failure; fulminant hepatic failure; late-onset hepatic failure; and “acute-on-chronic” liver failure.

Preferred diseases or disorders to be treated and/or prevented with the compounds of formula (I) are selected from the group consisting of rejection of transplanted organs such as kidney, liver, heart, lung, pancreas, cornea, and skin; graft-versus-host diseases brought about by stem cell transplantation; autoimmune syndromes including rheumatoid arthritis, multiple sclerosis, inflammatory bowel diseases such as Crohn's disease and ulcerative colitis, psoriasis, psoriatic arthritis, thyroiditis such as Hashimoto's thyroiditis, uveo-retinitis; atopic diseases such as rhinitis, conjunctivitis, dermatitis; asthma; type I diabetes; post-infectious autoimmune diseases including rheumatic fever and post-infectious glomerulonephritis; solid cancers and tumor metastasis.

Particularly preferred diseases or disorders to be treated and/or prevented with the compounds of formula (I) are selected from the group consisting of rejection of transplanted organs selected from kidney, liver, heart and lung; graft-versus-host diseases brought about by stem cell transplantation; autoimmune syndromes selected from rheumatoid arthritis, multiple sclerosis, psoriasis, psoriatic arthritis, Crohn's disease, and Hashimoto's thyroiditis; and atopic dermatitis. Very preferably the diseases or disorders to be treated and/or prevented with the compounds of formula (I) are selected from multiple sclerosis and psoriasis.

The present invention also relates to a method for the prevention or treatment of a disease or disorder mentioned herein comprising administering to a subject a pharmaceutically active amount of a compound of formula (I).

Furthermore, compounds of the formula (I) are also useful, in combination with one or several immunomodulating agents, for the prevention and/or treatment of the diseases and disorders mentioned herein. According to a preferred embodiment of the invention, said agents are selected from the group consisting of immunosuppressants, corticosteroids, NSAID's, cytotoxic drugs, adhesion molecule inhibitors, cytokines, cytokine inhibitors, cytokine receptor antagonists and recombinant cytokine receptors.

The present invention also relates to the use of a compound of formula (I) for the preparation of a pharmaceutical composition, optionally for use in combination with one or several immunomodulating agents, for the prevention or treatment of the diseases and disorders mentioned herein.

The compounds of formula (I) can be manufactured by the methods given below, by the methods given in the Examples or by analogous methods. Optimum reaction conditions may vary with the particular reactants or solvents used, but such conditions can be determined by a person skilled in the art by routine optimisation procedures.

Compounds of the formula (I) of the present invention can be prepared according to the general sequence of reactions outlined below. Only a few of the synthetic possibilities leading to compounds of formula (I) are described.

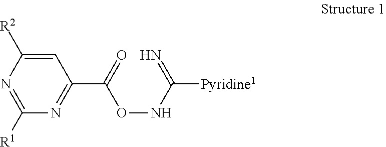

Compounds of formula (I) which represent a 5-pyrimidin-4-yl-[1,2,4]oxadiazole derivative, are prepared by reacting a compound of Structure 1 in a solvent such as dioxane, THF, dimethoxyethane, xylene, toluene, benzene, pyridine, DMF, dichloromethane, acetic acid, trifluoroacetic acid, etc. at rt or elevated temperatures in the presence or absence of auxiliaries such as acids (e.g. TFA, acetic acid, HCl, etc.), bases (e.g. NaH, NaOAc, Na₂CO₃, K₂CO₃, triethylamine, etc.), tetraalkylammonium salts, or water removing agents (e.g. oxalyl chloride, a carboxylic acid anhydride, POCl₃, PCl_{S}, P₄O₁₀, molecular sieves, methoxycarbonylsulfamoyl triethylammonium hydroxide (Burgess reagent), etc.) (Lit.: e.g. A. R. Gangloff, J. Litvak, E. J. Shelton, D. Sperandio, V. R. Wang, K. D. Rice, Tetrahedron Lett. 42 (2001), 1441-1443; T. Suzuki, K. lwaoka, N. Imanishi, Y. Nagakura, K. Miyta, H. Nakahara, M. Ohta, T. Mase, Chem. Pharm. Bull. 47 (1999), 120-122; R. F. Poulain, A. L. Tartar, B. P. Déprez, Tetrahedron Lett. 42 (2001), 1495-1498; R. M. Srivastava, F. J. S. Oliveira, D. S. Machado, R. M. Souto-Maior, Synthetic Commun. 29 (1999), 1437-1450; E. O. John, J. M. Shreeve, Inorganic Chemistry 27 (1988), 3100-3104; B. Kaboudin, K. Navaee, Heterocycles 60 (2003), 2287-2292; C. T. Brain, J. M. Paul, Y. Loong, P. J. Oakley, Tetrahedron Lett. 40 (1999), 3275-3278).

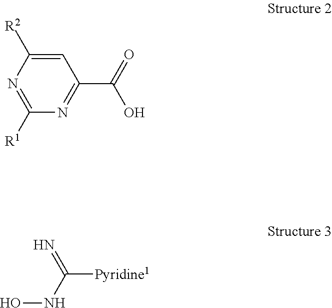

Compounds of Structure 1 may be prepared by reacting a compound of Structure 2 with a compound of Structure 3 in a solvent such as DMF, THF, DCM, etc. in the presence or absence of one or more coupling agents such as TBTU, DCC, EDC, HBTU, HOBt, CDI, PyBOP, etc. and in the presence or absence of a base such as triethylamine, DIPEA, NaH, K₂CO₃, etc. (Lit.: e.g. A. Hamze, J.-F. Hernandez, P. Fulcrand, J. Martinez, J. Org. Chem. 68 (2003), 7316-7321; and the literature cited above).

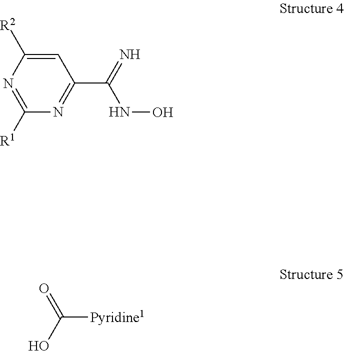

Compounds of formula (I) which represent a 3-pyrimidin-4-yl-[1,2,4]oxadiazole derivative are prepared in an analogous fashion (Lit.: e.g. C. T. Brain, J. M. Paul, Y. Loong, P. J. Oakley, Tetrahedron Lett. 40 (1999), 3275-3278; W. J. Fanshawe, S. R. Safir U.S. Pat. No. 3,857,843 (American Cyanamid Co., USA)) by reacting a compound of Structure 4 with a compound of Structure 5 and subsequent cyclisation of the corresponding hydroxyamidine ester intermediate.

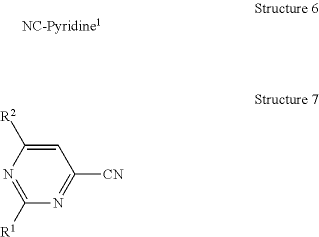

Compounds of Structure 3 and 4 may be prepared by reacting a compound of Structure 6 and 7, respectively, with hydroxylamine or one of its salts in a solvent such as methanol, ethanol, pyridine, etc. in the presence or absence of a base such as Na₂CO₃, K₂CO₃, triethylamine, KOtBu, etc. (Lit.: e.g. T. Suzuki, K. lwaoka, N. Imanishi, Y. Nagakura, K. Miyta, H. Nakahara, M. Ohta, T. Mase, Chem. Pharm. Bull. 47 (1999), 120-122; J. Cui, D. Crich, D. Wink, M. Lam, A. L. Rheingold, D. A. Case, W. T. Fu, Y. Zhou, M. Rao, A. J. Olson, M. E. Johnson, Bioorg. Med. Chem. 11 (2003), 3379-3392; R. Miller, F. Lang, Z. J. Song, D. Zewge, WO 2004/035538 (Merck & Co., Inc., USA); B. Kaboudin, K. Navaee, Heterocycles 60 (2003), 2287-2292).

Compounds of Structure 6 are either commercially available or are prepared from compounds of Structure 5 according to procedures described herein or according to procedures known to a person skilled in the art.

Methods that effect the transformation of a compound of Structure 2 into a compound of Structure 7, or the opposite, are known to a person skilled in the art and described herein.

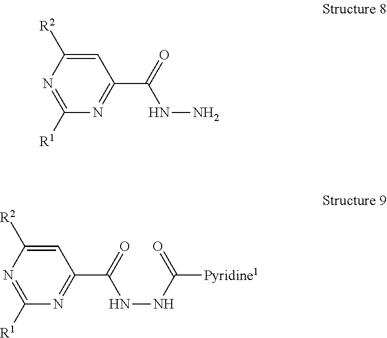

Compounds of formula (I) which represent a 2-pyrimidin-4-yl-[1,3,4]oxadiazole or a 2-pyrimidin-4-yl-[1,3,4]thiadiazole derivative are prepared similarly by reacting a compound of Structure 2 with hydrazine (by using a coupling reagent such as TBTU, DCC, EDC, HBTU, PyBOP, HOBt, CDI, etc.) to form a compound of Structure 8 which is then coupled with a compound of Structure 5 to give a compound of Structure 9. A compound of Structure 9 can also be prepared by following the reverse reaction order i.e. by first coupling a compound of Structure 5 with hydrazine followed by reacting the corresponding hydrazide intermediate with a compound of Structure 2. Dehydration of a compound of Structure 9 to form the desired 2-pyrimidin-4-yl-[1,3,4]oxadiazole derivative is affected by treating a compound of Structure 9 with a reagent such as POCl₃, CCl₄ or CBr₄ in combination with triphenylphosphine, P₂O₅, Burgess reagent, etc. in a solvent such as toluene, acetonitrile, dioxane, THF, CHCl₃, etc. at temperatures between 20° C. and 120° C. in the presence or absence of microwave irradiation. (Lit.: e.g. M. A. Garcia, S. Martin-Santamaria, M. Cacho, F. Moreno de la Llave, M. Julian, A. Martinez, B. De Pascual-Teresa, A. Ramos, J. Med. Chem. 48 (2005), 4068-4075; C. T. Brain, J. M. Paul, Y. Loong, P. J. Oakley, Tetrahedron Lett. 40 (1999), 3275-3278). Likewise, 2-pyrimidin-4-yl-[1,3,4]thiadiazole derivatives are obtained by cyclising a compound of Structure 9 with Lawesson's reagent, optionally in combination with P₂S₅, in the presence or absence of a solvent such as pyridine, toluene, THF, acetonitrile, etc. at elevated temperatures with or without microwave irradiation (Lit.: e.g. A. A. Kiryanov, P. Sampson, A. J. Seed, J. Org. Chem. 66 (2001), 7925-7929; Org. Prep. Proc. Int. 37 (2005), 213-222).

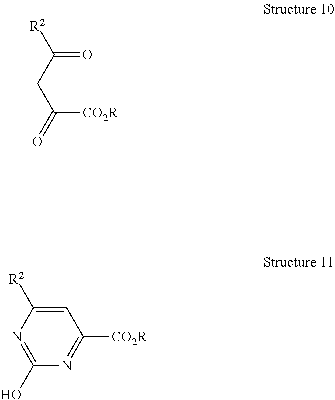

Compounds of Structure 2 may be prepared by reacting a 2,4-dioxo-alkanoic ester (Structure 10, wherein R represents a C₁₋₄-alkyl group (especially an ethyl group) and R² represents preferably a methyl or an ethyl) with urea in acidic medium in the presence or absence of an additional solvent such as methanol, ethanol, dioxane, etc., preferably at temperatures above 50° C. to give a compound of Structure 11. The compounds of Structure 11 can then be reacted with POCl₃ (Lit.: e.g. Palanki, M. S. S., Erdman, P. E., Gayo-Fung, L. M., Shelvin, G. I., Sullivan, R. W., Suto, M. J., Goldman, M. E., Ransone, L. J., Bennett, B. L., Manning, A. M., J. Med. Chem. 43 (2000), 3995-4004; Z. Budesinsky, F. Roubinek, Collection Czechoslov. Chem. Commun. 26 (1961), 2871-2885) to give a compound of Structure 12, which can be hydrolysed to a compound of Structure 13. The compounds of Structure 13 can be reacted with the appropriate amine or alcohol in combination with a base such as Hünig's base or NaOH, in the presence or absence of an additional solvent such as THF, dioxane etc., preferably at temperatures above 50° C. to give a compound of Structure 2.

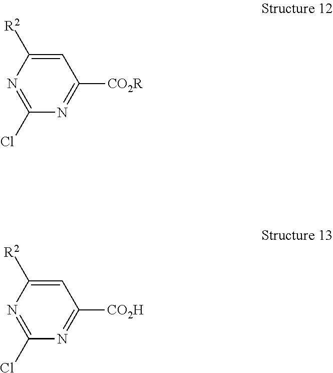

In case R¹ represents a monoalkylamino group, the corresponding monoalkylamino-pyrimidine derivatives that may occur in the course of the synthesis of compounds of formula (I), may require temporary protection at the secondary amine function.

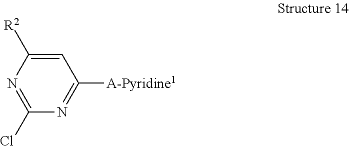

The above described reaction sequences that allow the introduction of the two residues R¹ and R² may also be applied to a compound in which the scaffold has already been further elaborated. For instance, the desired residue R¹ may also be introduced in a later step from a compound of Structure 14 which can be synthesised by methods analogous to those described herein, e.g. by the coupling-cyclisation sequence of the pyrimidine compounds of Structure 13 with the pyridine compounds of Structure 3.

Alternatively, the compounds of Structure 2 may also be prepared by reacting a compound of Structure 10 with S-methylisothiourea sulphate in the presence or absence of an additional solvent such as methanol, ethanol, dioxane, etc., preferably at temperatures above 50° C. to give a compound of Structure 15. The compounds of Structure 15 can then be hydrolysed under basic conditions to the corresponding carboxylic acid derivatives which are reacted with oxidation agent such as mCPBA to give the compounds of Structure 16 (Lit.: e.g. Z. Budesinsky, F. Roubinek, Collection Czechoslov. Chem. Commun. 26 (1961), 2871-2885). The compounds of Structure 16 can be reacted with the appropriate amine or alcohol in combination with a base such as Hünig's base or NaOH, in the presence or absence of an additional solvent such as THF, dioxane etc., preferably at temperatures above 50° C. to give a compound of Structure 2.

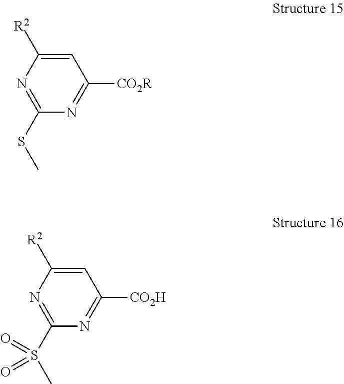

Compounds of Structure 5, wherein Pyridine¹ represents

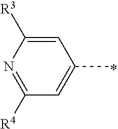

may be prepared by reacting a 2,6-dichloro-isonicotinic acid ester (Structure 17, below) with an alkyl Grignard reagent in the presence of Fe(acac)₃ in a solvent such as THF, dioxane, DMF, NMP, etc., or combinations thereof, at temperatures ranging from −78 to 25° C. (Fürstner conditions, Lit.: e.g. A. Fürstner, A. Leitner, M. Mendez, H. Krause J. Am. Chem. Soc. 124 (2002) 13856-13863; A. Fürstner, A. Leitner, Angew. Chem. 114 (2002) 632-635). The reaction conditions can be chosen such that either the 2-chloro-6-alkyl-isonicotinic acid ester or the 2,6-dialkyl-isonicotinic acid ester is obtained as the main product. The two chlorine atoms in a 2,6-dichloro-isonicotinic acid ester may also be substituted either sequentially or in one step by two alk-1-enyl groups, which may be the same or different, by treating 2,6-dichloro-isonicotinic acid ester with the appropriate alkenyl boron derivative under Suzuki coupling conditions known to a person skilled in the art. The obtained 2,6-di-alkenyl-isonicotinic acid ester is hydrogenated to the corresponding 2,6-dialkyl-isonicotinic acid ester. In addition, a procedure in which the Fürstner and the Suzuki conditions are employed sequentially can be envisaged. The 2,6-dichloro-isonicotinic acid esters or the 2-chloro-6-alkyl-isonicotinic acid esters may also be treated with an alcohol or an alcoholate at elevated temperatures to furnish the corresponding 2-chloro-6-alkoxy-isonicotinic acid esters or 2-alkoxy-6-alkyl-isonicotinic acid esters (Lit.: e.g. N. Wild, U. Groth, Eur. J. Org. Chem. 2003, 4445-4449). Finally, cleavage of the ester functionality delivers compounds of Structure 5.

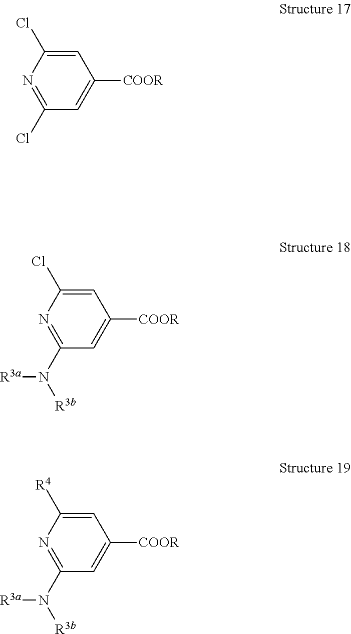

Compounds of the above Structure 5 wherein R³ represents NR^{3a}R^{3b} may be prepared by reacting a 2,6-dichloro-isonicotinic acid ester (Structure 17, wherein R represents a C₁₋₄-alkyl, preferably an isopropyl or a tert.-butyl group) with the appropriate amine NHR^{3a}R^{3b} in the presence or absence of an additional solvent such as THF, dioxane, ethanol, etc., preferably at temperatures above 50° C. to give a compound of Structure 18. The compounds of Structure 18 can then be reacted with the appropriate alkyl-Zn reagent (e.g. Me₂Zn, MeZnCl, Et₂Zn, etc.) under Negishi reaction conditions (Lit.: e.g. H. Matsushita, E. Negishi, J. Org. Chem. 47 (1982) 4161-4165) to give a compound of Structure 19, which can be hydrolysed to a compound of Structure 5. In addition, compounds of the Structure 19 may be prepared by reacting a compound of Structure 18 with an alkyl Grignard reagent in the presence of Fe(acac)₃ in a solvent such as THF, dioxane, DMF, NMP, etc., or combinations thereof, at temperatures ranging from −78 to 25° C. (Fürstner conditions, see above). In case R⁴ represents an ethyl group, the corresponding compounds of Structure 19 can also be prepared by reacting a compound of Structure 18 with an alkenyl boron derivative (e.g. 2,4,6-trivinyl-cyclotriboroxane) under Suzuki conditions (Lit.: e.g. F. Kerins, D. F. O'Shea, J. Org. Chem. 67 (2002) 4968-4971). The obtained 2-amino-6-alkenyl-isonicotinic acid derivative is hydrogenated to the corresponding compound of Structure 19.

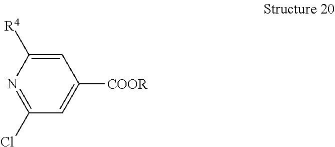

Alternatively, the compounds of Structure 19 may also be prepared by reacting a compound of Structure 20 with the appropriate amine NHR^{3a}R^{3b} under Buchwald-Hartwig conditions (Lit.: e.g. J. P. Wolfe, H. Tomori, J. P. Sadighi, J. Yin, S. L. Buchwald, J. Org. Chem. 65 (2000) 1158-1174; S. Wagaw, S. L. Buchwald, J. Org. Chem. 61 (1996) 7240-7241; M. C. Harris, O. Geis, S. L. Buchwald, J. Org. Chem. 64 (1999) 6019-6022; S. R. Stauffer, S. Lee, J. P. Stambuli, S. I. Hauck, J. F. Hartwig, Org. Letters 2 (2000) 1423-1426). Compounds of Structure 20 or their corresponding acids are either commercially available or may be prepared by reacting a 2,6-dichloro-isonicotinic acid ester (Structure 17) with an alkyl Grignard reagent under Fürstner conditions (see above) or with an alkyl-Zn reagent under Negishi conditions. Reacting a compound of Structure 17 with an alkenyl boron derivative under Suzuki conditions, treating the corresponding alkenyl-chloro-isonicotinic acid ester with an amine NHR^{3a}R^{3b} under Buchwald-Hartwig conditions and subsequent hydrogenation may also give access to compounds of Structure 19. The residues R^{3a} and R^{3b} may also be introduced by sequencial alkylation and/or reductive amination of a compound of Structure 21 (Lit.: e.g. N. Finch, T. R. Campbell, C. W. Gemenden, H. J. Povalski, J. Med. Chem. 23 (1980) 1405-1410) which may be prepared by reacting a compound of Structure 20 with ammonia in a solvent such as water, methanol, ethanol, THF, etc. at elevated temperatures.

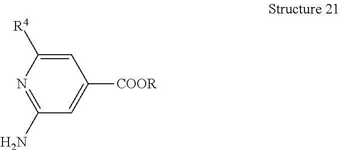

In case R^{3b} represents hydrogen, the corresponding pyridine derivatives that may occur in the course of the synthesis of compounds of formula (I), may require temporary protection at the secondary amine function.

Compounds of the Structure 5 wherein Pyridine¹ represents

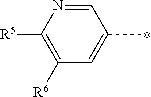

may be prepared by reacting a 5,6-dichloronicotinic acid ester with an alkyl Grignard reagent in the presence of Fe(acac)₃ in a solvent such as THF, dioxane, DMF, NMP, etc., or combinations thereof, at temperatures ranging from −78° C. to 25° C. (Fürstner conditions, Lit.: e.g. A. Fürstner, A. Leitner, M. Mendez, H. Krause, J. Am. Chem. Soc. 124 (2002) 13856-13863; A. Fürstner, A. Leitner, Angew. Chem. 114 (2002) 632-635). The reaction conditions can be chosen such that either the 5-chloro-6-alkyl-nicotinic acid ester or the 5,6-dialkyl-nicotinic acid ester is obtained as the main product. The two chlorine atoms in a 5,6-dichloronicotinic acid ester may also be substituted either sequentially or in one step by two alk-1-enyl groups, which may be the same or different, by treating 5,6-dichloronicotinic acid ester with the appropriate alkenyl boron derivative under Suzuki coupling conditions known to a person skilled in the art. The obtained 5,6-di-alkenyl-nicotinic acid ester is hydrogenated to the corresponding 5,6-dialkyl-nicotinic acid ester. In addition, a procedure in which the Fürstner and the Suzuki conditions are employed sequentially can be envisaged. Furthermore, chloronicotinic acids may also be transformed to the corresponding alkylnicotinic acid using the Negishi reaction (see above). The 5,6-dichloronicotinic acid ester may also be treated with an alcohol or an alcoholate at elevated temperatures to furnish the corresponding 5-chloro-6-alkoxy-nicotinic acid esters. Finally, cleavage of the ester functionality delivers compounds of Structure 5.

Alternatively, compounds of Structure 5, wherein R⁶ represents a methyl group, can be prepared from a compound of Structure 22 via formation of the corresponding 6-chloro-5-methyl-nicotinic acid esters using methods well known in the art, followed by derivatisation using Fürstner or Suzuki conditions as described above and subsequent cleavage of the ester function. The compound of Structure 22 can be prepared from known 6-chloro-3-formyl-5-methyl-pyridine (Lit.: e.g. EP-0702003) by oxidation of the formyl group to the carboxylic acid using oxidation reagents well known in the art such as aq. H₂O₂ in formic acid, KMnO₄, etc. in the presence or absence of a solvent such as toluene, THF, acetonitrile, acetone, etc. at temperatures between 0° C. and 120° C. The corresponding nitrile of Structure 6, wherein R⁶ represents a methyl group, can be prepared according to literature methods (Lit.: e.g. J. B. Paine III, J. Heterocyclic Chem. 1987, 351-355).

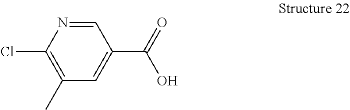

Compounds of Structure 5 wherein Pyridine¹ represents

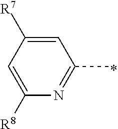

(Structure 23) are commercially available when R⁷═R⁸═CH₃ or may be prepared following the reaction sequence outlined below:

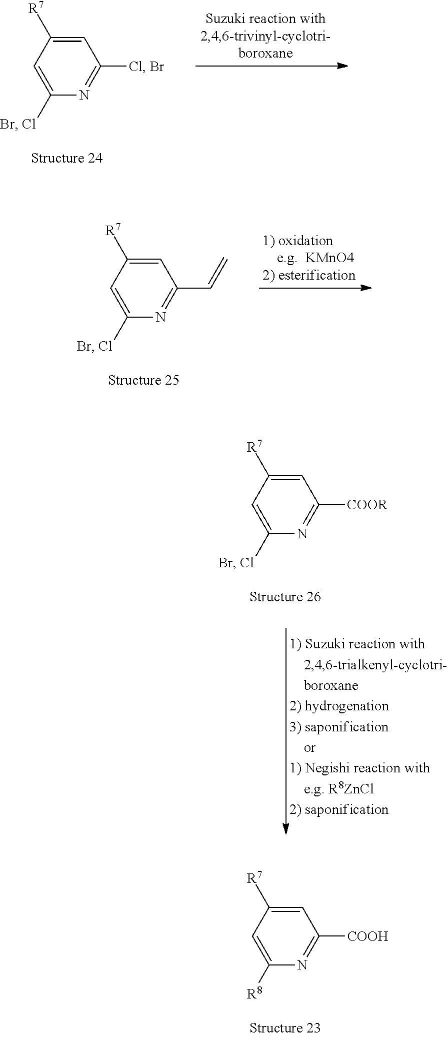

The picolinic acid of Structure 23 may be prepared by treating a compound of Structure 24 (either commercially available or prepared in analogy to literature procedures, e.g. T. Kaminski, P. Gros, Y. Fort, Eur. J. Org. Chem. 19 (2003) 3855-3860; U. Ziener, E. Breuning, J.-M. Lehn, E. Wegelius, K. Rissanen, G. Baum, D. Fenske, G. Vaughan, Chemistry—A European Journal 6 (2000) 4132-4139; R.-A. Fallahpour, Synthesis 2000 1665-1667) with 2,4,6-trivinyl-cyclotriboroxane under Suzuki conditions to form a compound of Structure 25 which is oxidised and esterified to the picolinic acid of Structure 26. A compound of Structure 26 (wherein R represents a C₁₋₂-alkyl) is then either subjected to Suzuki cross coupling conditions using the appropriate 2,4,6-trialkenyl-cyclotriboroxane (prepared according to F. Kerins, D. F. O'Shea, J. Org. Chem. 67 (2002) 4968-4971), hydrogenated and saponified, or treated with the appropriate alkyl-Zn-reagent under Negishi conditions prior to saponification to furnish the desired compound of Structure 23.

Compounds of the Structure 5 wherein Pyridine¹ represents

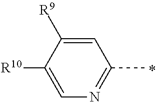

(Structure 27 or Structure 33) may be prepared following the reaction sequence outlined below:

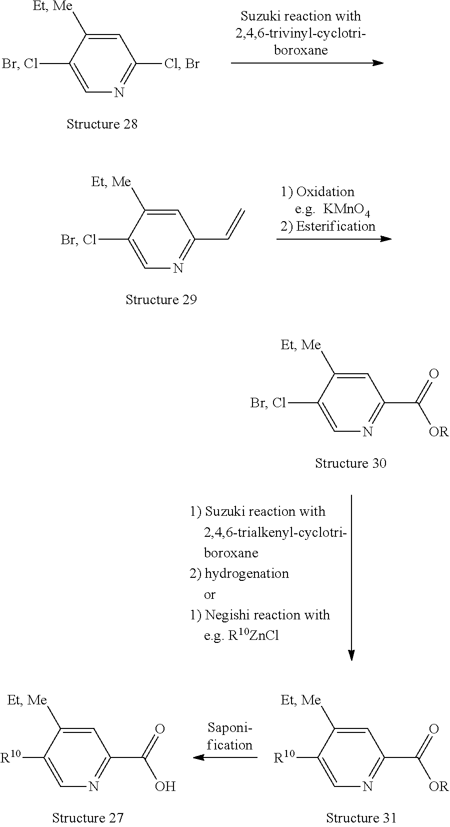

Thus, a compound of Structure 28 (commercially available or may be prepared in analogy to literature procedures, e.g. P. Pierrat, P. Gros, Y. Fort, Synlett 2004, 2319-2322) is reacted with 2,4,6-trivinyl-cyclotriboroxane under Suzuki conditions to form a compound of Structure 29, which is oxidised and esterified to a compound of Structure 30 (wherein R represents a C₁₋₂-alkyl). Suzuki reaction with the appriopriate 2,4,6-trialkenyl-cyclotriboroxane, hydrogenation and saponification or Negishi reaction with the appropriate alkyl-Zn-reagent followed by saponification of a compound of Structure 31 furnish the compounds of Structure 27.

Compounds of Structure 5 wherein Pyridine¹ represents

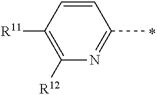

(Structure 32) may be prepared following the reaction sequence outlined below:

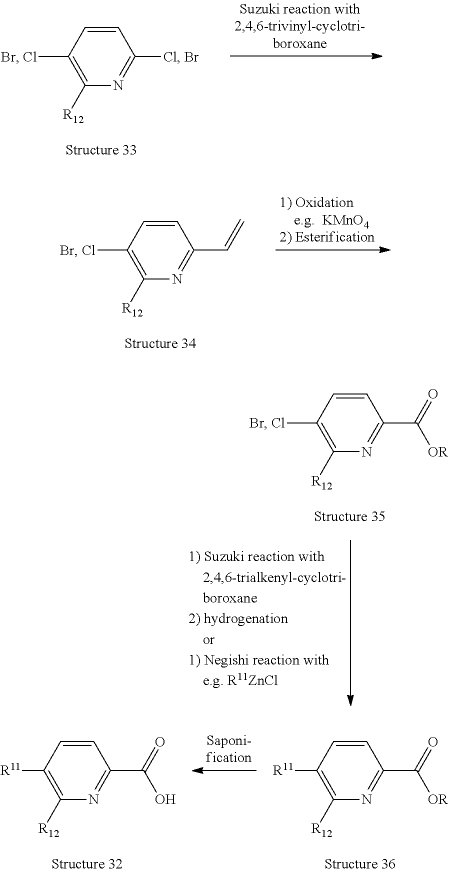

Thus, a compound of Structure 33 is treated with 2,4,6-trivinyl-cyclotriboroxane under Suzuki conditions to give a compound of Structure 34. Oxidation followed by esterification gives the corresponding compound of Structure 35 (wherein R represents a C₁₋₂-alkyl). Suzuki reaction with the appropriate 2,4,6-trialkenyl-cyclotriboroxane, hydrogenation and saponification or Negishi reaction with the appropriate alkyl-Zn-reagent followed by saponification furnishes the desired compounds of Structure 32. Compounds of Structure 33, wherein R¹² represents a methyl group are commercially available. Compounds of Structure 33, wherein R¹² represents an ethyl group can be prepared following literature procedures (e.g. WO 2006/097817 (Pfizer Japan Inc.), p 84; S. R. Natarajan et al. Bioorg. Med. Chem. Lett. 13 (2003) 273-276), for instance from commercially available 3-amino-2,6-dichloropyridine as outlined below:

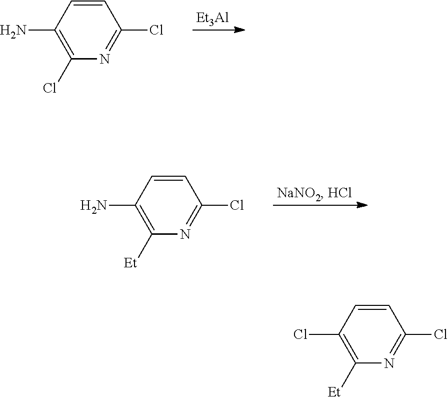

Whenever the compounds of formula (I) are obtained in the form of mixtures of enantiomers, the enantiomers can be separated using methods known to one skilled in the art: e.g. by formation and separation of diastereomeric salts or by HPLC over a chiral stationary phase such as a Regis Whelk-O1(R,R) (10 μm) column, a Daicel ChiralCel OD-H (5-10 μm) column, or a Daicel ChiralPak IA (10 μm) or AD-H (5 μm) column. Typical conditions of chiral HPLC are an isocratic mixture of eluent A (EtOH, in presence or absence of an amine such as triethylamine or diethylamine) and eluent B (hexane), at a flow rate of 0.8 to 150 mL/min.

### EXAMPLES

The following examples illustrate the invention but do not at all limit the scope thereof.

All temperatures are stated in ° C. Compounds are characterized by ¹H-NMR (400 MHz) or ¹³C-NMR (100 MHz) (Bruker; chemical shifts are given in ppm relative to the solvent used; multiplicities: s=singlet, d=doublet, t=triplet, q=quadruplet, quint=quintuplet, hex=hexet, hept=heptet, m=multiplet, br=broad, coupling constants are given in Hz); by LC-MS (Finnigan Navigator with HP 1100 Binary Pump and DAD, column: 4.6×50 mm, Zorbax SB-AQ, 5 μm, 120 Å, gradient: 5-95% acetonitrile in water, 1 min, with 0.04% trifluoroacetic acid, flow: 4.5 mL/min), retention times or LC-MS marked with * refer to a LC run under basic conditions, i.e. eluting with a gradient of MeCN in water containing 13 mM of ammonium hydroxide, otherwise identical conditions, t_{R} is given in min; by TLC (TLC-plates from Merck, Silica gel 60 F₂₅₄); or by melting point. Compounds are purified by preparative HPLC (columns: X-terra RP18, 50×19 mm, 5 μm or X-Bridge PrepC18, 30×75 mm, 5 μm; gradient: 10-95% acetonitrile in water containing 0.5% of formic acid) or by MPLC (Labomatic MD-80-100 pump, Linear UVIS-201 detector, column: 350×18 mm, Labogel-RP-18-5s-100, gradient: 10% methanol in water to 100% methanol). Racemates can be separated into their enantiomers by preparative HPLC (column: ChiralPaK AD 20×250 mm, 5 μm, 15% ethanol in hexane).

### Abbreviations (as Used Hereinbefore or Hereinafter):

aq. aqueous
atm atmosphere
BSA bovine serum albumin
CC column chromatography
CDI carbonyl diimidazole
dba dibenzylidene acetone
DCC dicyclohexyl carbodiimide
DCM dichloromethane
DEAD diethyl azodicarboxylate
DIPEA diisopropyl-ethylamine, Hünig's base, ethyl-diisopropylamine
DME 1,2-dimethoxyethane
DMF dimethylformamide
DMSO dimethylsulfoxide
dppf 1,1′-bis(diphenylphosphino-KP)ferrocene
EA ethyl acetate
EDC N-(3-dimethylaminopropyl)-N′-ethyl-carbodiimide
eq. equivalent(s)
Et ethyl
EtOAc ethyl acetate
Ex. example(s)
Fe(acac)₃ iron(III) acetylacetone-complex
h hour(s)
HBTU O-(benzotriazol-1-yl)-N,N,N′,N′-tetramethyluronium hexafluorophosphate
HOBt 1-hydroxybenzotriazole
HPLC high performance liquid chromatography
HV high vacuum conditions
i-Bu isobutyl
i-Pr isopropyl
KOtBu potassium tert-butoxide
LC-MS liquid chromatography—mass spectrometry
Lit. literature
mCPBA meta-chloroperbenzoic acid
Me methyl
MeOH methanol
min minute(s)
MPLC medium pressure liquid chromatography
NaOAc sodium acetate
n-BuLi n-butyl-lithium
NMP N-methylpyrrolidin-2-one
n-Pr n-propyl
OAc acetate
org. organic
Ph phenyl
PyBOP benzotriazol-1-yl-oxy-tris-pyrrolidino-phosphonium-hexafluoro-phosphate
prep. preparative
rt room temperature
sat. saturated
S1P sphingosine 1-phosphate
TBTU 2-(1H-benzotriazole-1-yl)-1,2,3,3-tetramethyluronium tetrafluoroborate
TFA trifluoroacetic acid
TFAA trifluoroacetic anhydride
THF tetrahydrofuran
TLC thin layer chromatography
t_{R} retention time
Xantphos 4,5-bis(diphenylphosphino)-9,9-dimethylxanthene

### 6-Methyl-2-propylamino-pyrimidine-4-carboxylic acid

a) To a solution of commercially available methyl-2-chloro-6-methylpyrimidine-4-carboxylate (6.00 g, 32.15 mmol) in acetonitrile (500 mL), 1M aq. solution of NaOH (48.2 mL) is added at 0° C. The mixture is stirred at 0° C. for 1 h then acidified with 25% aq. HCl (7 mL). Volatiles are evaporated and the aq. solution is extracted with ethylacetate, washed with brine, dried over Na₂SO₄, filtered and concentrated to give 2-chloro-6-methylpyrimidine-4-carboxylic acid (4.22 g) as a yellow crystalline solid; LC-MS: t_{R}=0.42 min, [M+H]⁺=172.96; ¹H NMR (D₆-DMSO): δ 2.58 (s, 3H), 7.95 (s, 1H), 14.1 (s br, 1H).
b) A solution of 2-chloro-6-methylpyrimidine-4-carboxylic acid (100 mg, 0.58 mmol) and propylamine (0.48 mL) in dioxane (1 mL) was stirred at 70° C. for 18 h. The reaction mixture is concentrated and purified by prep. HPLC (XBridge) to give 6-methyl-2-propylamino-pyrimidine-4-carboxylic acid (81 mg) as a yellow crystalline solid; LC-MS: t_{R}=0.56 min, [M+H]⁺=196.08; ¹H NMR (D₆-DMSO): δ 0.89 (t, J=7.3 Hz, 3H), 1.53 (m, 2H), 2.32 (s, 3H), 3.25 (m, 2H), 6.92 (s, 1H), 7.36 (s br, 1H), 13.2 (s br, 1H).

### 6-Methyl-2-methylamino-pyrimidine-4-carboxylic acid

The title compound is obtained as a yellow solid (230 mg) in analogy to 6-methyl-2-propylamino-pyrimidine-4-carboxylic acid starting from 2-chloro-6-methylpyrimidine-4-carboxylic acid (265 mg, 1.54 mmol) and 41% methylamine in water; LC-MS: t_{R}=0.32 min, [M+H]⁺=168.04.

### 2-Ethylamino-6-methyl-pyrimidine-4-carboxylic acid

The title compound is obtained as a yellow solid (233 mg) in analogy to 6-methyl-2-propylamino-pyrimidine-4-carboxylic acid starting from 2-chloro-6-methylpyrimidine-4-carboxylic acid (265 mg, 1.54 mmol) and 2-Methylamine in THF; LC-MS: t_{R}=0.47 min, [M+H]⁺=182.05.

### 2-Isopropylamino-6-methyl-pyrimidine-4-carboxylic acid

The title compound is obtained as a yellow solid (244 mg) in analogy to 6-methyl-2-propylamino-pyrimidine-4-carboxylic acid starting from 2-chloro-6-methylpyrimidine-4-carboxylic acid (265 mg, 1.54 mmol) and isopropylamine; LC-MS: t_{R}=0.55 min, [M+H]⁺=196.05.

### 2-Isobutylamino-6-methyl-pyrimidine-4-carboxylic acid

The title compound is obtained as a yellow solid (271 mg) in analogy to 6-methyl-2-propylamino-pyrimidine-4-carboxylic acid starting from 2-chloro-6-methylpyrimidine-4-carboxylic acid (265 mg, 1.54 mmol) and isobutylamine; LC-MS: t_{R}=0.64 min, [M+H]⁺=210.08.

### 2-Dimethylamino-6-methyl-pyrimidine-4-carboxylic acid

The title compound is obtained as a yellow solid (238 mg) in analogy to 6-methyl-2-propylamino-pyrimidine-4-carboxylic acid starting from 2-chloro-6-methylpyrimidine-4-carboxylic acid (265 mg, 1.54 mmol) and 2M dimethylamine in THF; LC-MS: t_{R}=0.44 min, [M+H]⁺=182.08.

### 2-(Ethyl-methyl-amino)-6-methyl-pyrimidine-4-carboxylic acid

The title compound is obtained as a yellow solid (235 mg) in analogy to 6-methyl-2-propylamino-pyrimidine-4-carboxylic acid starting from 2-chloro-6-methylpyrimidine-4-carboxylic acid (265 mg, 1.54 mmol) and N-ethylmethylamine; LC-MS: t_{R}=0.54 min, [M+H]⁺=196.08.

### 6-Methyl-2-(methyl-propyl-amino)-pyrimidine-4-carboxylic acid

The title compound is obtained as a brown oil (285 mg) in analogy to 6-methyl-2-propylamino-pyrimidine-4-carboxylic acid starting from 2-chloro-6-methylpyrimidine-4-carboxylic acid (300 mg, 1.74 mmol) and N-methylpropylamine; LC-MS: t_{R}=0.69 min, [M+H]⁺=210.22. ¹H NMR (CDCl₃): δ 0.97 (t, J=7.5 Hz, 3H), 1.68 (m, 2H), 2.93 (s, 3H), 3.24 (s, 3H), 3.67 (m, 2H), 7.15 (s, 1H).

### 2-Diethylamino-6-methyl-pyrimidine-4-carboxylic acid

The title compound is obtained as a yellow solid (192 mg) in analogy to 6-methyl-2-propylamino-pyrimidine-4-carboxylic acid starting from 2-chloro-6-methylpyrimidine-4-carboxylic acid (265 mg, 1.54 mmol) and diethylamine; LC-MS: t_{R}=0.64 min, [M+H]⁺=210.07.

### 2-(Isobutyl-methyl-amino)-6-methyl-pyrimidine-4-carboxylic acid

The title compound is obtained as a yellow solid (235 mg) in analogy to 6-methyl-2-propylamino-pyrimidine-4-carboxylic acid starting from 2-chloro-6-methylpyrimidine-4-carboxylic acid (300 mg, 1.74 mmol) and N-isobutylmethylamine; LC-MS: t_{R}=0.80 min, [M+H]⁺=224.21.

### 6-Methyl-2-pyrrolidin-1-yl-pyrimidine-4-carboxylic acid

The title compound is obtained as a yellow solid (200 mg) in analogy to 6-methyl-2-propylamino-pyrimidine-4-carboxylic acid starting from 2-chloro-6-methylpyrimidine-4-carboxylic acid (200 mg, 1.16 mmol) and pyrrolidine; LC-MS: t_{R}=0.50 min, [M+H]⁺=208.00.

### 2-Cyclopropylamino-6-methyl-pyrimidine-4-carboxylic acid

The title compound is obtained as a yellow solid (336 mg) in analogy to 6-methyl-2-propylamino-pyrimidine-4-carboxylic acid starting from 2-chloro-6-methylpyrimidine-4-carboxylic acid (500 mg, 2.90 mmol) and cyclopropylamine; LC-MS: t_{R}=0.44 min, [M+H]⁺=193.98. ¹H NMR (D₆-DMSO) δ 0.47 (m, 2H), 0.66 (m, 2H), 2.35 (s, 3H), 2.79 (m, 1H), 6.99 (s, 1H), 7.55 (s br, 1H).

### 2-(Cyclopropylmethyl-amino)-6-methyl-pyrimidine-4-carboxylic acid

The title compound is obtained as a yellow oil (230 mg) in analogy to 6-methyl-2-propylamino-pyrimidine-4-carboxylic acid starting from 2-chloro-6-methylpyrimidine-4-carboxylic acid (250 mg, 1.45 mmol) and cyclopropylmethylamine; LC-MS: t_{R}=0.57 min, [M+H]⁺=208.00.

### 2-(Isopropyl-methyl-amino)-6-methyl-pyrimidine-4-carboxylic acid

The title compound is obtained as a yellow oil (640 mg) in analogy to 6-methyl-2-propylamino-pyrimidine-4-carboxylic acid starting from 2-chloro-6-methylpyrimidine-4-carboxylic acid (500 mg, 2.90 mmol) and isopropylmethylamine; LC-MS: t_{R}=0.44 min, [M+H]⁺=210.31. ¹H NMR (D₆-DMSO) δ 1.15 (m, 6H), 2.34 (m, 3H), 2.96 (s, 3H), 5.09 (m, 1H), 6.94 (s, 1H), 13.2 (s br, 1H).

### 2-(Ethyl-propyl-amino)-6-methyl-pyrimidine-4-carboxylic acid

The title compound is obtained as a yellow oil (526 mg) in analogy to 6-methyl-2-propylamino-pyrimidine-4-carboxylic acid starting from 2-chloro-6-methylpyrimidine-4-carboxylic acid (500 mg, 2.90 mmol) and N-ethylpropylamine; LC-MS: t_{R}=0.61 min, [M+H]⁺=224.29. ¹H NMR (D₆-DMSO) δ 0.88 (m, J=7.3 Hz, 3H), 1.11 (t, J=6.8 Hz, 3H), 1.58 (m, 2H), 2.35 (s, 3H), 3.53 (m, 2H), 3.62 (q, J=6.8 Hz, 2H), 6.93 (s, 1H), 13.1 (s br, 1H).

### 2-(Ethyl-isopropyl-amino)-6-methyl-pyrimidine-4-carboxylic acid

The title compound is obtained as a brown oil (334 mg) in analogy to 6-methyl-2-propylamino-pyrimidine-4-carboxylic acid starting from 2-chloro-6-methylpyrimidine-4-carboxylic acid (500 mg, 2.90 mmol) and N-ethylisopropylamine; LC-MS: t_{R}=0.74 min, [M+H]⁺=224.03.

### 2-Methoxy-6-methyl-pyrimidine-4-carboxylic acid

A suspension of methyl-2-chloro-6-methylpyrimidine-4-carboxylate (5.0 g) in 2N aq. NaOH (67 mL) and MeOH (67 mL) is stirred at rt for 24 h. MeOH is evaporated and the aq. phase is acidified with 25% aq. HCl at 0° C. A beige crystalline solid crushes out. It is filtered, rinsed with water and heptane and dried to give the title compound (3.0 g); LC-MS: t_{R}=0.55 min, [M+H]⁺=169.01; ¹H NMR (D₆-DMSO): δ 2.50 (s, 3H), 3.94 (s, 3H), 7.52 (s, 1H), 13.7 (s br, 1H).

### 2-Ethoxy-6-methyl-pyrimidine-4-carboxylic acid

A solution of 2-chloro-6-methylpyrimidine-4-carboxylic acid (265 mg, 1.54 mmol) and Hünig's base (0.8 mL) in ethanol (1.79 mL) is stirred at 70° C. for 24 h. Ethanol is evaporated and the aq. phase is acidified with 25% aq. HCl at 0° C., concentrated and purified by prep. HPLC (XBridge) to give 2-ethoxy-6-methyl-pyrimidine-4-carboxylic acid (201 mg) as a yellow solid; LC-MS: t_{R}=0.63 min, [M+H]⁺=183.04.

### 6-Methyl-2-propoxy-pyrimidine-4-carboxylic acid

The title compound is obtained as a yellow solid (174 mg) in analogy to 2-ethoxy-6-methyl-amino-pyrimidine-4-carboxylic acid starting from 2-chloro-6-methylpyrimidine-4-carboxylic acid (265 mg, 1.54 mmol) and n-propanol; LC-MS: t_{R}=0.72 min, [M+H]⁺=197.06.

### 2-Isopropoxy-6-methyl-pyrimidine-4-carboxylic acid

The title compound is obtained as a yellow solid (47 mg) in analogy to 2-ethoxy-6-methyl-amino-pyrimidine-4-carboxylic acid starting from 2-chloro-6-methylpyrimidine-4-carboxylic acid (265 mg, 1.54 mmol) and isopropanol; LC-MS: t_{R}=0.71 min, [M+H]⁺=197.07.

### 2-Isobutoxy-6-methyl-pyrimidine-4-carboxylic acid

The title compound is obtained as a yellow solid (16 mg) in analogy to 2-ethoxy-6-methyl-amino-pyrimidine-4-carboxylic acid starting from 2-chloro-6-methylpyrimidine-4-carboxylic acid (265 mg, 1.54 mmol) and isobutanol; LC-MS: t_{R}=0.80 min, [M+H]⁺=211.03.

### 6-Ethyl-2-methanesulfonyl-pyrimidine-4-carboxylic acid

a) A solution of S-methylisothiourea sulfate (874 mg, 4.65 mmol) and 2,4-dioxohexanoic acid ethyl ester (800 mg, 4.65 mmol) in ethanol (4 mL) is heated up to 80° C. for 48 h. The reaction mixture is then filtered, evaporated and purified by prep. TLC (using heptane/EA 1/1 as eluent) to give 590 mg of 6-ethyl-2-methylsulfanyl-pyrimidine-4-carboxylic acid ethyl ester as a yellow oil. ¹H NMR (D₆-DMSO): δ 1.24 (t, J=7.5 Hz, 3H), 1.34 (t, J=7.0 Hz, 3H), 2.56 (s, 3H), 2.81 (q, J=7.5 Hz, 2H), 4.37 (q, J=7.0 Hz, 2H), 7.58 (s, 1H).
b) To a solution of 6-ethyl-2-methylsulfanyl-pyrimidine-4-carboxylic acid ethyl ester (590 mg, 2.61 mmol) in ethanol (12 mL) and THF (12 mL) is added 2M aq. LiOH (4 mL). The mixture is stirred for 12 h at rt before neutralizing with 1N aq. HCl. The aq. solution is extracted 3 times with ethylacetate and the combined org. extracts are evaporated to dryness to give 510 mg of 6-ethyl-2-methylsulfanyl-pyrimidine-4-carboxylic acid as a white solid; LC-MS: t_{R}=0.75 min, [M+H]⁺=199.02.
c) To a solution of 6-ethyl-2-methylsulfanyl-pyrimidine-4-carboxylic acid (510 mg, 2.57 mmol) in dichloromethane (15 mL) at 0° C., mCPBA (1.33 g, 5.40 mmol) is added. The reaction mixture is stirred for 12 h at rt, then quenched with 10% aq. Na₂S₂O₃ and extracted with dichloromethane (3×20 mL). The combined org. extracts are washed with sat. NaHCO₃, dried over MgSO₄, filtered and evaporated under reduced pressure. The crude material is then purified by prep. TLC (DCM/MeOH 4/1) to give 325 mg of the title compound as a yellow oil; LC-MS: t_{R}=0.54 min, [M+H]⁺=231.05.

### 6-Ethyl-2-methanesulfonyl-pyrimidine-4-carboxylic acid ethyl ester

To a solution of 6-ethyl-2-methylsulfanyl-pyrimidine-4-carboxylic acid ethyl ester (113 mg, 0.50 mmol) in DCM (5 mL) at 0° C., mCPBA (258 mg, 1.05 mmol) is added. The reaction mixture is stirred for 12 h at rt, then quenched with 10% aq. Na₂S₂O₃ and extracted with DCM (3×20 mL). The combined org. extracts are washed with sat. aq. NaHCO₃, dried over MgSO₄, filtered and evaporated under reduced pressure. The crude material is then purified by prep. TLC (DCM/MeOH 4/1) to give 112 mg of the title compound as a yellow oil; LC-MS: t_{R}=0.80 min, [M+H]⁺=259.07.

### 2-Methylsulfanyl-6-propyl-pyrimidine-4-carboxylic acid ethyl ester

The title compound is obtained as a yellow oil (3.35 g) in analogy to 6-ethyl-2-methylsulfanyl-pyrimidine-4-carboxylic acid ethyl ester starting from 2,4-dioxo-heptanoic acid ethyl ester (4.0 g) and S-methyl-isothiourea sulfate; LC-MS: t_{R}=0.99 min, [M+H]⁺=240.97. ¹H NMR (CDCl₃) δ 1.00 (m, 3H), 1.44 (m, 3H), 1.82 (m, 2H), 2.64 (s, 3H), 2.77 (m, 2H), 4.47 (q, J=7.3 Hz, 2H), 7.47 (s, 1H).

### 2-Methanesulfonyl-6-propyl-pyrimidine-4-carboxylic acid ethyl ester

The title compound is obtained as a yellow solid (107 mg) in analogy to 6-ethyl-2-methanesulfonyl-pyrimidine-4-carboxylic acid ethyl ester starting from 2-methylsulfanyl-6-propyl-pyrimidine-4-carboxylic acid ethyl ester; LC-MS: t_{R}=0.85 min, [M+H]⁺=272.96.

### 2-Methanesulfonyl-6-propyl-pyrimidine-4-carboxylic acid

The title compound is obtained as a yellow solid (371 mg) in analogy to 6-ethyl-2-methanesulfonyl-pyrimidine-4-carboxylic acid starting from 2-methylsulfanyl-6-propyl-pyrimidine-4-carboxylic acid ethyl ester; LC-MS: t_{R}=0.65 min, [M+H]⁺=244.95.

### 6-Isobutyl-2-methylsulfanyl-pyrimidine-4-carboxylic acid ethyl ester

The title compound is obtained as a yellow oil (2.31 g) in analogy to 6-ethyl-2-methylsulfanyl-pyrimidine-4-carboxylic acid ethyl ester starting from 6-methyl-2,4-dioxo-heptanoic acid ethyl ester (3.5 g) and S-methyl-isothiourea sulfate; LC-MS: t_{R}=1.03 min, [M+H]⁺=254.98. ¹H NMR (CDCl₃) δ 0.98 (d, J=6.8 Hz, 6H), 1.45 (t, J=7.0 Hz, 3H), 2.20 (m, 1H), 2.64 (s, 3H), 2.67 (d, J=7.0 Hz, 2H), 4.48 (q, J=7.0 Hz, 2H), 7.44 (s, 1H).

### 6-Isobutyl-2-methanesulfonyl-pyrimidine-4-carboxylic acid ethyl ester

The title compound is obtained as a yellow solid (107 mg) in analogy to 6-ethyl-2-methanesulfonyl-pyrimidine-4-carboxylic acid ethyl ester starting from 6-isobutyl-2-methylsulfanyl-pyrimidine-4-carboxylic acid ethyl ester; LC-MS: t_{R}=0.91 min, [M+H]⁺=286.97.

### 6-Isobutyl-2-methanesulfonyl-pyrimidine-4-carboxylic acid

The title compound is obtained as a beige solid (610 mg) in analogy to 6-ethyl-2-methanesulfonyl-pyrimidine-4-carboxylic acid starting from 6-isobutyl-2-methylsulfanyl-pyrimidine-4-carboxylic acid ethyl ester; LC-MS: t_{R}=0.72 min, [M+H]⁺=258.93.

### 6-Ethyl-2-ethylamino-pyrimidine-4-carboxylic acid

To a solution of 6-ethyl-2-methanesulfonyl-pyrimidine-4-carboxylic acid (325 mg, 1.41 mmol) in THF (5 mL) is added 70% ethylamine in water (2.0 mL). The mixture is stirred at 70° C. overnight. It is then evaporated to dryness and purified by prep. TLC (DCM/7N NH₃ in MeOH 4/1) to give 200 mg of 6-ethyl-2-ethylamino-pyrimidine-4-carboxylic acid as a yellow oil; LC-MS: t_{R}=0.58 min, [M+H]⁺=194.07. ¹H NMR (CDCl₃): δ 1.34 (m, 6H), 2.84 (d, J=7.5 Hz, 2H), 3.64 (m, 2H), 7.26 (s, 1H), 11.0 (s br, 1H).

### 6-Ethyl-2-methylamino-pyrimidine-4-carboxylic acid

The title compound is obtained as a yellow oil (14 mg) in analogy to 6-ethyl-2-ethylamino-pyrimidine-4-carboxylic acid starting from 6-ethyl-2-methanesulfonyl-pyrimidine-4-carboxylic acid (67 mg) and methylamine; LC-MS: t_{R}=0.48 min, [M+H]⁺=182.00.

### 6-Ethyl-2-propylamino-pyrimidine-4-carboxylic acid

The title compound is obtained as a yellow oil (12 mg) in analogy to 6-ethyl-2-ethylamino-pyrimidine-4-carboxylic acid starting from 6-ethyl-2-methanesulfonyl-pyrimidine-4-carboxylic acid (50 mg) and propylamine; LC-MS: t_{R}=0.65 min, [M+H]⁺=210.00.

### 6-Ethyl-2-isopropylamino-pyrimidine-4-carboxylic acid

The title compound is obtained as a yellow oil (10 mg) in analogy to 6-ethyl-2-ethylamino-pyrimidine-4-carboxylic acid starting from 6-ethyl-2-methanesulfonyl-pyrimidine-4-carboxylic acid (50 mg) and isopropylamine; LC-MS: t_{R}=0.64 min, [M+H]⁺=210.00.

### 6-Ethyl-2-isobutylamino-pyrimidine-4-carboxylic acid

The title compound is obtained as a yellow oil (10 mg) in analogy to 6-ethyl-2-ethylamino-pyrimidine-4-carboxylic acid starting from 6-ethyl-2-methanesulfonyl-pyrimidine-4-carboxylic acid (50 mg) and isobutylamine; LC-MS: t_{R}=0.71 min, [M+H]⁺=224.03.

### 2-Dimethylamino-6-ethyl-pyrimidine-4-carboxylic acid

The title compound is obtained as a yellow oil (20 mg) in analogy to 6-ethyl-2-ethylamino-pyrimidine-4-carboxylic acid starting from 6-ethyl-2-methanesulfonyl-pyrimidine-4-carboxylic acid (100 mg) and dimethylamine; LC-MS: t_{R}=0.57 min, [M+H]⁺=195.99.

### 3-Ethyl-2-(isobutylmethylamino)-pyrimidine-4-carboxylic acid

The title compound is obtained as a yellow oil (12 mg) in analogy to 6-ethyl-2-ethylamino-pyrimidine-4-carboxylic acid starting from 6-ethyl-2-methanesulfonyl-pyrimidine-4-carboxylic acid (50 mg) and N-methyl-isobutylamine; LC-MS: t_{R}=0.87 min, [M+H]⁺=238.06.

### 2-Methylamino-6-propyl-pyrimidine-4-carboxylic acid

A solution of 40% aq. methylamine (5 mL) and 2-methanesulfonyl-6-propyl-pyrimidine-4-carboxylic acid (150 mg, 0.614 mmol) is heated to 70° C. for 2 h. The reaction mixture is then evaporated and the crude compound is dissolved in 3N NaOH solution (10 mL). The aq. solution is washed with EtOAc (5 mL), is then adjusted to pH 3 with 25% aq. HCl and finally extracted with DCM (3×15 mL). The combined DCM extracts are dried over MgSO₄, filtered and evaporated to give 2-methylamino-6-propyl-pyrimidine-4-carboxylic acid as a beige powder (128 mg); LC-MS: t_{R}=0.56 min, [M+H]⁺=196.00.

### 2-Dimethylamino-6-propyl-pyrimidine-4-carboxylic acid

The title compound is obtained as a yellow oil (100 mg) in analogy to 2-methylamino-6-propyl-pyrimidine-4-carboxylic acid starting from 2-methanesulfonyl-6-propylpyrimidine-4-carboxylic acid (154 mg) and 40% aq. dimethylamine; LC-MS: t_{R}=0.68 min, [M+H]⁺=209.97.

### 6-Isobutyl-2-methylamino-pyrimidine-4-carboxylic acid

The title compound is obtained as a beige solid (84 mg) in analogy to 2-methylamino-6-propyl-pyrimidine-4-carboxylic acid starting from 6-isobutyl-2-methanesulfonyl-pyrimidine-4-carboxylic acid; LC-MS: t_{R}=0.63 min, [M+H]⁺=209.99.

### 2-Dimethylamino-6-isobutyl-pyrimidine-4-carboxylic acid

The title compound is obtained as a beige solid (132 mg) in analogy to 2-methylamino-6-propyl-pyrimidine-4-carboxylic acid starting from 6-isobutyl-2-methanesulfonyl-pyrimidine-4-carboxylic acid and 40% aq. dimethylamine; LC-MS: t_{R}=0.78 min, [M+H]⁺=224.03.

### 6-Isobutyl-2-isopropylamino-pyrimidine-4-carboxylic acid

The title compound is obtained as a beige solid (103 mg) in analogy to 2-methylamino-6-propyl-pyrimidine-4-carboxylic acid starting from 6-isobutyl-2-methanesulfonyl-pyrimidine-4-carboxylic acid and isopropylamine; LC-MS: t_{R}=0.77 min, [M+H]⁺=238.04.

### 2-Methoxy-6-propyl-pyrimidine-4-carboxylic acid

The title compound is obtained as a beige solid (84 mg) in analogy to 2-ethoxy-6-ethyl-pyrimidine-4-carboxylic acid starting from 2-methanesulfonyl-6-propyl-pyrimidine-4-carboxylic acid ethyl ester, KOtBu and methanol; LC-MS: t_{R}=0.71 min, [M+H]⁺=196.96.

### 6-Isobutyl-2-methoxy-pyrimidine-4-carboxylic acid

The title compound is obtained as a yellow oil (82 mg) in analogy to 2-ethoxy-6-ethyl-pyrimidine-4-carboxylic acid starting from 6-isobutyl-2-methanesulfonyl-pyrimidine-4-carboxylic acid ethyl ester, KOtBu and methanol; LC-MS: t_{R}=0.77 min, [M+H]⁺=210.96.

### 2,6-Dimethyl-isonicotinic acid isopropyl ester

a) A suspension of citrazinic acid (40 g, 0.258 mol) and tetramethylammonium chloride (29.4 g, 0.268 mol) in POCl₃ (71 mL, 0.774 mol) is heated up to 130° C. for 16 h. The mixture is then cooled down to rt and diluted with DCM (250 mL). The dark solution is added dropwise to isopropanol (1 L). After addition, the solvents are removed by distillation and the crude product is dissolved in DCM (200 mL), and added dropwise to a well-stirred 200 mL 10% NaOAc solution. The pH is kept around 7-8 by addition of 4N aq. NaOH. The layers are separated and the org. phase is washed with water (500 mL) followed with brine (150 mL) and evaporated to dryness. The dark solid is purified by distillation under HV (90° C.) to yield 2,6-dichloro-isonicotinic acid isopropyl ester as a white crystalline solid (60 g); LC-MS: t_{R}=1.03 min, [M+H]⁺=233.97. ¹H NMR (CDCl₃): δ 1.41 (d, J=6.0 Hz, 6H), 5.29 (m, 1H), 7.81 (s, 2H).
b) To a solution of 2,6-dichloro-isonicotinic acid isopropyl ester (5.20 g, 22.2 mmol) in dioxane (120 mL), Pd(dppf)Cl₂ (185 mg, 0.266 mmol) is added. 1.2M Dimethylzinc in toluene (53 mL, 66.6 mmol) is added dropwise to the mixture before it is stirred at 75° C. for 18 h under argon atmosphere. The mixture is carefully diluted with water, filtered through Celite and then extracted with EA. The combined org. extracts are dried over MgSO₄, filtered and concentrated. The crude product is purified by CC on silica gel eluting with heptane:EA 9:1 to give 2,6-dimethyl-isonicotinic acid isopropyl ester (3.30 g) as a yellow oil; LC-MS: t_{R}=0.58 min, [M+H]⁺=194.07. ¹H NMR (CDCl₃): δ 1.40 (d, J=6.3 Hz, 6H), 2.61 (s, 6H), 5.27 (m, 1H), 7.52 (s, 2H).

### 2-Ethyl-6-methylisonicotinic acid ethyl ester

a) To a solution of 2-chloro-6-methylisonicotinic acid (50 g, 291.4 mmol) in ethanol (750 mL), a few drops of concentrated sulfuric acid are added and the mixture is stirred at 75° C. for 24 h. The solvent is evaporated and the residue is dissolved in ethyl acetate (300 mL) and washed with a solution of sat. aq. NaHCO₃ (100 mL) followed with brine (2×70 mL). The org. extract is dried over MgSO₄, filtered and evaporated to give 2-chloro-6-methylisonicotinic acid ethyl ester (54.9 g) as a white solid after recrystallization from heptane; LC-MS: t_{R}=0.92 min, [M+1]⁺=200.17.
b) To a solution of 2-chloro-6-methyl-isonicotinic acid ethyl ester (15 g, 75.1 mmol) in DME (100 mL), vinylboroxine (18.1 g, 75.1 mmol) is added, followed by 2M aq. K₂CO₃ (15 mL), Pd(PPh₃)₄ (750 mg, 0.65 mmol), and PPh₃ (1.0 g, 6.17 mmol). The mixture is stirred at 100° C. for 15 h before it is cooled to rt, diluted with diethyl ether (300 mL) and washed with 1N aq. NaOH and brine. The org. extract is dried over MgSO₄, filtered and evaporated. The crude product is purified by CC on silica gel eluting with heptane:EA 4:1 to give 6-methyl-2-vinylisonicotinic acid ethyl ester (10.1 g) as a yellow oil; LC-MS: t_{R}=0.67 min, [M+1]⁺=192.07.
c) 6-Methyl-2-vinylisonicotinic acid ethyl ester (10.1 g, 52.8 mmol) is dissolved in THF (200 mL), Pd/C (300 mg, 10% Pd) is added and the mixture is stirred under 1 atm H₂ at rt for 16 h. The catalyst is filtered off and the filtrate is evaporated to give 2-ethyl-6-methyl-isonicotinic acid ethyl ester (10.0 g) as a colourless oil; LC-MS: t_{R}=0.59 min, [M+1]⁺=194.09. ¹H NMR (D₆-DMSO): δ 1.23 (t, J=7.5 Hz, 3H), 1.33 (t, J=7.0 Hz, 3H), 2.53 (s, 3H), 2.79 (q, J=7.5 Hz, 2H), 4.34 (q, J=7.0 Hz, 2H), 7.49 (s, 1H), 7.51 (s, 1H).

### 6-Methyl-2-propyl-isonicotinic acid ethyl ester

The title compound is prepared in analogy to 2-ethyl-2-methyl-isonicotinic acid ethyl ester using trans-propenyl boronic acid; colorless oil; LC-MS: t_{R}=0.65 min; [M+1]⁺=208.12. ¹H NMR (CDCl₃) δ 1.00 (t, J=7.3 Hz, 3H), 1.43 (t, J=7.0 Hz, 3H), 1.77 (m, 2H), 2.62 (s, 3H), 2.83 (m, 2H), 4.42 (q, J=7.3 Hz, 2H), 7.52 (s, 1H), 7.54 (s, 1H).

### 2-Isopropyl-6-methyl-isonicotinic acid ethyl ester

The title compound is prepared in analogy to 2-ethyl-2-methyl-isonicotinic acid ethyl ester using 2,4,6-triisopropenyl-cyclotriboroxane (prepared in analogy to a procedure given by F. Kerins, D. F. O'Shea J. Org. Chem. 67 (2002) 4968-4971); LC-MS: t_{R}=0.63 min; [M+1]⁺=208.11. ¹H NMR (CDCl₃) δ 1.24 (d, J=6.8 Hz, 6H), 1.33 (t, J=7.3 Hz, 3H), 2.53 (s, 3H), 3.08 (m, 1H), 4.34 (m, 2H), 7.49 (s, 1H), 7.51 (s, 1H).

### 2-Isobutyl-6-methyl-isonicotinic acid ethyl ester

The title compound is prepared in analogy to 2-ethyl-2-methyl-isonicotinic acid ethyl ester using 2,4,6-triisopropenyl-cyclotriboroxane (prepared in analogy to a procedure given by F. Kerins, D. F. O'Shea J. Org. Chem. 67 (2002) 4968-4971); colourless oil; LC-MS: t_{R}=0.71 min; [M+1]⁺=222.12. ¹H NMR (CDCl₃) δ 0.96 (d, J=6.8 Hz, 6H), 1.43 (t, J=7.3 Hz, 3H), 2.13 (m, 1H), 2.62 (s, 3H), 2.71 (d, J=7.3 Hz, 2H), 4.42 (q, J=7.3 Hz, 2H), 7.48 (s, 1H), 7.54 (s, 1H).

### 2,6-Dimethyl-isonicotinic acid

A solution of 2,6-dimethyl-isonicotinic acid isopropyl ester (1.81 g) in 25% aq. HCl (50 mL) is stirred at 65° C. for 24 h. The solvent is evaporated and the product is dried under HV to give the hydrochloride hydrate of the title compound (1.75 g) as a white solid; LC-MS: t_{R}=0.21 min, [M+1]⁺=152.06.

### 2-Isobutyl-6-methyl-isonicotinic acid

Hydrochloride hydrate of the title compound is obtained in analogy to 2,6-dimethyl-isonicotinic acid starting from 2-isopropyl-6-methyl-isonicotinic acid ethyl ester; white solid; LC-MS: t_{R}=0.50 min, [M+1]⁺=194.08.

### 2-Dimethylamino-6-methyl-isonicotinic acid

a) Concentrated H₂SO₄ (1.16 mL, 21.6 mmol) is added dropwise to a suspension of 2-chloro-6-methylpyridine-4-carboxylic acid (11.58 g, 67.49 mmol) in ethanol (100 mL). The reaction mixture is then stirred at 70° C. for 24 h. Sat. NaHCO₃ is added slowly to reach pH 8 and the aq. solution is extracted with EA three times. The org. extracts are collected, dried over MgSO₄, filtered and evaporated to give 2-chloro-6-methylpyridine-4-carboxylic acid ethyl ester (11.81 g) as an off white solid; LC-MS: t_{R}=0.91 min, [M+H]⁺=199.93.
b) Under argon, a solution of 2-chloro-6-methylpyridine-4-carboxylic acid ethyl ester (3.42 g 16.74 mmol), Na tert.-butylate (1.77 g, 18.4 mmol), Xantphos (967 mg, 1.67 mmol) and Pd(OAc)₂ (376 mg, 1.67 mmol) in 2 M dimethylamine in THF (20 mL) is stirred at 110° C. for 18 h. Another 10 mL of 2 M dimethylamine in THF is added and the mixture is stirred for another 24 h. The dark reaction mixture is cooled to rt, diluted with 6 N aq. HCl and extracted with diethyl ether (4 times). The org. extracts are concentrated, the residue is dissolved in 6 N aq. HCl and heated to 100° C. for 18 h. The orange suspension is concentrated, dissolved in 1 N aq. NaOH and concentrated again. The residue is dissolved in 1 N aq. NaOH and methanol and separated by MPLC on RP-C₁₈ silica gel to give 2-dimethylamino-6-methyl-isonicotinic acid (0.871 g) as a beige solid; LC-MS: t_{R}=0.44 min, [M+H]⁺=181.07.

### 2-Ethylamino-6-methyl-isonicotinic acid methyl ester

Under argon, Cs₂CO₃ (6.85 g, 21.0 mmol), Xantphos (1.39 g, 2.40 mmol) and Pd(OAc)₂ (314 mg, 1.40 mmol) and 2 Methylamine in THF (30 mL) are added to a solution of 2-chloro-6-picoline-4-carboxylic acid methyl ester (1.30 g, 7.00 mmol) in dioxane (20 mL). The reaction mixture is stirred at 90° C. for 15 h. The reaction mixture is then filtered, and concentrated under vacuum. The residue is diluted with water and extracted with EA (twice). The org. extracts are dried over MgSO₄, filtered, concentrated and purified by CC (eluting with heptane: EA 7/3) to give the title compound as a yellow oil (0.6 g); LC-MS: t_{R}=0.56 min, [M+H]⁺=195.00. ¹H NMR (CDCl₃) δ1.28 (t, J=7.0 Hz, 3H), 2.44 (s, 3H), 3.33 (m, 2H), 3.93 (s, 3H), 4.62 (m, 1H), 6.76 (m, 1H), 6.99 (s, 1H).

### 5-Ethyl-6-(isopropyl-methyl-amino) nicotinic acid

a) To a solution of 5,6-dichloronicotinic acid (10.0 g, 50.0 mmol) in THF (600 mL), triphenylphosphine (19.67 g, 75.0 mmol) and ethanol (5.55 g, 75.0 mmol) are added. The mixture is cooled to 0° C. before DEAD (32.65 g, 75.0 mmol) is added. The mixture is stirred and warmed to rt. Stirring is continued for 16 h before sat. aq. NaHCO₃ solution is added. The mixture is repeatedly extracted with EA. The combined org. extracts are dried over MgSO₄, filtered and concentrated. The crude product is purified by CC (heptane:EA 7:3) to give 5,6-dichloronicotinic acid ethyl ester (11.4 g) as a white solid; LC-MS: t_{R}=0.96 min, [M+1]⁺=220.02.
b) A mixture of 5,6-dichloronicotinic acid ethyl ester (2.91 g, 15.2 mmol) and N-isopropyl-methyl-amine (44.34 g, 60.6 mmol) is stirred in a sealed vessel at 80° C. for 48 h. The mixture is cooled to rt and concentrated. The residue is dissolved in DCM (15 mL) and washed with 1 N aq. KHSO₄ solution (2×50 mL). The washings are extracted back with DCM (50 mL). The combined org. extracts are dried over Na₂SO₄, filtered, concentrated and dried to give 5-chloro-6-(isopropyl-methyl-amino)-nicotinic acid ethyl ester (3.42 g) as a yellow oil; LC-MS: t_{R}=1.06 min, [M+1]⁺=257.11; ¹H NMR (CDCl₃): δ 1.25 (d, J=6.5 Hz, 6H), 1.39 (t, J=7.0 Hz, 3H), 2.96 (s, 3H), 4.36 (q, J=7.0 Hz, 2H), 4.55 (m, 1H), 8.09 (s, 1H), 8.72 (s, 1H).
c) To a solution of 5-chloro-6-(isopropyl-methyl-amino)-nicotinic acid ethyl ester (3.53 g, 13.7 mmol) in dioxane (60 mL), Pd(dppf) (112 mg, 0.137 mmol) is added under argon. To this mixture, diethyl zinc (10.2 g, 82.4 mmol, as a 1.1 M solution in toluene) is added dropwise. The mixture is stirred at 75° C. for 24 h before another portion of Pd(dppf) (112 mg, 0.137 mmol) and diethyl zinc (5.09 g, 41.2 mmol, as a 1.1 M solution in toluene) are added. Stirring is continued at 75° C. for 24 h. The reaction mixture is cooled to rt and carefully quenched with water. The mixture is filtered over celite and the filtrate is extracted twice with EA. The combined org. extracts are dried over MgSO₄, filtered and concentrated. The crude product is purified by CC (heptane:EA 9:1) to give 5-ethyl-6-(isopropyl-methyl-amino) nicotinic acid ethyl ester (0.855 g) as a pale yellow oil; LC-MS: t_{R}=0.78 min, [M 1]⁺=251.19; ¹H NMR (CDCl₃): δ 1.21 (d, J=6.8 Hz, 6H), 1.27 (t, J=7.3 Hz, 3H), 1.39 (t, J=7.0 Hz, 3H), 2.65 (q, J=7.3 Hz, 2H), 2.83 (s, 3H), 4.02 (m, 1H), 4.37 (q, J=7.3 Hz, 2H), 7.99 (d, J=2.3 Hz, 1H), 8.74 (d, J=2.3 Hz, 1H).
d) A solution of 5-ethyl-6-(isopropyl-methyl-amino) nicotinic acid ethyl ester (0.855 g) in 25% aq. HCl (50 mL) is stirred at 65° C. for 18 h. The solvent is evaporated and the product is dried under HV to give the hydrochloride hydrate of the title compound (2.30 g) as a white solid; LC-MS: t_{R}=0.63 min, [M+1]⁺=223.14.

### 6-Isopropoxy-5-methyl-nicotinic acid

a) To a solution of potassium tert. butylate (1.26 g, 11.3 mmol) in isopropanol (30 mL), 2,5-dibromo-3-picoline (2.89 g, 11.3 mmol) is added. The mixture is stirred at 80° C. for 15 h before another portion of potassium tert.-butylate (2.53 g, 27.5 mmol) is added. Stirring is continued at 80° C. for 24 h before the mixture is diluted with sat. aq. NaHCO₃-solution. The mixture is extracted with ether, the org. extract is dried over MgSO₄, filtered and concentrated. The crude product is purified by CC on silica gel eluting with heptane:EA 9:1 to give 5-bromo-2-isopropoxy-3-methyl-pyridine (1.24 g) as a colourless oil; LC-MS: t_{R}=1.06 min; [M+1]⁺=230.00; ¹H NMR (CDCl₃): δ 1.35 (d, J=6.3 Hz, 6H), 2.16 (s, 3H), 5.27 (hept, J=6.3 Hz, 1H), 7.48 (d, J=1.5 Hz, 1H), 8.02 (d, J=2.0 Hz, 1H).
b) A solution of 5-bromo-2-isopropoxy-3-methyl-pyridine (1.24 g, 5.39 mmol) and 2,4,6-trivinylcyclotriboroxane pyridine complex (1.27 g, 5.26 mmol) in DME (12 mL) and 2 M aq. K₂CO₃ (5 mL) is degassed and put under argon before Pd(PPh₃)₄ (112 mg, 0.097 mmol) is added. The mixture is stirred at 80° C. for 15 h before it is cooled to rt, diluted with ether (50 mL), washed with sat. aq. NaHCO₃ solution (2×30 mL), dried over MgSO₄, filtered and concentrated. The crude product is purified by CC on silica gel eluting with heptane:EA 9:1 to give 2-isopropoxy-3-methyl-5-vinyl-pyridine (703 mg) as pale yellow oil; LC-MS: t_{R}=1.01 min; [M+1]⁺=178.11.
c) To a solution of 2-isopropoxy-3-methyl-5-vinyl-pyridine (703 mg, 3.97 mmol) in acetone (80 mL), KMnO₄ (1.60 g, 10.1 mmol) is added and the mixture is stirred at rt for 18 h. The dark brown suspension is filtered and the clear, colourless filtrate is evaporated to dryness to give 6-isopropoxy-5-methyl-nicotinic acid (1.06 g, as potassium salt) as an off-white solid; LC-MS: t_{R}=0.86 min; [M+1]⁺=196.09; ¹H NMR (D₂O): δ 1.31 (d, J=6.3 Hz, 6H), 2.14 (s, 3H), 5.15 (hept, J=7.0 Hz, 1H), 7.91 (s, 1H), 8.34 (s, 1H).

### 6-Chloro-5-methyl-nicotinic acid

a) Phosphoroxychloride (183 mL, 2 mol) is heated at 90° C. and a mixture of commercially available 2-methyl-2-butennitrile (73 g, 0.9 mol) and DMF (154 mL, 2 mol) is added slowly while keeping the temperature at 100 to 110° C. The mixture is stirred at 110° C. for 15 h, cooled to rt and diluted with DCM (500 mL). The mixture is cooled at 0° C. and carefully quenched with water (500 mL). The phases are separated and the aq. phase extracted with DCM (total of 800 mL). The combined org. extracts are dried (Na₂SO₄), filtered and evaporated. The residue is crystallised from cyclohexane to provide 6-chloro-3-formyl-5-methyl-pyridine (28.3 g) as slightly yellow crystals; LC-MS: t_{R}=0.76 min, [M+1]⁺=156.14.
b) A solution of 6-chloro-3-formyl-5-methyl-pyridine (10 g, 64 mmol) in formic acid (200 mL) is cooled at 0° C. and an aq. 50% weight solution of H₂O₂ in water (9.6 mL, 360 mmol) is added at this temperature. The mixture is stirred at 0° C. for 15 h, carefully diluted with water (200 mL) and extracted with DCM (8×100 mL). The combined org. extracts are washed with 1M aq. HCl (100 mL) (check for remaining peroxide), dried (MgSO₄), filtered and evaporated. The residue is dried to give the title compound (9.56 g); LC-MS: t_{R}=0.72 min, [M+1]⁺=172.0.

### 6-Isobutyl-5-methyl-nicotinic acid

a) A solution of 6-chloro-5-methyl-nicotinic acid (13.85 g, 80.75 mmol) in dry ethanol (200 mL) containing some drops of concentrated H₂SO₄ is stirred at reflux for 2 days. The solution is cooled to rt, the solvent evaporated, the residue dissolved in EA (200 mL) and washed with a solution of sat. aq. Na₂CO₃ (2×80 mL), 1M aq. KHSO₄ (2×80 mL) and brine (50 mL). The org. phase is dried over MgSO₄, filtered and evaporated to give 6-chloro-5-methyl-nicotinic acid ethyl ester (12.65 g) as a solid; LC-MS: t_{R}=0.92 min; [M 1]⁺=200.10; ¹H NMR (CDCl₃) δ 1.43 (t, J=7.0 Hz, 3H), 2.46 (s, 3H), 4.43 (q, J=7.3 Hz, 2H), 8.16 (m, 1H), 8.84 (d, J=2.0 Hz, 1H).
b) To a solution of 6-chloro-5-methyl-nicotinic acid ethyl ester (4.98 g, 24.9 mmol), 2,4,6-tri-(2-methyl-propenyl)-cycloboroxane pyridine complex (5.74 g, 17.7 mmol, prepared in analogy to a procedure given by F. Kerins, D. F. O'Shea J. Org. Chem. 67 (2002) 4968-4971), and triphenylphosphine (1.15 g, 4.4 mmol) in DME (60 mL), a solution of 2 M aq. K₂CO₃ (20 mL) is added. The mixture is degassed and flushed with N₂ before Pd(PPh₃)₄ (460 mg, 0.4 mmol) is added. The mixture is stirred at 90° C. for 20 h before it is cooled to rt, diluted with EA (150 mL) and washed with sat. aq. NaHCO₃ (2×50 mL). The org. extract is dried over MgSO₄, filtered and evaporated. The crude product is purified by CC (SiO₂, heptane-EA) to give 5-methyl-6-(2-methyl-propenyl)-nicotinic acid ethyl ester (3.98 g) as an orange oil; LC-MS: t_{R}=0.72 min, [M+1]⁺=220.15.
c) 5-Methyl-6-(2-methyl-propenyl)-nicotinic acid ethyl ester (3.98 g, 18.2 mmol) is dissolved in THF (100 mL) and methanol (100 mL), Pd/C (500 mg, 10% Pd) is added and the mixture is stirred under 1 atm H₂ at rt for 15 h. The catalyst is filtered off and the filtrate is evaporated to give 6-isobutyl-5-methyl-nicotinic acid ethyl ester (3.76 g) as a colourless oil; LC-MS: t_{R}=0.75 min; [M+1]⁺=222.15; ¹H NMR (CDCl₃) δ 0.97 (d, J=6.8 Hz, 6H), 1.42 (t, J=7.3 Hz, 3H), 2.20 (hept, J=6.8 Hz, 1H), 2.38 (s, 3H), 2.75 (d, J=7.0 Hz, 2H), 4.41 (q, J=7.3 Hz, 2H), 8.03 (d, J=1.8 Hz, 1H), 9.00 (d, J=2.0 Hz, 1H).
d) A solution of 6-isobutyl-5-methyl-nicotinic acid ethyl ester (3.75 g, 16.95 mmol) in 12.5% aq. HCl (50 mL) is stirred at 65° C. for 24 h before the solvent is evaporated. The residue is dried under HV to give the hydrochloride salt of the title compound (3.55 g) as a white powder; LC-MS: t_{R}=0.57 min, [M+1]⁺=194.25.

### 6-Isobutyl-4-methyl-pyridine-2-carboxylic acid

a) A solution of n-BuLi (21.1 mL, 33.8 mmol, 1.6 M) in THF is cooled to −78° C. before a solution of 2,6-dichloropyridine (5.0 g, 33.8 mmol) in THF (36 mL) is added dropwise over a period of 20 min. The reaction mixture is stirred at −78° C. for 30 min, and then iodomethane (4.79 g, 33.8 mmol) is added. The mixture is stirred for 30 min before it is quenched with sat. aq. NH₄Cl solution at −78° C. The mixture is extracted with diethyl ether, the org. extract is dried over MgSO₄, filtered and concentrated. The crude product is purified by CC on silica gel eluting with heptane:EA 19:1 to give 2,6-dichloro-4-methyl-pyridine (2.34 g) as a colourless oil containing the regio isomer 2,6-dichloro-3-methyl-pyridine; LC-MS: t_{R}=0.89 min, [M+1]⁺=161.97.
b) To a solution of 2,6-dichloro-4-methyl-pyridine (2.34 g, 14.4 mmol) and 2,4,6-trivinyl-cyclotriboroxane pyridine complex (1.75 g, 7.26 mmol) in DME (27 mL), 2 M aq. K₂CO₃ solution (10 mL) is added. The mixture is degassed and put under argon before Pd(PPh₃)₄ (300 mg, 0.26 mmol) is added. The mixture is stirred at 80° C. for 3 h before it is cooled to rt, diluted with diethyl ether and washed with sat. aq. NaHCO₃ solution. The org. extract is dried over MgSO₄, filtered and concentrated. The crude product is purified by CC on silica gel eluting with heptane:EA 9:1. The thus obtained product is dissolved in EA, repeatedly washed with 5% aq. citric acid solution, dried over MgSO₄, filtered and evaporated to give 6-chloro-4-methyl-2-vinyl-pyridine (1.24 g) as a colourless oil; LC-MS: t_{R}=0.90 min, [M 1]⁺=154.03.
c) To a solution of 6-chloro-4-methyl-2-vinyl-pyridine (1.24 g, 8.06 mmol) in water (50 mL) and acetone (50 mL), KMnO₄ (6.53 g, 41.3 mmol) is added. The dark mixture becomes warm (40° C.) and is stirred at rt for 3 h before it is filtered over a sintered glass filter. The solvent of the colourless filtrate is evaporated to give crude 6-chloro-4-methyl-pyridine-2-carboxylic acid potassium salt (3.2 g) as a colourless solid; LC-MS: t_{R}=67 min, [M+1]⁺=171.99. This material is suspended in ethanol (150 mL) and H₂SO₄ (2 mL) is added until a clear solution forms. The mixture is heated to 70° C. for 18 h. The mixture is carefully diluted with sat. aq. NaHCO₃ solution until a pH of 9 is reached. The mixture is extracted three times with EA. The combined org. extracts are dried over MgSO₄, filtered and concentrated. The crude product is purified by CC on silica gel eluting with heptane:EA 3:2 to give ethyl 6-chloro-4-methyl-pyridine-2-carboxylate (500 mg) as a pale yellow oil; LC-MS: t_{R}=0.87 min; [M+1]⁺=200.04; ¹H NMR (CDCl₃): δ 1.45 (t, J=7.3 Hz, 3H), 2.45 (s, 3H), 4.48 (q, J=6.8 Hz, 2H), 7.35 (s, 1H), 7.89 (s, 1H).
d) To a solution of ethyl 6-chloro-4-methyl-pyridine-2-carboxylate (500 mg, 2.51 mmol) and 2,4,6-tris-(2-methyl-propenyl)-cyclotriboroxane pyridine complex (814 mg, 2.51 mmol) in DME (32 mL), 2 M aq. K₂CO₃ (12 mL) solution is added. The mixture is degassed and put under argon before Pd(PPh₃)₄ (52 mg, 0.045 mmol) is added. The mixture is stirred at 80° C. for 6 h before it is cooled to rt, diluted with diethyl ether (50 mL) and washed with sat. aq. NaHCO₃ (2×30 mL) solution. The org. extract is dried over MgSO₄, filtered and concentrated. The crude product is purified by CC on silica gel eluting with heptane:EA 9:1 to give 4-methyl-6-(2-methyl-propenyl)-pyridine-2-carboxylic acid ethyl ester (176 mg) as a yellow oil; ¹H NMR (CDCl₃): δ 1.45 (t, J=7.0 Hz, 3H), 1.97 (s, 3H), 2.12 (s, 3H), 2.42 (s, 3H), 4.46 (q, J=7.0 Hz, 2H), 6.41 (s, 1H), 7.17 (s, 1H), 7.75 (s, 1H).
e) To a solution of 4-methyl-6-(2-methyl-propenyl)-pyridine-2-carboxylic acid ethyl ester (175 mg, 0.80 mmol) in THF (5 mL) and ethanol (5 mL), Pd/C (50 mg, 10% Pd) is added. The mixture is stirred at 50° C. for 15 h under 1 bar of H₂. The catalyst is filtered off over celite and the solvent of the filtrate is evaporated to give 6-isobutyl-4-methyl-pyridine-2-carboxylic acid ethyl ester (174 mg) as a colourless oil; LC-MS: t_{R}=0.84 min, [M+1]⁺=222.48.
f) A solution of 6-isobutyl-4-methyl-pyridine-2-carboxylic acid ethyl ester (174 mg, 0.78 mmol) in 6 N aq. HCl (20 mL) is stirred at 65° C. for 18 h. The solvent is evaporated and the remaining residue is dried under HV to give the hydrochloride salt of the title compound as green oil; LC-MS: t_{R}=0.58 min, [M+1]⁺=194.09.

### 5-Bromo-4-methyl-pyridine-2-carboxylic acid ethyl ester

a) To a solution of 2,5-dibromo-4-picoline (9.00 g, 35.9 mmol) in DME (96 mL), 2,4,6-trivinyl-cyclotriboroxane pyridine complex (8.63 g, 35.9 mmol) and 2 N aq. K₂CO₃-solution (36 mL) is added. The mixture is degassed and put under argon before Pd(PPh₃)₄ (746 mg, 0.646 mmol) is added. The mixture is stirred at 80° C. for 15 h, before it is cooled to rt, diluted with diethyl ether (50 mL), washed with sat. aq. NaHCO₃-solution (2×30 mL), dried over MgSO₄, filtered and concentrated. The crude product is purified by CC on silica gel eluting with heptane:EA 9:1 to give 5-bromo-4-methyl-2-vinyl-pyridine (7.04 g) as a yellow oil; LC-MS: t_{R}=0.75 min; [M+1]⁺=198.22; ¹H NMR (CDCl₃): δ 2.41 (s, 3H), 5.50 (d, J=10.8 Hz, 1H), 6.21 (d, J=17.3 Hz, 1H), 6.74 (dd, J=17.3, 10.8 Hz, 1H), 7.22 (s, 1H), 8.59 (s, 1H).
b) To a solution of 5-bromo-4-methyl-2-vinyl-pyridine (7.04 g, 35.5 mmol) in acetone (280 mL) and water (280 mL), KMnO₄ (28.81 g, 71.1 mmol) is added. The dark mixture is stirred at rt for 3 days before it is filtered over a glass-filter pad. The colourless filtrate is evaporated to give crude 5-bromo-4-methyl-pyridine-2-carboxylic acid (10.9 g, as potassium salt) as a white solid; LC-MS: t_{R}=0.64 min, [M+1]⁺=215.90.
c) To a suspension of crude 5-bromo-4-methyl-pyridine-2-carboxylic acid (10.9 g, approximately 35.5 mmol) in ethanol (120 mL), H₂SO₄ (0.5 mL) is added. The mixture is stirred at 70° C. for 18 h. The pH of the clear solution is adjusted to pH 9 by adding sat. aq. NaHCO₃-solution and the mixture is extracted with diethyl ether (3×300 mL). The combined org. extracts are dried over MgSO₄, filtered and concentrated to give 5-bromo-4-methyl-pyridine-2-carboxylic acid ethyl ester (8.20 g) as a green oil; LC-MS: t_{R}=0.87 min, [M+1]⁺=243.91.

### 5-Bromo-6-methyl-pyridine-2-carboxylic acid ethyl ester

The title compound is prepared in analogy to 5-bromo-4-methyl-pyridine-2-carboxylic acid ethyl ester from 2,5-dibromo-6-picoline; LC-MS: t_{R}=0.92 min, [M+1]⁺=257.88.

### N-Hydroxy-2,6-dimethyl-isonicotinamidine

a) A solution of 2,6-dimethyl-isonicotinic acid isopropyl ester (3.03 g, 15.68 mmol) in 7N NH₃ in MeOH (200 mL) is stirred for 20 h at 60° C. The reaction mixture is then concentrated under reduced pressure to yield the crude 2,6-dimethyl-isonicotinamide as a white powder (3.0 g).
b) To a suspension of crude 2,6-dimethyl-isonicotinamide (2.45 g, 16.31 mmol) in DCM (40 mL), pyridine (6.4 mL, 65.2 mmol) is added. The mixture is cooled to 0° C. before trifluoroacetic anhydride (6.91 mL, 48.9 mmol) is added portionwise. Stirring is continued at 0° C. for 24 h before the reaction is quenched with water. The mixture is diluted with DCM, and the org. phase is separated and washed with 5% aq. citric acid solution followed by sat. aq. NaHCO₃ solution. The washings are extracted back twice with DCM. The combined org. extracts are dried over MgSO₄, filtered and concentrated. The crude product is purified by CC on silica gel eluting with heptane:EA 9:1 to give 2,6-dimethyl-isonicotinonitrile (1.58 g) as a yellow powder; LC-MS: t_{R}=0.53 min, [M+1]⁺=133.40. ¹H NMR (CDCl₃): δ 2.61 (s, 6H), 7.21 (s, 2H).
c) To an ice-cooled solution of potassium tert.-butylate (1.25 g, 11.1 mmol) in MeOH (20 mL), hydroxylamine hydrochloride (773 mg, 11.1 mmol) is added. The suspension is stirred for 30 min before 2,6-dimethyl-isonicotinonitrile (490 mg, 3.71 mmol) is added. The mixture is stirred at 60° C. for 15 h before it is filtered. The filtrate is evaporated to dryness and the resulting solid is washed with water and then dried under HV to give N-hydroxy-2,6-dimethyl-isonicotinamidine (503 mg) as a white powder; LC-MS: t_{R}=0.23 min; [M+1]⁺=166.01; ¹H NMR (D₆-DMSO): δ 2.43 (s, 6H), 5.88 (s, 2H), 7.30 (s, 2H), 9.90 (s, 1H).

### 2-Ethyl-N-hydroxy-6-methyl-isonicotinamidine

The title compound is prepared in analogy to N-hydroxy-2,6-dimethyl-isonicotinamidine from 2-ethyl-6-methylisonicotinic acid ethyl ester; white powder; LC-MS: t_{R}=0.31 min, [M+1]⁺=180.07; ¹H NMR (D₆-DMSO) δ 1.22 (t, J=7.5 Hz, 3H), 2.44 (s, 3H), 2.71 (q, J=7.5 Hz, 2H), 5.89 (s, 2H), 7.31 (s, 2H), 9.87 (s br, 1H).

### N-Hydroxy-2-isopropyl-6-methyl-isonicotinamidine

The title compound is prepared in analogy to N-hydroxy-2,6-dimethyl-isonicotinamidine starting from 2-isopropyl-6-methyl-isonicotinic acid ethyl ester; LC-MS: t_{R}=0.42 min, [M+1]⁺=194.08; ¹H NMR (D₆-DMSO): δ 1.22 (d, J=7.0 Hz, 6H), 2.44 (s, 3H), 2.91-3.02 (hept, J=7.0 Hz, 1H), 5.91 (s, 2H), 7.32 (s, 2H), 9.88 (s, 1H).

### N-Hydroxy-2-isobutyl-6-methyl-isonicotinamidine

The title compound is prepared in analogy to N-hydroxy-2,6-dimethyl-isonicotinamidine starting from 2-isobutyl-6-methyl-isonicotinic acid ethyl ester; LC-MS: t_{R}=0.67 min, [M 1]⁺=208.28; ¹H NMR (CDCl₃): δ 0.94 (d, J=6.5 Hz, 6H), 2.06-2.16 (m, 1H), 2.59 (s, 3H), 2.68 (d, J=7.0 Hz, 2H), 4.91 (s, 2H), 7.17 (s, 1H), 7.22 (s, 1H), 9.00 (s br, 1H).

### 2-Chloro-N-hydroxy-6-methyl-isonicotinamidine

The title compound is prepared in analogy to N-hydroxy-2,6-dimethyl-isonicotinamidine starting from 2-chloro-6-methyl-isonicotinic acid ethyl ester; white solid. LC-MS: t_{R}=0.54 min, [M+1]⁺=186.23; ¹H NMR (CDCl₃): δ 2.47 (s, 3H), 6.04 (s br, 2H), 7.53 (s, 1H), 7.54 (s, 1H), 10.1 (s, 1H).

### 2-Dimethylamino-N-hydroxy-6-methyl-isonicotinamidine

a) A solution of 2,6-dichloroisonicotinonitrile (2.50 g, 14.5 mmol) in 2 N Me₂NH in THF (20 mL) is stirred in a sealed vessel at 105° C. for 24 h. The dark suspension is cooled to rt, diluted with EA (200 mL), washed with water (2×50 mL) followed by sat. aq. NaHCO₃-solution (50 mL), dried over Na₂SO₄, filtered and concentrated to give crude 2-chloro-6-dimethylamino-isonicotinonitrile; LC-MS: t_{R}=0.96 min, [M+1]⁺=182.00. This material is dissolved in dioxane (100 mL) and Pd(dppf) (120 mg, 0.147 mmol) is added. To this solution, MeZnCl (5.02 g, 43.4 mmol, 2 M solution in THF) is slowly added. The mixture is stirred at rt for 30 min, then at 75° C. for 16 h. The orange suspension is cooled to rt, diluted with EA (150 mL) and washed with water (2×50 mL). The aq. washings are basified by adding aq. NaOH and the precipitate that forms is filtered off. The filtrate is extracted with DCM (3×70 mL). The combined org. extracts are dried over Na₂SO₄, filtered and concentrated. The crude product is purified by MPLC on silica gel eluting with EA containing MeOH to give 2-dimethylamino-6-methyl-isonicotinonitrile (699 mg) as a brownish oil which slowly solidifies; LC-MS: t_{R}=0.50 min, [M+1]⁺=162.05.
b) To an ice-cooled solution of potassium tert.-butylate (1.71 g, 15.2 mmol) in MeOH (50 mL), hydroxylamine hydrochloride (905 mg, 13.02 mmol) is added. The suspension is stirred for 30 min before 2-dimethylamino-6-methyl-isonicotinonitrile (699 mg, 4.34 mmol) is added. The mixture is refluxed for 2 h before it is evaporated. The residue is dissolved in a small amount of water and separated by MPLC on RP-C₁₈-silica gel to give 2-dimethylamino-N-hydroxy-6-methyl-isonicotinamidine (284 mg) as a brownish resin; LC-MS: t_{R}=0.60 min, [M+1]⁺=195.42.

### 2-(Ethyl-methyl-amino)-N-hydroxy-6-methyl-isonicotinamidine

The title compound is prepared in analogy to 2-dimethylamino-N-hydroxy-6-methyl-isonicotinamidine starting from 2,6-dichloro-isonicotinonitrile; LC-MS: t_{R}=0.45 min, [M 1]⁺=209.10; ¹H NMR (CDCl₃): δ 1.06 (t, J=6.8 Hz, 3H), 2.29 (s, 3H), 2.98 (s, 3H), 3.56 (d, J=6.8 Hz, 2H), 5.82 (s, 2H), 6.64 (s, 1H), 6.69 (s, 1H), 9.74 (s, 1H).

### N-Hydroxy-4,6-dimethyl-pyridine-2-carboxamidine

The title compound is prepared in analogy to N-hydroxy-2,6-dimethyl-isonicotinamidine starting from 4,6-dichloro-pyridine-2-carboxylic acid methyl ester (prepared from the commercially available acid); LC-MS: t_{R}=0.38 min, [M+1]⁺=166.13.

### N-Hydroxy-6-isobutyl-5-methyl-nicotinamidine

The title compound is prepared in analogy to N-hydroxy-2,6-dimethyl-isonicotinamidine starting from 6-chloro-5-methyl-nicotinic acid ethyl ester; LC-MS: t_{R}=0.55 min, [M+1]⁺=208.04.

### 2-Ethylamino-N-hydroxy-6-methyl-isonicotinamidine

The title compound is prepared in analogy to N-hydroxy-2,6-dimethyl-isonicotinamidine starting from 2-chloro-6-methyl-isonicotinic acid methyl ester; LC-MS: t_{R}=0.55 min, [M 1]⁺=208.04.

### 2-Chloro-N-hydroxy-6-methyl-pyrimidine-4-carboxamidine

a) A solution of 2-chloro-6-methylpyrimidine-4-carboxylic acid (4.0 g, 23.2 mmol) and DIPEA (5.95 mL, 34.8 mmol) in dioxane (100 mL) is cooled down to 0° C. before adding PyBOP (12.06 g, 23.2 mmol). The mixture is stirred for 5 min and 0.5M NH₃ in dioxane (93 mL, 46.4 mmol) is added. Stirring is continued for 48 h and then the reaction mixture is evaporated and purified by prep. HPLC (X-Bridge) to give 2-chloro-6-methyl-pyrimidine-4-carboxylic acid amide as a yellow solid (1.64 g); LC-MS: t_{R}=0.33 min. ¹H NMR (D₆-DMSO): δ 2.58 (s, 3H), 7.92 (s, 1H), 7.99 (s br, 1H), 8.23 (s br, 1H).
b) To a solution of 2-chloro-6-methyl-pyrimidine-4-carboxylic acid amide (1.60 g, 9.32 mmol) in DCM (160 mL), pyridine (3.0 mL, 37.30 mmol) is added. The mixture is cooled to 0° C. before trifluoroacetic anhydride (2.59 mL, 18.64 mmol) is added portionwise. Stirring is continued at 0° C. for 1 h then at rt for 6 h before the reaction is quenched with water. The mixture is diluted with DCM, and the org. phase is separated and washed with 2N aq. HCl. The aq. phase is extracted with DCM twice and the combined org. phases are washed with water followed by brine and finally are dried over MgSO₄, filtered and concentrated to give 2-chloro-6-methyl-pyrimidine-4-carbonitrile (1.36 g) as a yellow solid; LC-MS: t_{R}=0.50 min. ¹H NMR (D₆-DMSO): δ 2.58 (s, 3H), 8.16 (s, 1H).
c) To a solution of 2-chloro-6-methyl-pyrimidine-4-carbonitrile (1.25 g, 8.14 mmol) and triethylamine (3.4 mL, 24.42 mmol) in ethanol (65 mL), hydroxylamine hydrochloride (1.13 g, 16.28 mmol) is added. The reaction mixture is stirred for 1 h at rt and is then evaporated to dryness. The residue is dissolved in ethylacetate, washed with 2M aq. Na₂CO₃ followed by brine, dried over Na₂SO₄, filtered and evaporated under HV to give 2-chloro-N-hydroxy-6-methyl-pyrimidine-4-carboxamidine (1.56 g) as a yellow solid; LC-MS: t_{R}=0.34 min; [M+1]⁺=187.41.

### N-Hydroxy-6-methyl-2-propylamino-pyrimidine-4-carboxamidine

A solution of 2-chloro-N-hydroxy-6-methyl-pyrimidine-4-carboxamidine (100 mg, 0.536 mmol) and propylamine (0.5 mL) in acetonitrile (2 mL) is stirred at 70° C. overnight. The mixture is then filtered through StratoSheres PL-HCO₃ MP SPE 1 g, 1,8 mmol Na₂CO₃ cartridge and rinsed with 2 mL acetonitrile. The filtrate was concentrated and dried under HV to give the title compound (0.12 g); LC-MS: t_{R}=0.33 min; [M+1]⁺=210.30.

### N-Hydroxy-6-methyl-2-methylamino-pyrimidine-4-carboxamidine

The title compound is obtained (80 mg) in analogy to N-hydroxy-6-methyl-2-propylamino-pyrimidine-4-carboxamidine starting from 2-chloro-N-hydroxy-6-methyl-pyrimidine-4-carboxamidine and 2N methylamine in THF; LC-MS: t_{R}=0.17 min; [M+1]⁺=182.40.

### 2-Ethylamino-N-Hydroxy-6-methyl-pyrimidine-4-carboxamidine

The title compound is obtained (117 mg) in analogy to N-hydroxy-6-methyl-2-propylamino-pyrimidine-4-carboxamidine starting from 2-chloro-N-hydroxy-6-methyl-pyrimidine-4-carboxamidine and 2N ethylamine in THF; LC-MS: t_{R}=0.25 min; [M+1]⁺=196.40.

### N-Hydroxy-2-isopropylamino-6-methyl-pyrimidine-4-carboxamidine

The title compound is obtained (179 mg) in analogy to N-hydroxy-6-methyl-2-propylamino-pyrimidine-4-carboxamidine starting from 2-chloro-N-hydroxy-6-methyl-pyrimidine-4-carboxamidine and isopropylamine; LC-MS: t_{R}=0.32 min; [M+1]⁺=210.30.

### N-Hydroxy-2-isobutylamino-6-methyl-pyrimidine-4-carboxamidine

The title compound is obtained (127 mg) in analogy to N-hydroxy-6-methyl-2-propylamino-pyrimidine-4-carboxamidine starting from 2-chloro-N-hydroxy-6-methyl-pyrimidine-4-carboxamidine and isobutylamine; LC-MS: t_{R}=0.39 min; [M+1]⁺=224.30.

### 2-Dimethylamino-N-hydroxy-6-methyl-pyrimidine-4-carboxamidine

The title compound is obtained (112 mg) in analogy to N-hydroxy-6-methyl-2-propylamino-pyrimidine-4-carboxamidine starting from 2-chloro-N-hydroxy-6-methyl-pyrimidine-4-carboxamidine and 2M dimethylamine; LC-MS: t_{R}=0.28 min; [M+1]⁺=196.30.

### 2-(Ethyl-methyl-amino)-N-hydroxy-6-methyl-pyrimidine-4-carboxamidine

The title compound is obtained (139 mg) in analogy to N-hydroxy-6-methyl-2-propylamino-pyrimidine-4-carboxamidine starting from 2-chloro-N-hydroxy-6-methyl-pyrimidine-4-carboxamidine and N-ethylmethylamine; LC-MS: t_{R}=0.34 min; [M+1]⁺=210.30.

### N-Hydroxy-6-methyl-2-(methyl-propyl-amino)-pyrimidine-4-carboxamidine

The title compound is obtained (141 mg) in analogy to N-hydroxy-6-methyl-2-propylamino-pyrimidine-4-carboxamidine starting from 2-chloro-N-hydroxy-6-methyl-pyrimidine-4-carboxamidine and N-ethylpropylamine; LC-MS: t_{R}=0.42 min; [M+1]⁺=224.30.

### N-Hydroxy-2-(isopropyl-methyl-amino)-6-methyl-pyrimidine-4-carboxamidine

The title compound is obtained (154 mg) in analogy to N-hydroxy-6-methyl-2-propylamino-pyrimidine-4-carboxamidine starting from 2-chloro-N-hydroxy-6-methyl-pyrimidine-4-carboxamidine and N-isopropylmethylamine; LC-MS: t_{R}=0.39 min; [M+1]⁺=224.30.

### N-Hydroxy-2-(isobutyl-methyl-amino)-6-methyl-pyrimidine-4-carboxamidine

The title compound is obtained (153 mg) in analogy to N-hydroxy-6-methyl-2-propylamino-pyrimidine-4-carboxamidine starting from 2-chloro-N-hydroxy-6-methyl-pyrimidine-4-carboxamidine and N-isobutylmethylamine; LC-MS: t_{R}=0.49 min; [M+1]⁺=238.40.

### 2-Diethylamino-N-hydroxy-6-methyl-pyrimidine-4-carboxamidine

The title compound is obtained (125 mg) in analogy to N-hydroxy-6-methyl-2-propylamino-pyrimidine-4-carboxamidine starting from 2-chloro-N-hydroxy-6-methyl-pyrimidine-4-carboxamidine and diethylamine; LC-MS: t_{R}=0.41 min; [M+1]⁺=224.30.

### 2-(Ethyl-propyl-amino)-N-hydroxy-6-methyl-pyrimidine-4-carboxamidine

The title compound is obtained (128 mg) in analogy to N-hydroxy-6-methyl-2-propylamino-pyrimidine-4-carboxamidine starting from 2-chloro-N-hydroxy-6-methyl-pyrimidine-4-carboxamidine and N-ethylpropylamine; LC-MS: t_{R}=0.50 min; [M+1]⁺=238.30.

### 2-(Ethyl-isopropyl-amino)-N-hydroxy-6-methyl-pyrimidine-4-carboxamidine

The title compound is obtained (128 mg) in analogy to N-hydroxy-6-methyl-2-propylamino-pyrimidine-4-carboxamidine starting from 2-chloro-N-hydroxy-6-methyl-pyrimidine-4-carboxamidine and N-ethylisopropylamine; LC-MS: t_{R}=0.48 min; [M+1]⁺=238.30.

### N-Hydroxy-2-methoxy-6-methyl-pyrimidine-4-carboxamidine

Sodium hydride (54 mg, 1.34 mmol) is added to a solution of 2-chloro-N-hydroxy-6-methyl-pyrimidine-4-carboxamidine (100 mg, 0.536 mmol) in methanol. The reaction mixture is stirred at 70° C. overnight, then diluted with water and extracted with EA (twice). The org. extracts are washed with brine, dried over Na₂SO₄, then filtered and evaporated to give the title compound as a yellow solid (74 mg); LC-MS: t_{R}=0.27 min; [M+1]⁺=183.40.

### 2-Ethoxy-N-hydroxy-6-methyl-pyrimidine-4-carboxamidine

The title compound is obtained (34 mg) in analogy to N-hydroxy-2-methoxy-6-methyl-pyrimidine-4-carboxamidine starting from 2-chloro-N-hydroxy-6-methyl-pyrimidine-4-carboxamidine and ethanol; LC-MS: t_{R}=0.36 min; [M+1]⁺=197.00.

### N-Hydroxy-2-isopropoxy-6-methyl-pyrimidine-4-carboxamidine

The title compound is obtained (26 mg) in analogy to N-hydroxy-2-methoxy-6-methyl-pyrimidine-4-carboxamidine starting from 2-chloro-N-hydroxy-6-methyl-pyrimidine-4-carboxamidine and isopropanol; LC-MS: t_{R}=0.43 min; [M+1]⁺=211.30.

### N-Hydroxy-2-isobutoxy-6-methyl-pyrimidine-4-carboxamidine

The title compound is obtained (26 mg) in analogy to N-hydroxy-2-methoxy-6-methyl-pyrimidine-4-carboxamidine starting from 2-chloro-N-hydroxy-6-methyl-pyrimidine-4-carboxamidine and isobutanol; LC-MS: t_{R}=0.53 min; [M+1]⁺=225.30.

### Hydrochloride salt of 2,6-Dimethyl-isonicotinic acid hydrazide

a) To a solution of 2,6-dimethyl-isonicotinic acid HCl salt (500 mg, 2.66 mmol), t-butyl-carbazate (359 mg, 2.72 mmol) and triethylamine (0.93 mL, 6.66 mmol) in DMF, TBTU (1.05 g, 3.28 mmol) is added. The reaction mixture is stirred at rt for 5 h, and is then diluted with 1M NaOH (41 mL) and extracted with ethylacetate (3×50 mL). The combined org. phases are dried over Na₂SO₄, filtered and evaporated to give N-(2,6-dimethyl-pyridine-4-carbonyl)-hydrazinecarboxylic acid tert-butyl ester as a yellow oil; LC-MS: t_{R}=0.56 min; [M+1]⁺=265.98.*
b) N-(2,6-dimethyl-pyridine-4-carbonyl)hydrazinecarboxylic acid tert-butyl ester is dissolved in dioxane (10 mL) and cooled down to 0° C. before adding 4N HCl in dioxane (5 mL). The reaction mixture is stirred at rt for 18 h and then evaporated. The white solid is sonicated in diethylether, filtered and dried under HV to give 2,6-dimethyl-isonicotinic acid hydrazide as its HCl salt (0.70 g); LC-MS: t_{R}=0.42 min; [M+1]⁺=165.95.*

### N-Hydroxy-2-methyl-isonicotinamidine

a) A suspension of 2-methyl-pyridine-4-carboxylic acid (1.0 g, 7.29 mmol) in methanol (50 mL) and H₂SO₄ (0.5 mL) is heated to 70° C. The solid material dissolves and stirring is continued at 70° C. for 18 h. The mixture is cooled to rt, filtered, and the filtrate is evaporated. The remaining solid is washed with diethyl ether and dried to give methyl 2-methyl-pyridine-4-carboxylate; LC-MS: t_{R}=0.39 min, [M+1]⁺=152.05. This material is dissolved in 7 N NH₃ in methanol (25 mL) and the mixture is stirred in a sealed vial for 20 h at 60° C. before it is filtered. The filtrate is evaporated to give crude 2-methyl-isonicotinamide (2.12 g) as a brownish solid. To a solution of this material in DCM (25 mL), pyridine (5.24 g, 54.0 mmol) is added. The mixture is cooled to 0° C. before TFAA (8.10 g, 38.6 mmol) is added portionwise. Stirring is continued at 0° C. for 2 h before the reaction is quenched with water. The mixture is diluted with DCM and the org. phase is separated and washed with 5% aq. citric acid solution followed by sat. aq. NaHCO₃ solution. The washings are extracted back twice with DCM. The combined org. extracts are dried over MgSO₄, filtered and concentrated. The crude product is purified on prep. TLC plates with heptane:EA 4:1 to give 2-methyl-isonicotinonitrile (330 mg); LC-MS: t_{R}=0.55 min, [M+1]⁺=119.13.
b) To a solution of 2-methyl-isonicotinonitrile (330 mg, 2.79 mmol) in methanol (12 mL), hydroxylamine hydrochloride (388 mg, 5.59 mmol) and NaHCO₃ (469 mg, 5.59 mmol) is added. The mixture is stirred in a sealed vial at 60° C. for 16 h before the solvent is evaporated. The residue is dried to give N-hydroxy-2-methyl-isonicotinamidine (550 mg); LC-MS: t_{R}=0.55 min, [M+1]⁺=152.25.

### N-Hydroxy-5,6-dimethyl-pyridine-2-carboxamidine

a) Dimethyl zinc (4.58 g, 48.0 mmol) is added to a solution of 5-bromo-6-methyl-pyridine-2-carboxylic acid ethyl ester (11.7 g, 48.0 mmol) and Pd(dppf) (392 mg, 0.48 mmol) in dioxane (40 mL). The mixture becomes warm and is stirred at rt for 1 h. Another portion of dimethyl zinc (4.58 g, 48.0 mmol) is added. The mixture is stirred at 100° C. for 2 h, then at 80° C. for 72 h before it is cooled to rt, and diluted with EA (250 mL) and ice-water (150 mL). The mixture is acidified with 2 N aq. HCl, the org. phase is separated and the aq. phase is extracted with EA (3×100 mL) and DCM (2×75 mL). The combined org. extracts are dried over Na₂SO₄, filtered and concentrated. The crude product is purified by MPLC on silica gel (heptane:EA gradient) to give 5,6-dimethyl-pyridine-2-carboxylic acid ethyl ester (434 mg) as a brownish oil; LC-MS: t_{R}=0.61 min, [M+1]⁺=179.98, ¹H NMR (CDCl₃): δ 1.45 (t, J=7.0 Hz, 3H), 2.37 (s, 3H), 2.62 (s, 3H), 4.48 (q, J=7.3 Hz, 2H), 7.55 (d, J=7.8 Hz, 1H), 7.90 (d, J=7.8 Hz, 1H).
b) The title compound is prepared from the above 5,6-dimethyl-pyridine-2-carboxylic acid ethyl ester in analogy to N-hydroxy-2-methyl-isonicotinamidine; LC-MS: t_{R}=0.49 min, [M+1]⁺=166.03.

### 5-Ethyl-N-hydroxy-6-methyl-pyridine-2-carboxamidine

a) Diethyl zinc (9.78 g, 79.2 mmol) is added to a solution of 5-bromo-6-methyl-pyridine-2-carboxylic acid isopropyl ester (14.6 g, 56.5 mmol, prepared in analogy to 5-bromo-6-methyl-pyridine-2-carboxylic acid ethyl ester) and Pd(dppf) (461 mg, 0.565 mmol) in dioxane (250 mL). The mixture is stirred at 80° C. for 18 h before it is cooled to rt, diluted with ice-water (150 mL) and EA (250 mL) and acidified with 2 N aq. HCl. The org. layer is separated and the aq. phase is extracted with EA (3×100 mL) and DCM (4×100 mL). The aq. phase is neutralised by adding sat. aq. NaHCO₃ solution and is again extracted with DCM (4×75 mL). The combined org. extracts are dried over MgSO₄, filtered and concentrated. The crude product is purified by MPLC on silica gel eluting with a gradient of EA in heptane to give 5-ethyl-6-methyl-pyridine-2-carboxylic acid isopropyl ester (7.08 g) as a pale yellow oil; LC-MS: t_{R}=0.77 min, [M+1]⁺=207.99. ¹H NMR (CDCl₃): δ 1.25 (t, J=7.5 Hz, 3H), 1.41 (d, J=6.3 Hz, 6H), 2.63 (s, 3H), 2.70 (q, J=7.5 Hz, 2H), 5.30 (hept, J=6.3 Hz, 1H), 7.54 (d, J=8.0 Hz, 1H), 7.87 (d, J=8.0 Hz, 1H).
b) The title compound is prepared from the above 5-ethyl-6-methyl-pyridine-2-carboxylic acid isopropyl ester in analogy to N-hydroxy-2-hydroxymethyl-6-methyl-isonicotinamidine; LC-MS: t_{R}=0.49 min, [M+1]⁺=180.01; ¹H NMR (CDCl₃): δ 1.24 (t, J=7.5 Hz, 3H), 2.56 (s, 3H), 2.67 (q, J=7.5 Hz, 2H), 5.77 (s br, 2H), 7.46 (d, J=8.0 Hz, 1H), 7.72 (d, J=7.8 Hz, 1H), 8.25 (s br, 1H).

### N-Hydroxy-5-isobutyl-6-methyl-pyridine-2-carboxamidine

The title compound is prepared from 5-isobutyl-6-methyl-pyridine-2-carboxylic acid ethyl ester in analogy to N-hydroxy-2-methyl-isonicotinamidine; LC-MS: t_{R}=0.72 min, [M+1]⁺=208.52; ¹H NMR (CD₃OD): δ 0.96 (d, J=6.5 Hz, 6H), 1.86-1.97 (m, 1H), 2.54-2.58 (m, 5H), 7.49 (d, J=8.0 Hz, 1H), 7.62 (d, J=8.0 Hz, 1H).

### N-Hydroxy-4,5-dimethyl-pyridine-2-carboxamidine

a) Trimethylboroxine (2.84 g, 22.6 mmol), Cs₂CO₃ (9.58 g, 29.4 mmol) and tri-tert.butyl phosphine (183 mg, 905 μmol) is added to a solution of 5-bromo-4-methyl-pyridine-2-carboxylic acid ethyl ester (5.52 g, 22.6 mmol) in dioxane (100 mL). The mixture is degassed and put under argon before Pd₂(dba)₃ (414 mg, 452 μmol) is added. The grey suspension is stirred at 100° C. for 18 h. The mixture is filtered and another portion of trimethylboroxine (2.84 g, 22.6 mmol), Cs₂CO₃ (9.58 g, 29.4 mmol), Pd₂(dba)₃ (414 mg, 452 μmol) and tri-tert.butyl phosphine (183 mg, 905 μmol) is added to the filtrate. The mixture is stirred at 100° C. for 72 h before it is again filtered. The filtrate is concentrated, diluted with DCM and washed with sat. Na₂CO₃ solution (2×25 mL) followed by brine (2×25 mL). The org. extract is dried over MgSO₄, filtered and concentrated to give crude 5,6-dimethyl-pyridine-2-carboxilic acid ethyl ester; LC-MS: t_{R}=0.57 min, [M+1]⁺=166.04.
b) The title compound is prepared from the above 5,6-dimethyl-pyridine-2-carboxilic acid ethyl ester in analogy to N-hydroxy-2-methyl-isonicotinamidine; LC-MS: t_{R}=0.48 min, [M+1]⁺=166.05; ¹H NMR (CD₃OD): δ 2.31 (s, 3H), 2.33 (s, 3H), 7.66 (s, 1H), 8.29 (s, 1H).

### 5-Ethyl-N-hydroxy-4-methyl-pyridine-2-carboxamidine

The title compound is prepared from 5-bromo-4-methyl-pyridine-2-carboxylic acid ethyl ester in analogy to 5-ethyl-N-hydroxy-6-methyl-pyridine-2-carboxamidine; LC-MS: t_{R}=0.54 min, [M+1]⁺=180.01; ¹H NMR (CDCl₃): δ 1.25 (t, J=7.5 Hz, 3H), 2.35 (s, 3H), 2.69 (q, J=7.5 Hz, 2H), 5.77 (s br, 2H), 6.44 (s br, 1H), 7.75 (s, 1H), 8.32 (s, 1H).

### N-Hydroxy-6-isobutyl-4-methyl-pyridine-2-carboxamidine

The title compound is prepared from 6-isobutyl-4-methyl-pyridine-2-carboxylic acid in analogy to N-hydroxy-2-methyl-isonicotinamidine; LC-MS: t_{R}=0.63 min, [M+1]⁺=208.29.

### N-Hydroxy-2-methoxy-6-methyl-isonicotinamidine

a) Sulfuric acid (1 mL) is added to a suspension of 2-chloro-6-methoxy-isonicotinic acid (4.16 g, 22.2 mmol) in ethanol (20 mL). The clear solution is stirred at 70° C. for 18 h. The mixture is neutralised by adding sat. aq. NaHCO₃ solution and then extracted three times with EA (3×250 mL). The combined org. extracts are dried over MgSO₄, filtered, concentrated and dried to give 2-chloro-6-methoxy-isonicotinic acid ethyl ester (4.32 g) as a white solid; LC-MS: t_{R}=1.00 min, [M+1]⁺=215.89.
b) Under argon, dimethyl zinc (14.26 g, 149 mmol, 124 mL of a 1.2 M solution in toluene) is added dropwise to a solution of 2-chloro-6-methoxy-isonicotinic acid ethyl ester (5.37 g, 24.9 mmol) and Pd(dppf) (203 mg, 0.249 mmol) in dioxane (120 mL). The mixture is heated to 75° C. and stirred for 18 h before it is cooled again to rt. The reaction is quenched by carefully adding water. The mixture is diluted further with water, filtered over celite and the filtrate is extracted with EA (2×250 mL). The combined org. extracts are dried over MgSO₄, filtered and concentrated. The crude product is purified by CC on silica gel eluting with heptane:EA 4:1 to give 2-methoxy-6-methyl-isonicotinic acid ethyl ester (4.10 g) as a colourless oil; LC-MS: t_{R}=0.92 min, [M+1]⁺=195.93. ¹H NMR (CDCl₃): δ 1.41 (t, J=7.3 Hz, 3H), 2.52 (s, 3H), 3.97 (s, 3H), 4.39 (q, J=7.3 Hz, 2H), 7.12 (s, 1H), 7.28 (s, 1H).
c) The title compound is prepared in analogy to N-hydroxy-2-methyl-nicotinamidine from the above 2-methoxy-6-methyl-isonicotinic acid ethyl ester; LC-MS: t_{R}=0.43 min, [M+1]⁺=181.96. ¹H NMR (CDCl₃): δ 2.49 (s, 3H), 3.95 (s, 3H), 4.89 (s, 2H), 6.75 (s, 1H), 6.98 (s, 1H), 8.03 (s br, 1H).

### General Method for the Preparation of 5-Pyrimidin-4-yl-[1,2,4]oxadiazole derivatives

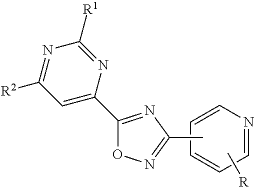

To a solution of the appropriate pyrimidine carboxylic acid (1 eq.) and DIPEA (2 eq.) in DMF is added HOBt (1.2 eq.) and EDC (1.1 eq.) or TBTU (1.2 eq.). The mixture is stirred for 15 min at rt. The appropriate N-hydroxy pyridine-carboxamidine (1 eq.) is added and stirring is continued for 1 to 8 h at rt. Formation of the amide bond is monitored by LC-MS. Upon complete conversion, the reaction is heated up to 85° C. and stirred for 24 to 48 h. Cyclization to the oxadiazole is monitored by LC-MS. The mixture is evaporated and the crude product is purified by either CC on silica gel, chromatography on prep. TLC plates, or by HPLC to give the desired 5-pyrimidin-4-yl-[1,2,4]oxadiazole derivatives in 22-82% yield.

### Examples 1 to 57

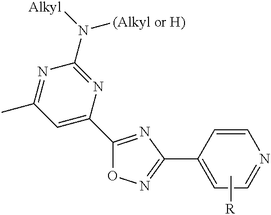

Following the general method for the preparation of 5-pyrimidin-4-yl-[1,2,4]oxadiazole derivatives, the following examples are prepared:

| | Pyrimidine | Pyridine | LC-MS | | Amount |
|---|---|---|---|---|---|
| Ex. No. | (as acid) | (as hydroxyamidine) | t_{R} [min] [M + H]⁺ | | Form |
| 1 | 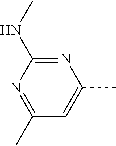 | 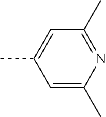 | 0.72 | 297.15 | 4.6 mg |
| 2 | 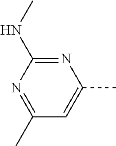 | 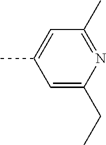 | 0.76 | 311.18 | 5 mg |
| 3 | 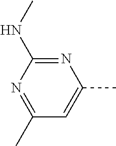 | 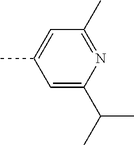 | 0.79 | 325.22 | 5 mg |
| 4 | 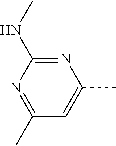 | 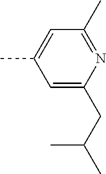 | 0.83 | 339.15 | 5 mg |
| 5 | 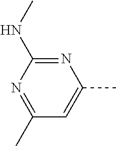 | 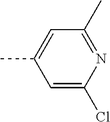 | 1.03 | 317.1 | 2.5 mg |
| 6 | 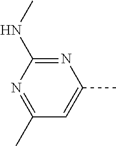 | 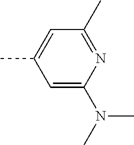 | 0.76 | 326.14 | 3.1 mg |
| 7 | 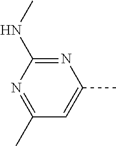 | 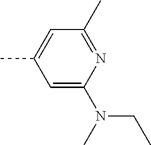 | 0.78 | 340.14 | 4.5 mg |
| 8 | 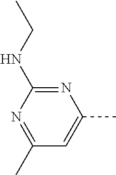 | 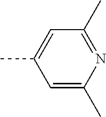 | 0.77 | 311.14 | 60 mg white crystalline solid |
| 9 | 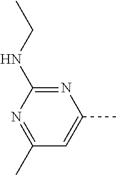 | 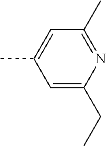 | 0.80 | 325.21 | 5 mg |
| 10 | 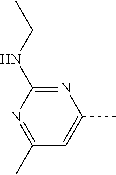 | 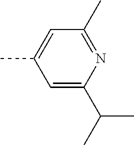 | 0.84 | 339.14 | 5 mg |
| 11 | 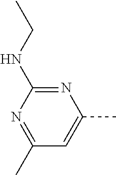 | 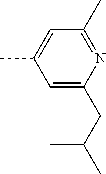 | 0.88 | 352.54 | 5 mg |
| 12 | 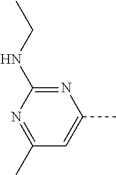 | 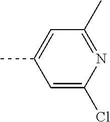 | 1.08 | 331.06 | 2.5 mg |
| 13 | 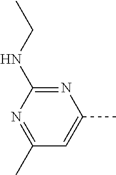 | 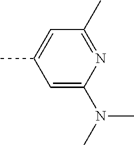 | 0.80 | 340.14 | 3.3 mg |
| 14 | 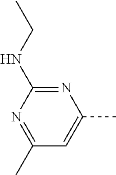 | 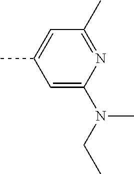 | 0.83 | 354.33 | 5.2 mg |
| 15 | 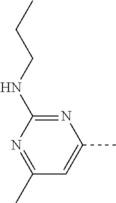 | 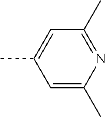 | 0.81 | 325.2 | 71 mg white crystalline solid |
| 16 | 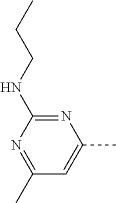 | 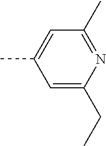 | 0.85 | 339.14 | 5.4 mg |
| 17 | 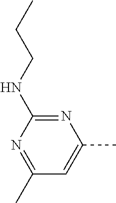 | 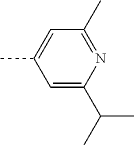 | 0.88 | 352.56 | 5.1 mg |
| 18 | 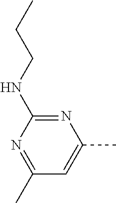 | 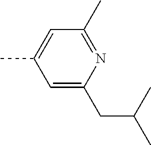 | 0.91 | 367.16 | 5.7 mg |
| 19 | 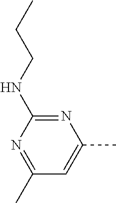 | 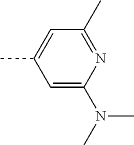 | 0.84 | 354.32 | 3.1 mg |
| 20 | 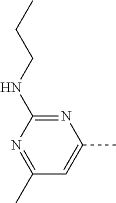 | 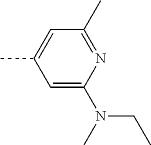 | 0.87 | 368.11 | 4.8 mg |
| 21 | 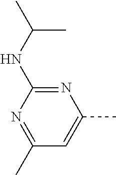 | 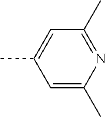 | 0.81 | 325.16 | 59 mg white cystalline solid |
| 22 | 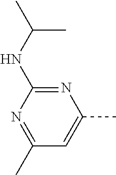 | 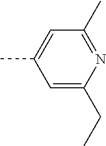 | 0.85 | 339.13 | 5.1 mg |
| 23 | 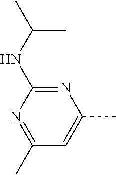 | 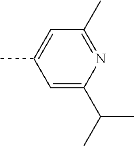 | 0.88 | 352.55 | 5.1 mg |
| 24 | 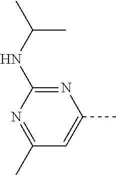 | 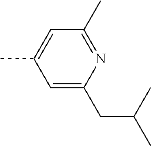 | 0.91 | 367.11 | 5.4 mg |
| 25 | 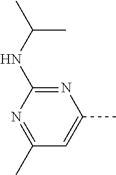 | 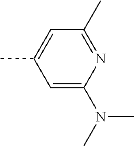 | 0.84 | 354.32 | 3.8 mg |
| 26 | 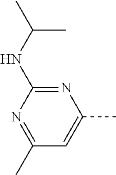 | 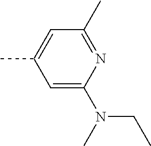 | 0.86 | 368.16 | 5.1 mg |
| 27 | 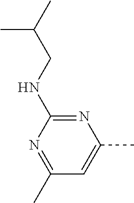 | 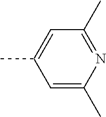 | 0.85 | 339.15 | 4.6 mg |
| 28 | 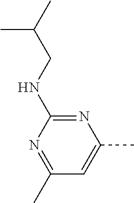 | 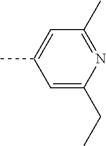 | 0.89 | 352.54 | 4.9 mg |
| 29 | 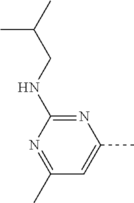 | 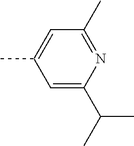 | 0.92 | 367.1 | 4.8 mg |
| 30 | 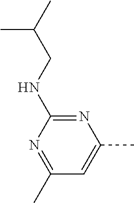 | 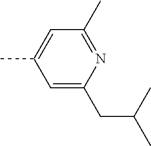 | 0.95 | 381.1 | 5.2 mg |
| 31 | 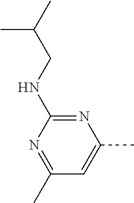 | 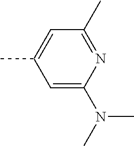 | 0.88 | 368.1 | 3.9 mg |
| 32 | 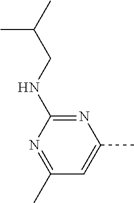 | 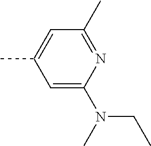 | 0.90 | 382.13 | 5.6 mg |
| 33 | 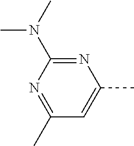 | 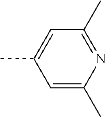 | 0.82 | 311.16 | 5.3 mg |
| 34 | 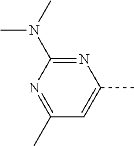 | 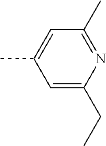 | 0.85 | 325.22 | 5.2 mg |
| 35 | 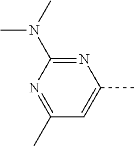 | 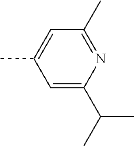 | 0.89 | 339.15 | 5.1 mg |
| 36 | 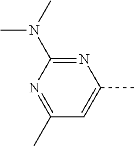 | 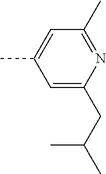 | 0.93 | 352.49 | 5.4 mg |
| 37 | 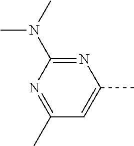 | 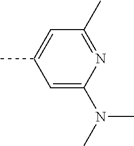 | 0.85 | 340.1 | 2.4 mg |
| 38 | 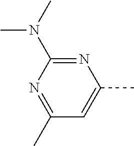 | 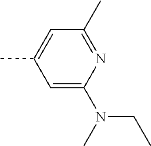 | 0.88 | 354.33 | 4.9 mg |
| 39 | 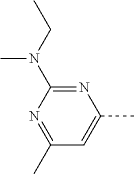 | 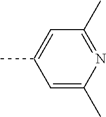 | 0.87 | 325.24 | 4.8 mg |
| 40 | 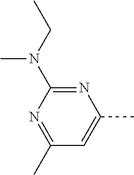 | 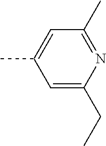 | 0.90 | 339.13 | 4.7 mg |
| 41 | 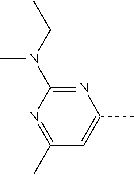 | 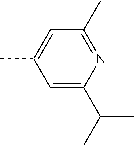 | 0.94 | 352.58 | 4.5 mg |
| 42 | 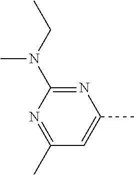 | 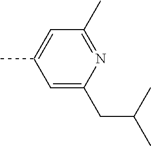 | 0.97 | 367.1 | 5.2 mg |
| 43 | 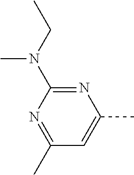 | 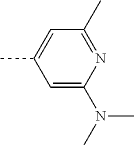 | 0.89 | 354.31 | 2.5 mg |
| 44 | 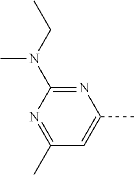 | 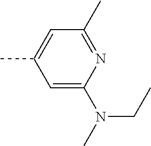 | 0.92 | 368.11 | 5.8 mg |
| 45 | 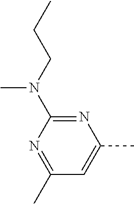 | 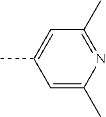 | 0.91 | 339.13 | 4.3 mg |
| 46 | 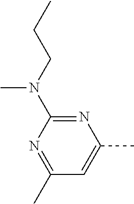 | 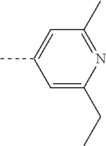 | 0.94 | 352.55 | 4.2 mg |
| 47 | 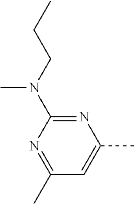 | 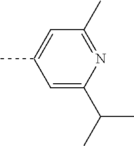 | 0.98 | 367.11 | 4.4 mg |
| 48 | 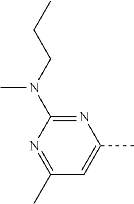 | 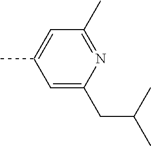 | 1.0 | 381.16 | 4.6 mg |
| 49 | 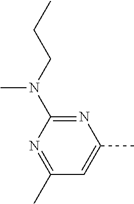 | 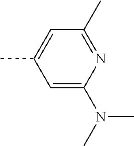 | 0.93 | 368.11 | 2.4 mg |
| 50 | 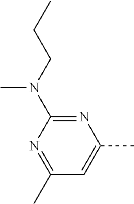 | 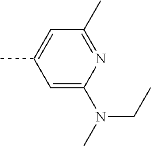 | 0.96 | 382.1 | 3.7 mg |
| 51 | 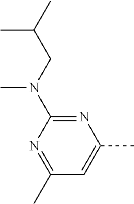 | 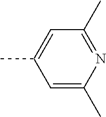 | 0.94 | 352.53 | 72 mg yellow crystalline solid |
| 52 | 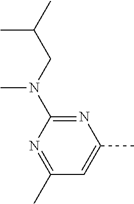 | 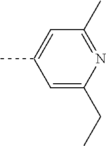 | 0.98 | 367.11 | 7.9 mg |
| 53 | 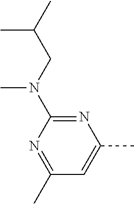 | 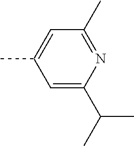 | 1.01 | 381.1 | 8.8 mg |
| 54 | 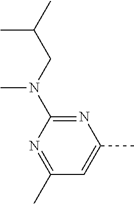 | 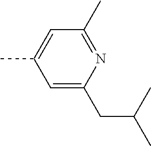 | 1.03 | 395.18 | 9.3 mg |
| 55 | 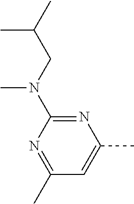 | 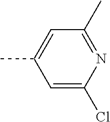 | 1.27 | 373.03 | 4.5 mg |
| 56 | 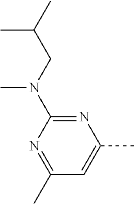 | 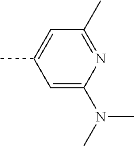 | 0.96 | 382.09 | 6.1 mg |
| 57 | 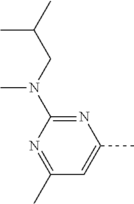 | 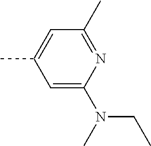 | 0.99 | 396.13 | 7.2 mg |

### Example 8

¹H NMR (D₆-DMSO): δ 1.17 (t, J=7.0 Hz, 3H), 2.42 (s, 3H), 2.56 (s, 6H), 3.38 (m, 2H), 7.30 (s, 1H), 7.68 (m, 3H).

### Example 15

¹H NMR (D₆-DMSO): δ 0.92 (t, J=7.0 Hz, 3H), 1.59 (m, 2H), 2.42 (s, 3H), 2.56 (s, 6H), 3.30 (m, 2H), 7.30 (s, 1H), 7.69 (m, 3H).

### Example 21

¹H NMR (D₆-DMSO): δ 1.19 (d, J=6.3 Hz, 6H), 2.42 (s, 3H), 2.56 (s, 6H), 4.16 (m, 1H), 7.29 (s, 1H), 7.58 (s br, 1H), 7.68 (s, 2H).

### Example 51

¹H NMR (D₆-DMSO): δ 0.90 (d, J=6.5 Hz, 6H), 2.12 (m, 1H), 2.44 (s, 3H), 2.56 (s, 6H), 3.19 (s, 3H), 3.32 (s, 2H), 3.53 (d, J=7.3 Hz, 2H), 7.31 (s, 1H), 7.68 (s, 2H).

### Examples 58 to 78

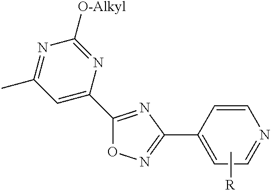

Following the general method for the preparation of 5-pyrimidin-4-yl-[1,2,4]oxadiazole derivatives, the following examples are prepared:

| | | | LC-MS | | |
|---|---|---|---|---|---|
| Ex. | Pyrimidine | Pyridine | t_{R} | [M + | |
| No. | (as acid) | (as hydroxyamidine) | [min] | H]⁺ | Amount |
| 58 | 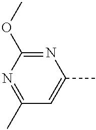 | 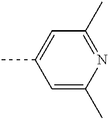 | 0.73 | 298.10 | 6.1 mg |
| 59 | 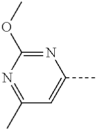 | 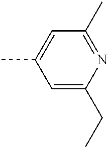 | 0.77 | 312.14 | 5.8 mg |
| 60 | 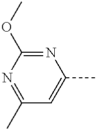 | 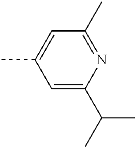 | 0.81 | 326.18 | 5.8 mg |
| 61 | 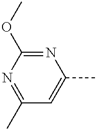 | 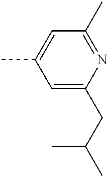 | 0.84 | 340.11 | 6.3 mg |
| 62 | 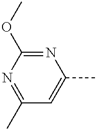 | 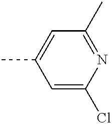 | 1.05 | 318.01 | 2.7 mg |
| 63 | 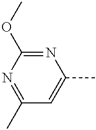 | 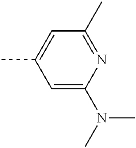 | 0.77 | 327.03 | 2.5 mg |
| 64 | 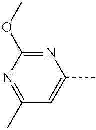 | 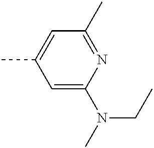 | 0.79 | 341.12 | 4.6 mg |
| 65 | 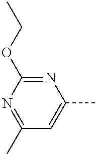 | 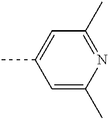 | 0.78 | 312.15 | 5.9 mg |
| 66 | 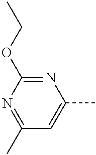 | 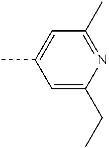 | 0.81 | 326.17 | 5.6 mg |
| 67 | 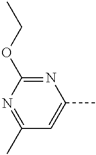 | 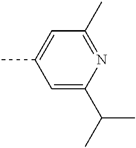 | 0.85 | 340.12 | 6.0 mg |
| 68 | 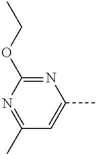 | 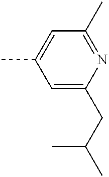 | 0.89 | 354.31 | 6.1 mg |
| 69 | 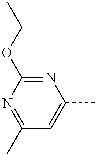 | 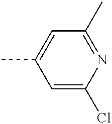 | 1.1 | 332.05 | 2.3 mg |
| 70 | 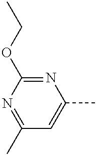 | 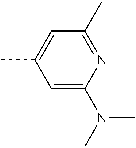 | 0.81 | 341.13 | 2.4 mg |
| 71 | 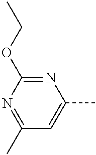 | 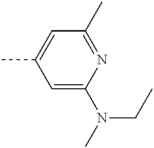 | 0.83 | 355.09 | 4.3 mg |
| 72 | 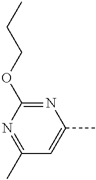 | 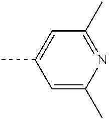 | 0.82 | 326.15 | 6.3 mg |
| 73 | 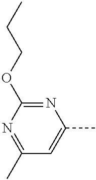 | 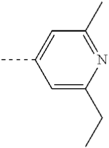 | 0.86 | 340.12 | 6.0 mg |
| 74 | 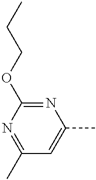 | 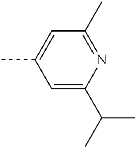 | 0.90 | 354.29 | 6 mg |
| 75 | 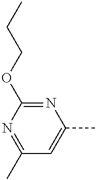 | 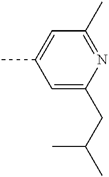 | 0.93 | 368.09 | 6.3 mg |
| 76 | 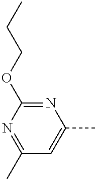 | 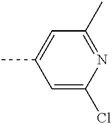 | 1.15 | 346.04 | 2.9 mg |
| 77 | 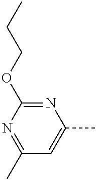 | 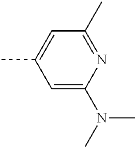 | 0.85 | 355.09 | 2.7 mg |
| 78 | 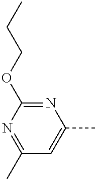 | 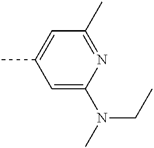 | 0.88 | 369.09 | 5.5 mg |

### Example 79

### {4-[3-(2,6-Dimethyl-pyridin-4-yl)-[1,2,4]oxadiazol-5-yl]-6-ethyl-pyrimidin-2-yl}-ethyl-amine

The title compound was prepared from 6-ethyl-2-ethylamino-pyrimidine-4-carboxylic acid and N-hydroxy-2,6-dimethyl-isonicotinamidine using the general procedure; LC-MS: t_{R}=0.75 min, [M+1]⁺=325.16; ¹H NMR (CDCl₃) δ 1.30 (t, J=7.3 Hz, 3H), 1.36 (t, J=7.8 Hz, 3H), 2.65 (s, 6H), 2.77 (q, J=7.3 Hz, 2H), 3.60 (m, 2H), 5.36 (s br, 1H), 7.32 (s, 1H), 7.75 (s, 2H).

### Example 80

### {4-[3-(4,6-Dimethyl-pyridin-2-yl)-[1,2,4]oxadiazol-5-yl]-6-methyl-pyrimidin-2-yl}-propyl-amine

The title compound was prepared from 2-propylamino-6-methyl-pyrimidine-4-carboxylic acid and N-hydroxy-4,6-dimethyl-pyridine-2-carboxamidine using the general procedure; LC-MS: t_{R}=0.96 min, [M+1]⁺=325.19; ¹H NMR (CDCl₃) δ 1.01 (t, J=7.5 Hz, 3H), 1.67 (m, 2H), 2.42 (s, 3H), 2.47 (s, 3H), 2.66 (s, 3H), 3.50 (m, 2H), 5.53 (m, 1H), 7.15 (s, 1H), 7.38 (s, 1H), 7.90 (s, 1H).

### General Methods for the Preparation of 3-Pyrimidin-4-yl-[1,2,4]oxadiazole derivatives

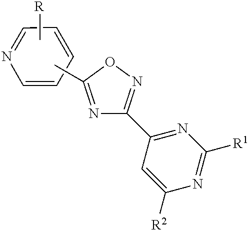

### Method A

To a solution of the appropriate pyridine carboxylic acid (1 eq.) and DIPEA (2 eq.) in DMF is added HOBt (1.2 eq.) and EDC (1.1 eq.) or TBTU (1.2 eq.). The mixture is stirred for 15 min at rt. 2-Chloro-N-hydroxy-6-methyl-pyrimidine-4-carboxamidine (1 eq.) is added and stirring is continued for 1 to 8 h at rt. Formation of the amide bond is monitored by LC-MS. Upon complete conversion, the reaction is heated up to 85° C. and stirred for 24 to 48 h. Cyclization to the oxadiazole is monitored by LC-MS. The mixture is evaporated and the crude product is purified by either CC on silica gel, chromatography on prep. TLC plates, or by HPLC to give the desired chloropyrimidyl-pyridyl-[1,2,4]oxadiazoles in 15% yield. The compound is then dissolved in acetonitrile and reacted with an excess of the appropriate amine or alcohol at temperatures ranging from rt to 60° C. (for the reaction of alcohol NaH is used). The mixture is evaporated and the crude product is purified by either CC on silica gel, chromatography on prep. TLC plates, or by HPLC to give the desired compounds in a 22-74% yield.

### Method B

To a solution of the appropriate pyridine carboxylic acid (1 eq.) and DIPEA (2 eq.) in DMF is added HOBt (1.2 eq.) and EDC (1.1 eq.) or TBTU (1.2 eq.). The mixture is stirred for 15 min at rt. The appropriate N-hydroxy pyrimidine-carboxamidine (1 eq.) is added and stirring is continued for 1 to 8 h at rt. Formation of the amide bond is monitored by LC-MS. Upon complete conversion, the reaction is heated up to 85° C. and stirred for 24 to 48 h. Cyclization to the oxadiazole is monitored by LC-MS. The mixture is evaporated and the crude product is purified by either CC on silica gel, chromatography on prep. TLC plates, or by HPLC to give the desired 3-pyrimidin-4-yl-[1,2,4]oxadiazole derivatives.

### Examples 81 to 98

Using Method A, the following 3-pyrimidin-4-yl-[1,2,4]oxadiazole derivatives are prepared:

| Ex. | Pyridine | Pyrimidine | LC-MS | | Amount |
|---|---|---|---|---|---|
| No. | (as acid) | (as hydroxyamidine) | t_{R} [min] | [M + H]⁺ | Form |
| 81 | 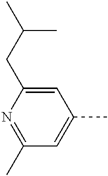 | 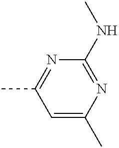 | 0.69 | 339.26 | 3.8 mg white solid |
| 82 | 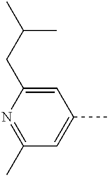 | 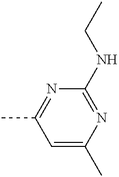 | 0.77 | 353.33 | 5.3 mg white solid |
| 83 | 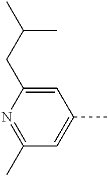 | 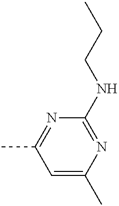 | 0.84 | 367.32 | 5.9 mg white solid |
| 84 | 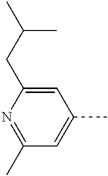 | 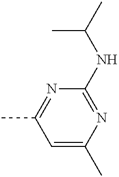 | 0.84 | 367.27 | 5.7 mg white solid |
| 85 | 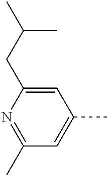 | 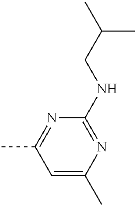 | 0.90 | 381.30 | 6.2 mg white solid |
| 86 | 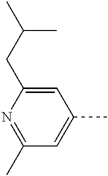 | 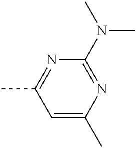 | 0.87 | 353.26 | 2.9 mg white solid |
| 87 | 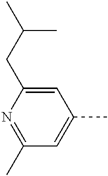 | 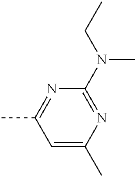 | 0.95 | 367.30 | 5.9 mg white solid |
| 88 | 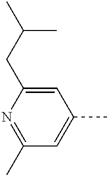 | 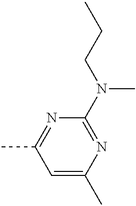 | 1.02 | 381.29 | 5.9 mg white solid |
| 89 | 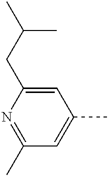 | 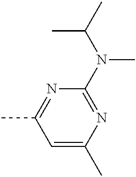 | 1.02 | 381.32 | 5.6 mg white solid |
| 90 | 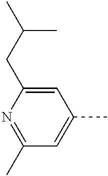 | 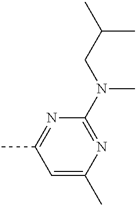 | 1.08 | 395.32 | 6.1 mg white solid |
| 91 | 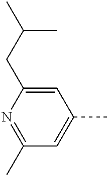 | 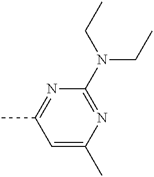 | 1.03 | 381.03 | 5.5 mg white solid |
| 92 | 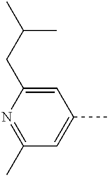 | 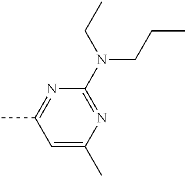 | 1.09 | 395.31 | 5.9 mg white solid |
| 93 | 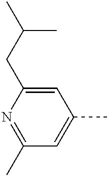 | 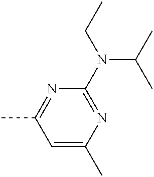 | 1.09 | 395.32 | 2.6 mg white solid |
| 94 | 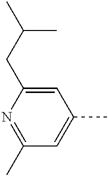 | 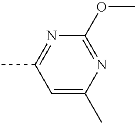 | 0.75 | 340.27 | 5.4 mg white solid |
| 95 | 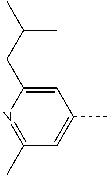 | 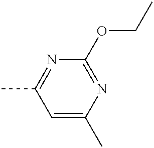 | 0.83 | 353.82 | 4.9 mg white solid |
| 96 | 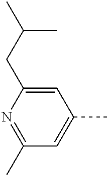 | 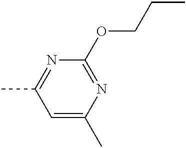 | 0.90 | 368.28 | 5.7 mg white solid |
| 97 | 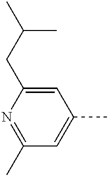 | 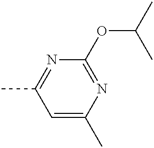 | 0.89 | 368.29 | 3.8 mg white solid |
| 98 | 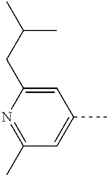 | 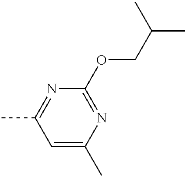 | 0.96 | 382.29 | 6.0 mg white solid |

### Example 82

¹H NMR (D₆-DMSO): δ 0.93 (d, J=6.8 Hz, 6H), 1.16 (t, J=7.3 Hz, 3H), 2.11 (m, 1H), 2.40 (s, 3H), 2.60 (s, 3H), 2.72 (d, J=7.3 Hz, 2H), 3.37 (m, 2H), 7.18 (s, 1H), 7.52 (s br, 1H), 7.72 (s, 1H), 7.80 (s, 1H).

### Example 83

¹H NMR (D₆-DMSO): δ 0.92 (m, 9H), 1.58 (m, 2H), 2.11 (m, 1H), 2.40 (s, 3H), 2.60 (s, 3H), 2.73 (d, J=7.3 Hz, 2H), 3.31 (m, 2H), 7.17 (s, 1H), 7.53 (s br, 1H), 7.72 (s, 1H), 7.79 (s, 1H).

### Example 85

¹H NMR (D₇-DMF): δ 1.34 (m, 12H), 2.33 (m, 1H), 2.53 (m, 1H), 2.81 (s, 3H), 3.02 (s, 3H), 3.14 (d, J=7.0 Hz, 2H), 3.59 (t, J=6.3 Hz, 2H), 7.58 (s, 1H), 8.00 (s br, 1H), 8.14 (s, 1H), 8.21 (s, 1H).

### Example 87

¹H NMR (D₆-DMSO): δ 0.93 (d, J=6.5 Hz, 6H), 1.15 (t, J=7.0 Hz, 3H), 2.12 (m, 1H), 2.43 (s, 3H), 2.61 (s, 3H), 2.73 (d, J=7.0 Hz, 2H), 3.16 (s, 3H), 3.73 (q, J=7.0 Hz, 2H), 7.19 (s, 1H), 7.73 (s, 1H), 7.80 (s, 1H).

### Example 89

¹H NMR (D₆-DMSO): δ 0.93 (d, J=6.5 Hz, 6H), 1.18 (d, J=6.8 Hz, 6H), 2.12 (m, 1H), 2.43 (s, 3H), 2.61 (s, 3H), 2.73 (d, J=7.3 Hz, 2H), 3.02 (s, 3H), 5.14 (m, 1H), 7.18 (s, 1H), 7.73 (s, 1H), 7.80 (s, 1H).

### Example 91

¹H NMR (D₆-DMSO): δ 0.93 (d, J=6.8 Hz, 6H), 1.17 (t, J=6.8 Hz, 6H), 2.12 (m, 1H), 2.42 (s, 3H), 2.61 (s, 3H), 2.73 (d, J=7.3 Hz, 2H), 3.67 (q, J=7.3 Hz, 4H), 7.17 (s, 1H), 7.72 (s, 1H), 7.79 (s, 1H).

### Example 94

¹H NMR (D₆-DMSO): δ 0.93 (d, J=6.5 Hz, 6H), 2.12 (m, 1H), 2.57 (s, 3H), 2.61 (s, 3H), 2.73 (d, J=7.3 Hz, 2H), 4.01 (s, 3H), 7.75 (s, 1H), 7.77 (s, 1H), 7.79 (s, 1H).

### General Method for the Preparation of 2-Pyrimidin-4-yl-[1,3,4]oxadiazole derivatives

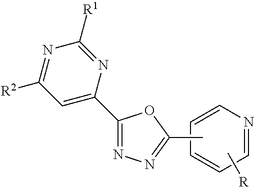

To a solution of the appropriate pyrimidine carboxylic acid (1 eq.) and DIPEA (3 eq.) in DMF is added the appropriate pyridine carboxylic acid hydrazide (2 eq.) followed by TBTU (1.5 eq.). The mixture is stirred for 3 to 12 h at rt. Formation of the hydrazide bond is monitored by LC-MS. The reaction mixture is then diluted with ethylacetate, washed with NaHCO₃, followed by brine, dried over Na₂SO₄, filtered and evaporated. The residue is dissolved in DCM. Pyridine (5 eq.) is added and the reaction mixture is cooled down to 0° C. before adding TFAA (1.1 eq.) and is then stirred at rt. Cyclization to the oxadiazole is monitored by LC-MS. Water is added to the mixture, the org. phase is collected, dried over Na₂SO₄, filtered and evaporated and the crude product is purified by either CC on silica gel, chromatography on prep. TLC plates, or by HPLC to give the desired 2-pyrimidin-4-yl-[1,3,4]oxadiazole derivatives.

### Examples 99 and 100

Using the above procedure, the following 2-pyrimidin-4-yl-[1,3,4]oxadiazole derivatives are prepared:

| Ex. | Pyrimidine | Pyridine | LC-MS* | | Amount |
|---|---|---|---|---|---|
| No. | (as acid) | (as hydrazide) | t_{R} [min] | [M + H]⁺ | Form |
| 99 | 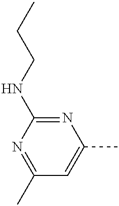 | 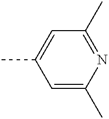 | 0.86 | 325.28 | 2.0 mg yellow powder |
| 100 | 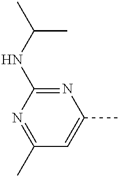 | 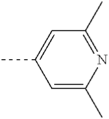 | 0.86 | 325.27 | 5.0 mg yellow powder |

### Example 99

¹H NMR (CDCl₃): δ 1.05 (t, J=7.3 Hz, 3H), 1.71 (m, 2H), 2.49 (s, 3H), 2.67 (s, 6H), 3.52 (m, 2H), 5.33 (s br, 1H), 7.31 (s, 1H), 7.73 (s, 2H).

### Example 100

¹H NMR (CDCl₃): δ 1.31 (d, J=6.5 Hz, 6H), 2.48 (s, 3H), 2.67 (s, 6H), 4.32 (m, 1H), 5.20 (d, J=6.8 Hz, 1H), 7.30 (s, 1H), 7.73 (s, 2H).

### Examples 101 to 137

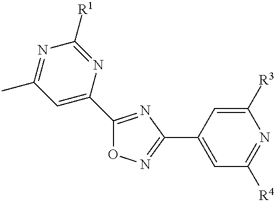

Following the general method for the preparation of 5-pyrimidin-4-yl-[1,2,4]oxadiazole derivatives, the following examples are prepared:

| Ex. | | Pyridine | LC-MS | | Amount |
|---|---|---|---|---|---|
| No. | R¹ | (as hydroxyamidine) | t_{R} [min] | [M + H]⁺ | Form |
| 101 | 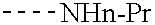 | 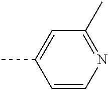 | 0.58 | 311.16 | 6.6 mg |
| 102 | 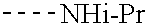 | 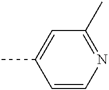 | 0.58 | 311.18 | 6.7 mg |
| 103 | 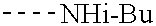 | 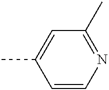 | 0.64 | 325.18 | 5.8 mg |
| 104 | 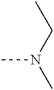 | 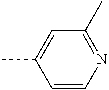 | 0.68 | 311.18 | 7.9 mg |
| 105 | 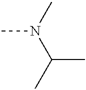 | 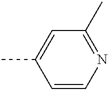 | 0.75 | 325.18 | 9.9 mg |
| 106 | 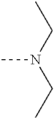 | 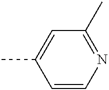 | 0.78 | 325.16 | 8.9 mg |
| 107 | 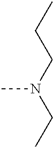 | 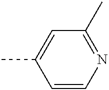 | 0.85 | 339.19 | 7.8 mg |
| 108 | 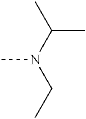 | 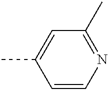 | 0.84 | 339.21 | 7.4 mg |
| 109 | 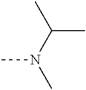 | 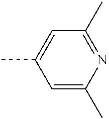 | 0.68 | 339.17 | 11.3 mg |
| 110 | 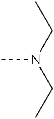 | 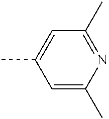 | 0.70 | 339.18 | 10.3 mg |
| 111 | 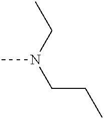 | 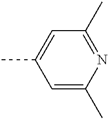 | 0.76 | 353.22 | 11.6 mg |
| 112 | 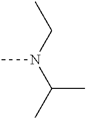 | 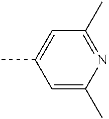 | 0.76 | 353.23 | 64 mg |
| 113 | 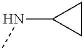 | 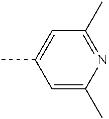 | 0.72 | 322.98 | 49 mg |
| 114 | 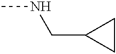 | 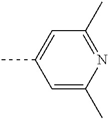 | 0.83 | 337.38 | 26.5 mg yellow solid |
| 115 | 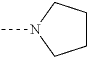 | 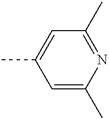 | 0.84 | 337.27 | 19 mg yellow solid |
| 116 | 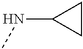 | 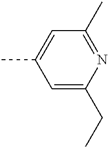 | 0.80 | 337.44 | 17 mg yellow solid |
| 117 | 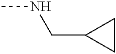 | 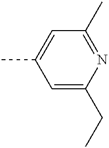 | 0.86 | 351.06 | 85 mg yellow solid |
| 118 | 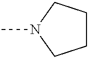 | 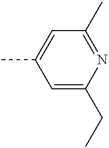 | 0.87 | 351.11 | 12 mg beige solid |
| 119 | 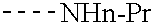 | 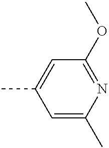 | 1.09 | 341.09 | 16.7 mg |
| 120 | 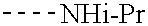 | 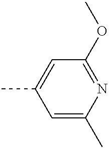 | 1.09 | 341.11 | 16.5 mg |
| 121 | 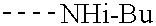 | 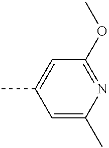 | 1.13 | 355.11 | 16.9 mg |
| 122 | 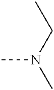 | 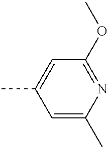 | 1.20 | 341.07 | 15.7 mg |
| 123 | 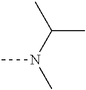 | 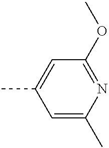 | 1.24 | 355.09 | 11.8 mg |
| 124 | 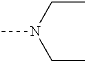 | 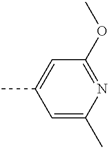 | 1.27 | 355.13 | 17.2 mg |
| 125 | 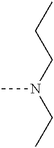 | 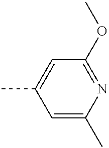 | 1.35 | 369.15 | 20.6 mg |
| 126 | 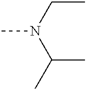 | 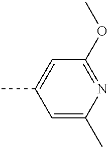 | 1.34 | 369.05 | 19.6 mg |
| 127 | 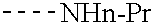 | 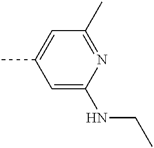 | 0.83 | 354.08 | 18 mg yellow solid |
| 128 | 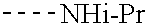 | 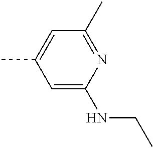 | 0.82 | 354.04 | 32.9 mg pale yellow solid |
| 129 | 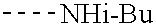 | 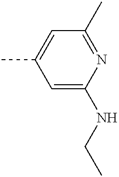 | 0.86 | 368.09 | 30.3 mg yellow solid |
| 130 | 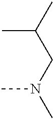 | 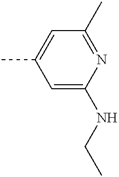 | 0.93 | 382.11 | 45.5 mg yellow solid |
| 131 | 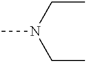 | 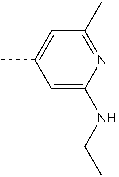 | 0.92 | 368.10 | 56.9 mg yellow solid |
| 132 | 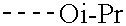 | 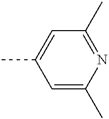 | 0.78 | 325.94 | 46 mg white solid |
| 133 | 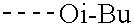 | 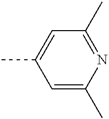 | 0.83 | 340.05 | 36 mg white solid |
| 134 | 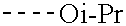 | 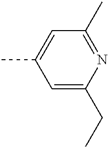 | 0.82 | 339.98 | 25 mg white solid |
| 135 | 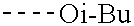 | 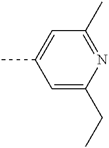 | 0.87 | 354.01 | 44 mg colorless wax |
| 136 | 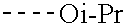 | 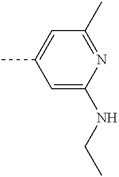 | 0.82 | 355.04 | 36 mg yellow solid |
| 137 | 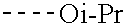 | 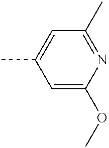 | 1.11 | 341.98 | 52 mg white solid |

### Example 113

¹H NMR (CDCl₃): δ 0.58 (m, 2H), 0.88 (m, 2H), 2.52 (s, 3H), 2.63 (s, 6H), 2.93 (m, 1H), 5.62 (s, 1H), 7.35 (s, 1H), 7.71 (s, 2H).

### Example 117

¹H NMR (CDCl₃): δ 0.32 (m, 2H), 0.58 (m, 2H), 1.13 (m, 1H), 1.37 (t, J=7.5 Hz, 3H), 2.51 (m, 3H), 2.66 (s, 3H), 2.92 (q, J=7.5 Hz, 2H), 3.41 (t, J=6.3 Hz, 2H), 5.56 (s br, 1H), 7.33 (s, 1H), 7.76 (s, 2H).

### Example 131

¹H NMR (CDCl₃): δ 1.24 (t, J=7.0 Hz, 6H), 1.30 (t, J=7.3 Hz, 3H), 2.46 (s, 3H), 2.47 (s, 3H), 3.38 (m, 2H), 3.74 (q, J=7.0 Hz, 4H), 4.68 (t, J=5.3 Hz, 1H), 6.94 (s, 1H), 7.19 (s, 1H), 7.21 (s, 1H).

### Example 132

¹H NMR (CDCl₃): δ 1.47 (d, J=6.3 Hz, 6H), 2.63 (s, 3H), 2.66 (s, 6H), 5.48 (m, 1H), 7.70 (s, 1H), 7.75 (s, 2H).

### Example 136

¹H NMR (CDCl₃): δ 1.31 (t, J=7.0 Hz, 3H), 1.46 (d, J=6.0 Hz, 6H), 2.47 (s, 3H), 2.62 (s, 3H), 3.38 (m, 2H), 4.68 (t, J=5.0 Hz, 1H), 5.48 (m, 1H), 6.94 (s, 1H), 7.19 (s, 1H), 7.69 (s, 1H).

### Example 137

¹H NMR (CDCl₃): δ 1.48 (d, J=0.5 Hz, 6H), 2.58 (s, 3H), 2.63 (s, 3H), 4.02 (s, 3H), 5.48 (m, 1H), 7.33 (d, J=0.5 Hz, 1H), 7.50 (s, 1H), 7.70 (s, 1H).

### Examples 138 to 150

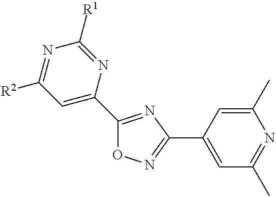

Following the general method for the preparation of 5-pyrimidin-4-yl-[1,2,4]oxadiazole derivatives, the following examples are prepared:

| Ex. | | | LC-MS | | Amount |
|---|---|---|---|---|---|
| No. | R¹ | R² | t_{R} [min] | [M + H]⁺ | Form |
| 138 | 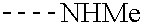 | 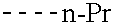 | 0.77 | 325.92 | 26 mg white solid |
| 139 | 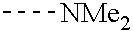 | 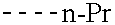 | 0.88 | 339.01 | 42 mg yellow solid |
| 140 | 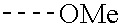 | 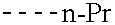 | 0.78 | 325.98 | 43 mg yellow solid |
| 141 | 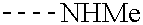 | 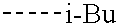 | 0.81 | 339.07 | 13 mg white solid |
| 142 | 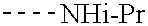 | 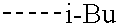 | 0.89 | 367.10 | 29 mg white solid |
| 143 | 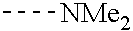 | 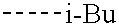 | 0.92 | 353.08 | 27 mg yellow solid |
| 144 | 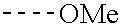 | 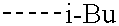 | 0.82 | 340.05 | 25 mg yellow solid |
| 145 | 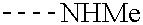 | 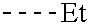 | 0.73 | 310.97 | 1.2 mg pale yellow oil |
| 146 | 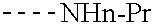 | 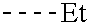 | 0.82 | 339.04 | 3.5 mg yellow oil |
| 147 | 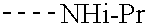 | 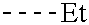 | 0.83 | 339.05 | 4.5 mg yellow solid |
| 148 | 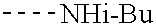 | 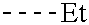 | 0.87 | 353.10 | 3.5 mg yellow solid |
| 149 | 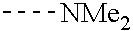 | 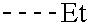 | 0.84 | 324.97 | 13 mg yellow oil |
| 150 | 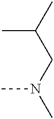 | 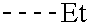 | 0.96 | 367.10 | 1.5 mg yellow oil |

### Example 139

¹H NMR (CDCl₃): δ 1.04 (t, J=7.3 Hz, 3H), 1.84 (m, 2H), 2.66-2.74 (m, 8H), 3.32 (s, 6H), 7.24 (s, 1H), 7.77 (s, 2H).

### Example 140

¹H NMR (CDCl₃): δ 1.05 (t, J=7.3 Hz, 3H), 1.88 (m, 2H), 2.67 (s, 6H), 2.86 (m, 2H), 4.17 (s, 3H), 7.73 (s, 1H), 7.76 (s, 2H).

### Example 144

¹H NMR (CDCl₃): δ 1.03 (d, J=6.8 Hz, 6H), 2.28 (m, 1H), 2.67 (s, 6H), 2.75 (d, J=7.3 Hz, 2H), 4.17 (s, 3H), 7.71 (s, 1H), 7.76 (s, 2H).

### Example 149

¹H NMR (CDCl₃): δ 1.36 (t, J=7.5 Hz, 3H), 2.66 (s, 6H), 2.77 (q, J=7.5 Hz, 2H), 3.32 (s, 6H), 7.25 (s, 1H), 7.75 (s, 2H).

### Examples 151 to 159

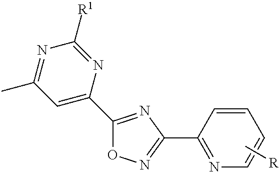

Following the general method for the preparation of 5-pyrimidin-4-yl-[1,2,4]oxadiazole derivatives, the following examples are prepared:

| | | | LC-MS | | Amount |
|---|---|---|---|---|---|
| Ex. No. | R¹ | 2-Pyridine | t_{R} [min] | [M + H]⁺ | Form |
| 151 | —NHEt | 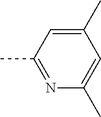 | 0.90 | 310.98 | 18 mg pale yellow solid |
| 152 | —NHi-Pr | 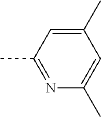 | 0.95 | 324.99 | 15 mg light brown solid |
| 153 | —NHEt | 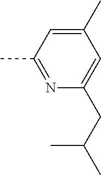 | 1.05 | 353.08 | 18 mg pale yellow solid |
| 154 | —NHi-Pr | 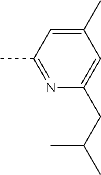 | 1.09 | 367.07 | 12.5 mg yellow oil |
| 155 | —NHi-Pr | 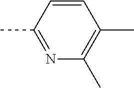 | 0.98 | 324.99 | 17 mg beige solid |
| 156 | —NHi-Pr | 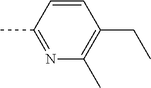 | 1.02 | 339.08 | 17 mg white solid |
| 157 | —NHi-Pr | 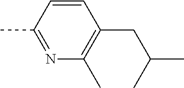 | 1.10 | 367.08 | 28 mg brown oil |
| 158 | —NHi-Pr | 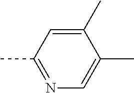 | 0.97 | 324.98 | 32 mg beige solid |
| 159 | —NHi-Pr | 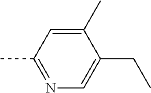 | 1.01 | 339.04 | 10 mg beige solid |

### Example 151

¹H NMR (CDCl₃): δ 1.28 (t, J=7.3 Hz, 3H), 2.42 (s, 3H), 2.47 (s, 3H), 2.66 (s, 3H), 3.58 (m, 2H), 5.58 (m, 1H), 7.15 (s, 1H), 7.38 (s, 1H), 7.90 (s, 1H).

### Example 153

¹H NMR (CDCl₃): δ 0.97 (d, J=6.5 Hz, 6H), 1.28 (t, J=7.0 Hz, 3H), 2.21 (m, 1H), 2.43 (s, 3H), 2.48 (s, 3H), 2.77 (d, J=7.3 Hz, 2H), 3.57 (m, 2H), 5.44 (t, J=5.0 Hz, 1H), 7.10 (s, 1H), 7.37 (s, 1H), 7.91 (s, 1H).

### Example 156

¹H NMR (CDCl₃): δ 1.30 (m, 9H), 2.48 (s, 3H), 2.69 (s, 3H), 2.75 (q, J=7.5 Hz, 2H), 4.32 (m, 1H), 5.25 (m, 1H), 7.38 (s, 1H), 7.63 (d, J=7.8 Hz, 1H), 8.04 (d, J=7.8 Hz, 1H).

### Examples 160 to 205

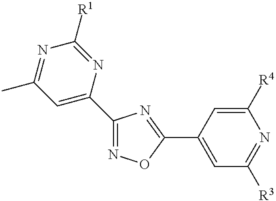

Following Method B for the preparation of 3-pyrimidin-4-yl-[1,2,4]oxadiazole derivatives, the following examples are prepared:

| | | Pyridine | LC-MS | | |
|---|---|---|---|---|---|
| Ex. No. | R¹ | (as carboxylic acid) | t_{R} [min] | [M + H]⁺ | Amount Form |
| 160 | ----NHn-Pr | 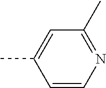 | 0.60 | 311.23 | 19 mg white solid |
| 161 | ----NHi-Pr | 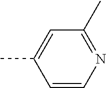 | 0.61 | 311.21 | 16 mg white solid |
| 162 | ----NHi-Bu | 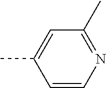 | 0.68 | 325.22 | 17 mg white solid |
| 163 | 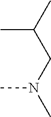 | 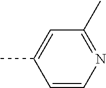 | 0.89 | 339.24 | 17 mg white solid |
| 164 | 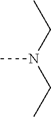 | 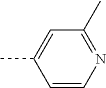 | 0.83 | 325.22 | 17 mg white solid |
| 165 | 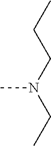 | 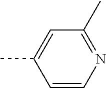 | 0.92 | 339.23 | 20 mg white solid |
| 166 | 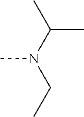 | 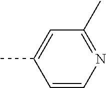 | 0.91 | 339.24 | 15 mg white solid |
| 167 | —NHMe | 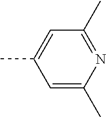 | 0.43 | 297.25 | 1.6 mg |
| 168 | —NHEt | 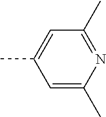 | 0.49 | 311.29 | 2.5 mg |
| 169 | ----NHn-Pr | 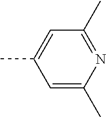 | 0.56 | 325.30 | 63 mg white solid |
| 170 | ----NHi-Pr | 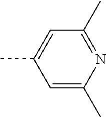 | 0.56 | 325.31 | 2.5 mg |
| 171 | ----NHi-Bu | 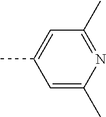 | 0.63 | 339.36 | 2.8 mg |
| 172 | —NMe₂ | 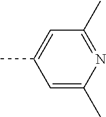 | 0.58 | 311.31 | 2.6 mg |
| 173 | 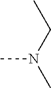 | 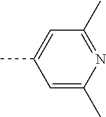 | 0.66 | 325.31 | 3.0 mg |
| 174 | 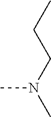 | 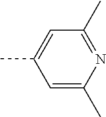 | 0.75 | 339.35 | 2.8 mg |
| 175 | 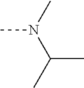 | 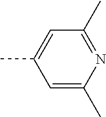 | 0.73 | 339.33 | 2.7 mg |
| 176 | 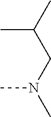 | 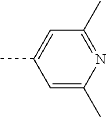 | 0.82 | 353.28 | 2.7 mg |
| 177 | 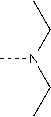 | 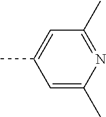 | 0.76 | 339.33 | 2.7 mg |
| 178 | 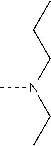 | 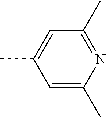 | 0.84 | 353.31 | 2.5 mg |
| 179 | 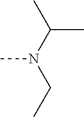 | 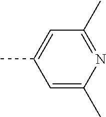 | 0.84 | 353.31 | 2.3 mg |
| 180 | ----OMe | 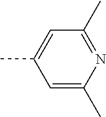 | 0.47 | 298.28 | 1.7 mg |
| 181 | ----OEt | 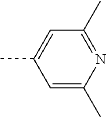 | 0.54 | 312.30 | 2.6 mg |
| 182 | ----On-Pr | 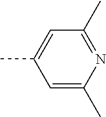 | 0.62 | 326.31 | 2.5 mg |
| 183 | ----Oi-Pr | 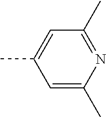 | 0.61 | 326.28 | 2.3 mg |
| 184 | ----Oi-Bu | 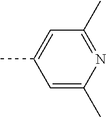 | 0.69 | 339.84 | 2.4 mg |
| 185 | ----NHn-Pr | 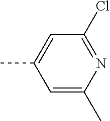 | 0.99 | 345.09 | 1.5 mg pale yellow solid |
| 186 | ----NH-Pr | 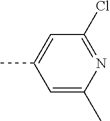 | 0.99 | 345.17 | 1.5 mg yellow wax |
| 187 | ----NHi-Bu | 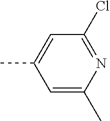 | 1.05 | 359.08 | 1.7 mg yellow wax |
| 188 | —NHMe | 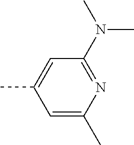 | 0.46 | 326.30 | 1.4 mg |
| 189 | —NHEt | 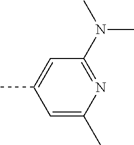 | 0.52 | 339.98 | 2.0 mg |
| 190 | ----NHn-Pr | 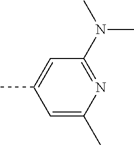 | 0.59 | 354.30 | 2.0 mg |
| 191 | ----NHi-Pr | 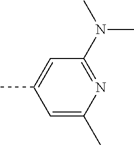 | 0.59 | 354.30 | 1.9 mg |
| 192 | ----NHi-Bu | 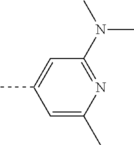 | 0.66 | 368.33 | 1.5 mg |
| 193 | ----NMe₂ | 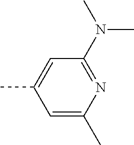 | 0.61 | 339.84 | 2 mg |
| 194 | 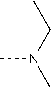 | 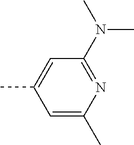 | 0.69 | 354.35 | 1.6 mg beige solid |
| 195 | 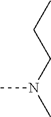 | 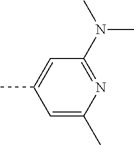 | 0.77 | 368.29 | 1.4 mg |
| 196 | 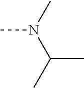 | 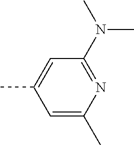 | 0.75 | 368.31 | 1.7 mg |
| 197 | 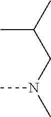 | 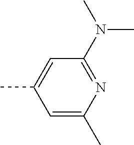 | 0.84 | 382.32 | 1.5 mg |
| 198 | 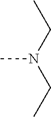 | 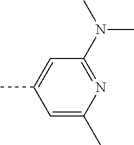 | 0.79 | 368.31 | 1.5 mg |
| 199 | 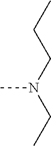 | 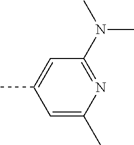 | 0.87 | 382.32 | 1.4 mg |
| 200 | 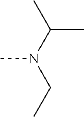 | 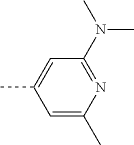 | 0.86 | 382.35 | 1.6 mg |
| 201 | ----OMe | 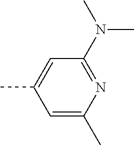 | 0.50 | 327.31 | 1.3 mg |
| 202 | ----OEt | 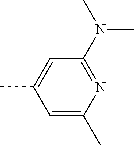 | 0.57 | 341.19 | 2.3 mg |
| 203 | ----On-Pr | 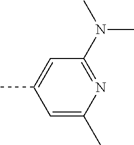 | 0.65 | 355.27 | 1.5 mg |
| 204 | ----Oi-Pr | 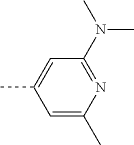 | 0.64 | 355.32 | 1.8 mg |
| 205 | ----Oi-Bu | 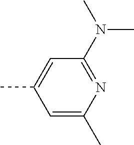 | 0.72 | 369.30 | 1.8 mg |

### Examples 206 to 237

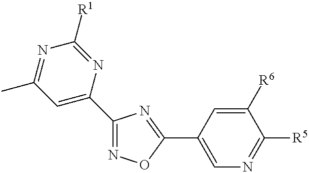

Following Method B for the preparation of 3-pyrimidin-4-yl-[1,2,4]oxadiazole derivatives, the following examples are prepared:

| | | 3-Pyridine | LC-MS | | Amount |
|---|---|---|---|---|---|
| Ex. No. | R¹ | (as carboxylic acid) | t_{R} [min] | [M + H]⁺ | Form |
| 206 | ----NHn-Pr | 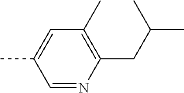 | 1.02 | 367.33 | 9.3 mg white solid |
| 207 | ----NHi-Pr | 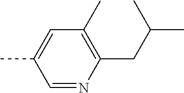 | 1.02 | 367.31 | 7.8 mg white solid |
| 208 | ----NHi-Bu | 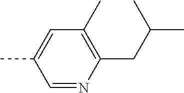 | 1.08 | 381.34 | 6.8 mg white solid |
| 209 | 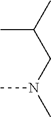 | 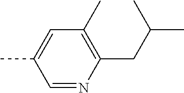 | 1.24 | 395.32 | 9.0 mg white solid |
| 210 | 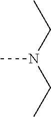 | 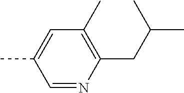 | 1.21 | 381.34 | 9.2 mg white solid |
| 211 | 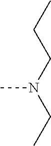 | 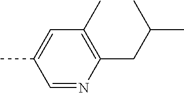 | 1.26 | 395.33 | 9.0 mg white solid |
| 212 | 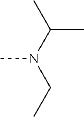 | 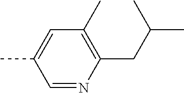 | 1.26 | 395.33 | 8.0 mg white solid |
| 213 | ----NHn-Pr | 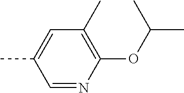 | 1.14 | 369.13 | 5.5 mg |
| 214 | ----NHi-Pr | 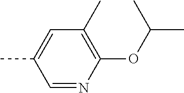 | 1.15 | 369.18 | 5.7 mg pale yellow solid |
| 215 | ----NHi-Bu | 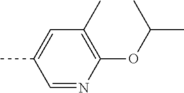 | 1.19 | 383.18 | 5.0 mg white solid |
| 216 | 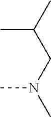 | 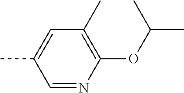 | 1.35 | 397.20 | 5.6 mg white solid |
| 217 | 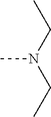 | 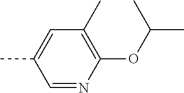 | 1.32 | 383.16 | 5.2 mg white solid |
| 218 | 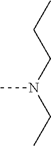 | 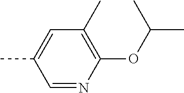 | 1.37 | 397.15 | 5.2 mg pale yellow solid |
| 219 | 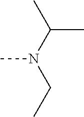 | 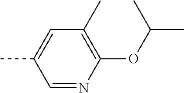 | 1.37 | 397.15 | 5.2 mg pale yellow solid |
| 220 | —NHMe | 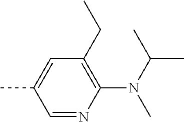 | 0.82 | 368.31 | 1 mg |
| 221 | —NHEt | 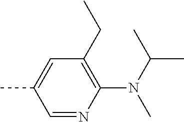 | 0.90 | 382.34 | 1 mg |
| 222 | ----NHn-pr | 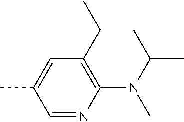 | 0.96 | 396.25 | 1 mg |
| 223 | ----NHi-Pr | 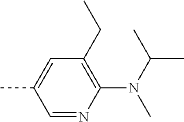 | 0.97 | 396.33 | 1 mg |
| 224 | ----NHi-Bu | 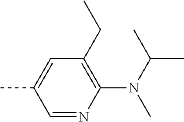 | 1.03 | 410.35 | 1 mg |
| 225 | —NMe₂ | 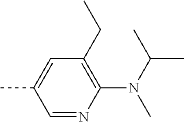 | 1.02 | 382.29 | 1.4 mg |
| 226 | 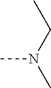 | 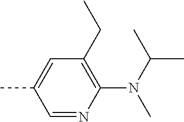 | 1.09 | 396.34 | 1.1 mg |
| 227 | 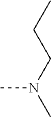 | 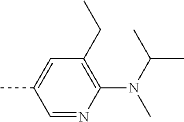 | 1.14 | 410.34 | 1.1 mg |
| 228 | 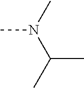 | 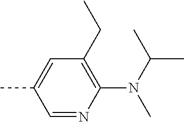 | 1.13 | 410.35 | 1.0 mg |
| 229 | 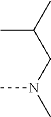 | 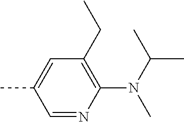 | 1.19 | 424.38 | 1.0 mg |
| 230 | 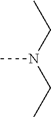 | 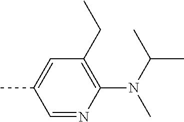 | 1.15 | 410.32 | 1.1 mg |
| 231 | 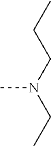 | 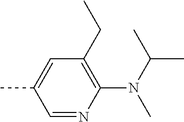 | 1.21 | 424.37 | 1.1 mg |
| 232 | 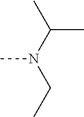 | 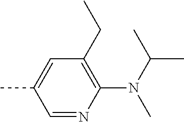 | 1.20 | 424.38 | 1.0 mg |
| 233 | ----OMe | 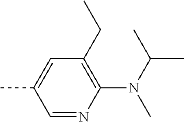 | 0.91 | 369.39 | 1.0 mg |
| 234 | ----OEt | 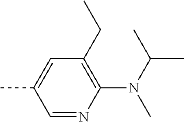 | 0.98 | 383.28 | 1.0 mg |
| 235 | ----On-Pr | 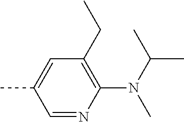 | 1.04 | 397.33 | 1.0 mg |
| 236 | ----Oi-Pr | 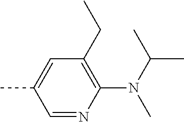 | 1.04 | 397.28 | 1.1 mg |
| 237 | ----Oi-Bu | 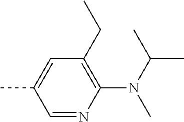 | 1.09 | 411.36 | 1.1 mg |

### Examples 238 to 255

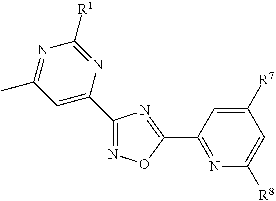

Following Method B for the preparation of 3-pyrimidin-4-yl-[1,2,4]oxadiazole derivatives, the following examples are prepared:

| | | | LC-MS | | A- |
|---|---|---|---|---|---|
| Ex. | | 2-Pyridine | t_{R} | [M + | |
| No. | R¹ | (as carboxylic acid) | [min] | H]⁺ | mount |
| 238 | —NHMe | 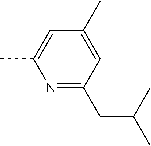 | 0.82 | 339.34 | 5.3 mg |
| 239 | —NHEt | 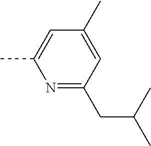 | 0.89 | 353.35 | 5.6 mg |
| 240 | ----NHn-Pr | 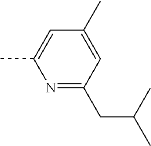 | 0.95 | 367.28 | 6.1 mg |
| 241 | ----NHi-Pr | 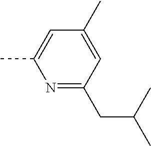 | 0.96 | 367.30 | 4.0 mg |
| 242 | ----NHi-Bu | 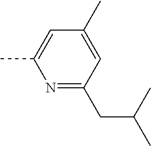 | 1.01 | 381.32 | 6.1 mg |
| 243 | —NMe2 | 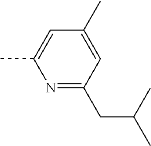 | 1.01 | 353.33 | 6.5 mg |
| 244 | 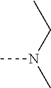 | 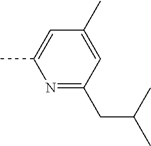 | 1.07 | 367.29 | 6.2 mg |
| 245 | 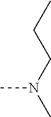 | 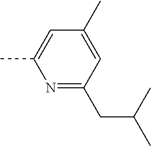 | 1.12 | 381.33 | 7.1 mg |
| 246 | 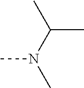 | 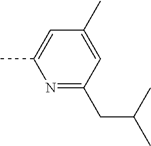 | 1.11 | 381.33 | 6.2 mg |
| 247 | 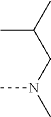 | 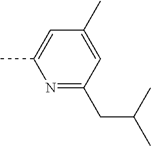 | 1.16 | 395.35 | 5.7 mg |
| 248 | 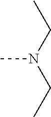 | 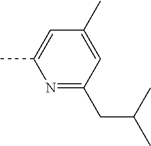 | 1.13 | 381.36 | 7.2 mg |
| 249 | 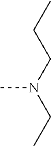 | 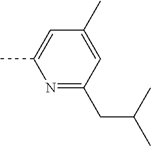 | 1.18 | 395.34 | 5.7 mg |
| 250 | 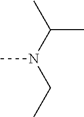 | 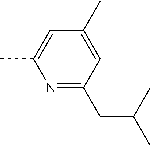 | 1.17 | 395.35 | 3.2 mg |
| 251 | ----OMe | 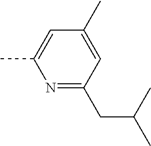 | 0.90 | 339.82 | 1.0 mg |
| 252 | ----OEt | 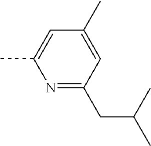 | 0.96 | 354.27 | 6.4 mg |
| 253 | ----On-Pr | 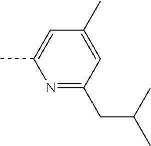 | 1.02 | 368.28 | 6.9 mg |
| 254 | ----Oi-Pr | 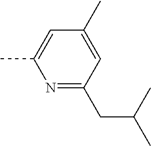 | 1.02 | 368.30 | 6.5 mg |
| 255 | ----Oi-Bu | 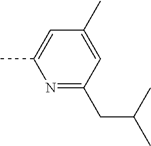 | 1.07 | 382.30 | 7.4 mg |

### Examples 256 to 260

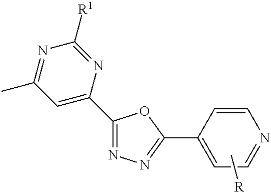

Following the general method for the preparation of 2-pyrimidin-4-yl-[1,3,4]oxadiazole derivatives, the following examples are prepared:

| | | | LC-MS | | |
|---|---|---|---|---|---|
| Ex. No. | R¹ | Pyridine | tR [min] | [M + H]⁺ | Amount Form |
| 256 | —NMe₂ | 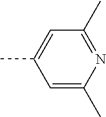 | 0.73 | 310.98 | 33 mg |

| | | | | | |
|---|---|---|---|---|---|
| 257 | 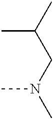 | 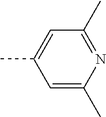 | 0.90 | 369.08 | 37 mg |
| 258 | 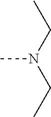 | 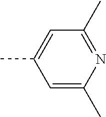 | 0.84 | 339.06 | 91 mg yellow solid |
| 259 | —NMe₂ | 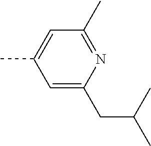 | 0.84 | 353.07 | 36 mg |
| 260 | ----Oi-Pr | 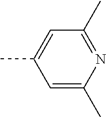 | 0.75 | 325.97 | 18 mg |

### Example 256

¹H NMR (CDCl₃): δ 2.49 (s, 3H), 2.68 (s, 6H), 3.32 (s, 6H), 7.25 (s, 1H), 7.72 (s, 2H).

### Example 259

¹H NMR (CDCl₃): δ 0.99 (d, J=6.5 Hz, 6H), 2.19 (m, 1H), 2.49 (s, 3H), 2.68 (s, 3H), 2.76 (d, J=7.3 Hz, 2H), 3.32 (s, 6H), 7.25 (s, 1H), 7.67 (s, 1H), 7.72 (s, 1H).

### General Method for the Preparation of 2-pyrimidin-4-yl-[1,3,4]thiadiazole derivatives

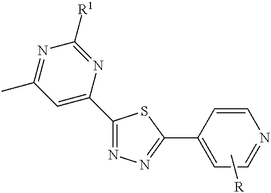

To a solution of the appropriate pyrimidine carboxylic acid (1 eq.) and DIPEA (3 eq.) in DMF is added the appropriate pyridine carboxylic acid hydrazide (2 eq.) followed by TBTU (1.5 eq.). The mixture is stirred for 3 to 12 h at rt. Formation of the hydrazide bond is monitored by LC-MS. The reaction mixture is then diluted with ethylacetate, washed with NaHCO₃, followed by brine, dried over Na₂SO₄, filtered and evaporated. The residue is dissolved in THF. Lawesson reagent (2 eq.) is added and the reaction mixture is heated at 110° C. under microwave conditions for 40 min. Cyclization to the thiadiazole is monitored by LC-MS. The reaction mixture is diluted with DCM and the organic solution is washed with sat. aq. NaHCO₃, dried over Na₂SO₄, filtered and evaporated and the crude product is purified by either CC on silica gel, chromatography on prep. TLC plates, or by HPLC to give the desired 5-pyrimidin-4-yl-[1,3,4]thiadiazole derivatives in 6 to 26% yield.

### Examples 261 to 265

Following the general method for the preparation of 5-pyrimidin-4-yl-[1,3,4]thiadiazole derivatives, the following examples are prepared:

| Ex. | | | LC-MS | | Amount |
|---|---|---|---|---|---|
| No. | R¹ | Pyridine | tR [min] | [M + H]⁺ | Form |
| 261 | ----NHn-Pr | 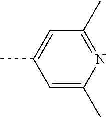 | 0.76 | 340.96 | 18 mg yellow solid |
| 262 | ----NH i-Pr | 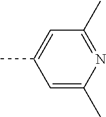 | 0.75 | 340.99 | 56 mg |
| 263 | —NHi-Bu | 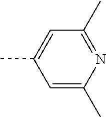 | 0.80 | 355.01 | 40 mg yellow solid |
| 264 | 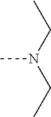 | 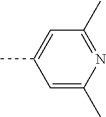 | 0.87 | 355.01 | 11 mg yellow solid |
| 265 | ----Oi-Pr | 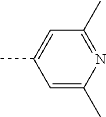 | 0.79 | 341.99 | 8.4 mg |

### Example 263

¹H NMR (CDCl₃): δ 1.04 (d, J=6.8 Hz, 6H), 1.97 (m, 1H), 2.48 (s, 3H), 2.66 (s, 6H), 3.35 (t, J=6.3 Hz, 2H), 5.36 (m, 1H), 7.44 (s, 1H), 7.61 (s, 2H).

### Example 265

¹H NMR (CD₃OD): δ1.46 (d, J=6.3 Hz, 6H), 2.59 (s, 3H), 2.62 (s, 6H), 5.39 (m, 1H), 7.74 (s, 2H), 7.81 (s, 1H).

### Examples 266 to 268

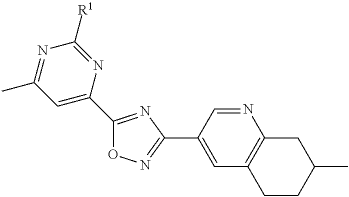

Following the general method for the preparation of 5-pyrimidin-4-yl-[1,3,4]oxadiazole derivatives, the following examples are prepared:

| | | LC-MS | | Amount |
|---|---|---|---|---|
| Ex. No. | R¹ | t_{R} [min] | [M + H]⁺ | Form |
| 266 | ----NHi-Pr | 0.95 | 367.12 | 123 mg beige glassy solid |
| 267 | 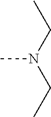 | 1.06 | 381.12 | 89 mg beige solid |
| 268 | ----Oi-Pr | 0.97 | 368.12 | 96 mg beige glassy solid |

### Example 266

¹H NMR (CDCl₃): δ 1.00 (d, J=6.8 Hz, 6H), 1.30 (d, J=6.5 Hz, 6H), 2.24 (m, 1H), 2.44 (s, 3H), 2.50 (s, 3H), 2.79 (d, J=7.3 Hz, 2H), 4.32 (m, 1H), 5.31 (m, 1H), 7.31 (s, 1H), 8.23 (s, 1H), 9.18 (d, J=1.8 Hz, 1H).

### Example 269

### GTPγS Assay to Determine EC₅₀ Values

GTPγS binding assays are performed in 96 well microtiter plates (Nunc, 442587) in a final volume of 200 μl, using membrane preparations of CHO cells expressing recombinant human S1P1 receptor. Assay conditions are 20 mM Hepes (Fluka, 54461), 100 mM NaCl (Fluka, 71378), 5 mM MgCl₂ (Fluka, 63064), 0.1% BSA (Calbiochem, 126609), 1 μM GDP (Sigma, G-7127), 2.5% DMSO (Fluka, 41644), 50 pM ³⁵S-GTPγS (Amersham Biosciences, SJ1320). The pH is 7.4. Test compounds are dissolved and diluted in 100% DMSO and pre-incubated at rt for 30 min in 150 μl of the above assay buffer, in the absence of ³⁵S-GTPγS. After addition of 50 μl of ³⁵S-GTPγS, the assay is incubated for 1 h at rt. The assay is terminated by transfer of the reaction mixture to a Multiscreen plate (Millipore, MAHFC1H60) using a cell harvester from Packard Biosciences, and the plates are washed with ice-cold 10 mM Na₂HPO₄/NaH₂PO₄ (70%/30%), dried, sealed at the bottom and, after addition of 25 μl MicroScint20 (Packard Biosciences, order# 6013621), sealed on the top. Membrane-bound ³⁵S-GTPγS is measured with a TopCount from Packard Biosciences.

EC₅₀ is the concentration of agonist inducing 50% of the maximal specific ³⁵S-GTPγS binding. Specific binding is determined by subtracting non-specific binding from maximal binding. Maximal binding is the amount of cpm bound to the Multiscreen plate in the presence of 10 μM of S1P. Non-specific binding is the amount of binding in the absence of an agonist in the assay.

Examples 58, 70, 78, 180, 224, 227, 229, 231, 232, 237, 240, 241, 242, 245, 246, 247, 248, 249, 250, 253, 254, 255 and 257 showed EC₅₀ values above 10 μM. EC₅₀ values of all other exemplified compounds are in the range of 0.5 to 8887 nM with an average of 1079 nM. Agonistic activities of some compounds of formula (I), determined according to the method described above, are displayed in Table 1.

TABLE 1

| Compound of Example | EC₅₀ [nM] |
|---|---|
| 9 | 21.0 |
| 12 | 27.0 |
| 21 | 1.6 |
| 25 | 46 |
| 27 | 8.0 |
| 51 | 7.8 |
| 60 | 126 |
| 79 | 1.2 |
| 81 | 30 |
| 94 | 27 |
| 100 | 128 |
| 101 | 11 |
| 109 | 1.3 |
| 116 | 2.1 |
| 118 | 186 |
| 119 | 30 |
| 126 | 39 |
| 132 | 44 |
| 135 | 142 |
| 138 | 9.8 |
| 142 | 26 |
| 147 | 0.7 |
| 149 | 63 |
| 155 | 20 |
| 159 | 53 |
| 162 | 5890 |
| 166 | 2150 |
| 168 | 38 |
| 174 | 34 |
| 177 | 11 |
| 178 | 11 |
| 182 | 395 |
| 185 | 22 |
| 187 | 31 |
| 188 | 560 |
| 196 | 241 |
| 200 | 94 |
| 201 | 1140 |
| 205 | 3040 |
| 206 | 5778 |
| 211 | 795 |
| 214 | 720 |
| 220 | 231 |
| 223 | 4160 |
| 228 | 3220 |
| 233 | 190 |
| 238 | 402 |
| 243 | 318 |
| 251 | 201 |
| 261 | 331 |
| 266 | 161 |
| 268 | 152 |

### Example 270

### Assessment of In Vivo Efficacy

The efficacy of the compounds of formula (I) is assessed by measuring the circulating lymphocytes after oral administration of 3 to 30 mg/kg of a compound of formula (I) to normotensive male Wistar rats. The animals are housed in climate-controlled conditions with a 12 h-light/dark cycle, and have free access to normal rat chow and drinking water. Blood is collected before and 3, 6 and 24 h after drug administration. Full blood is subjected to hematology using Advia Hematology system (Bayer Diagnostics, Zurich, Switzerland).

All data are presented as mean±SEM. Statistical analyses are performed by analysis of variance (ANOVA) using Statistica (StatSoft) and the Student-Newman-Keuls procedure for multiple comparisons. The null hypothesis is rejected when p<0.05.

As an example, Table 2 shows the effect on lymphocyte counts 6 h after oral administration of 10 mg/kg of some compounds of formula (I) to normotensive male Wistar rats as compared to a group of animals treated with vehicle only.

TABLE 2

| Compound of Example | Lymphocyte counts |
|---|---|
| 8 | −67 ± 2% |
| 15 | −69 ± 1% |
| 21 | −71 ± 2% |
| 51 | −67 ± 3% |
| 79 | −70 ± 2% |
| 110 | −67 ± 2% |
| 169 | −65 ± 3% |

1. A compound of the formula (I), 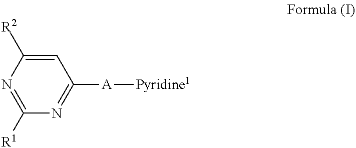
wherein
A represents 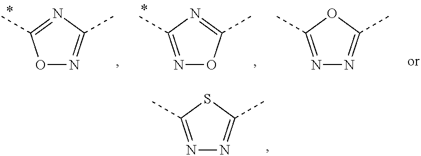
wherein the asterisks indicate the bond that is linked to the pyrimidine group of formula (I);
R¹ represents C₁₋₄-alkoxy, C₁₋₄-alkylamino, N—C₁₋₄-alkyl-N—C₁₋₃-alkylamino, C₃₋₅-cycloalkylamino, C₃₋₅-cycloalkylmethylamino, pyrrolidine, or piperidine;
R² represents C₁₋₂-alkyl, or C₃₋₄-alkyl; and
Pyridine¹ represents 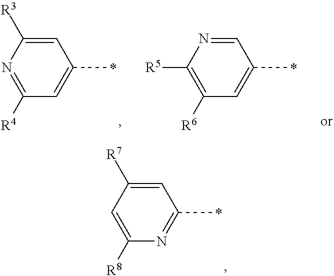
wherein the asterisks indicate the bond with which the pyridine ring is bound to the ring A of formula (I);
and in case R¹ represents C₁₋₄-alkylamino, N—C₁₋₄-alkyl-N—C₁₋₃-alkylamino, C₃₋₅-cycloalkylamino, or C₃₋₅-cycloalkylmethylamino, Pyridine¹ can also represent 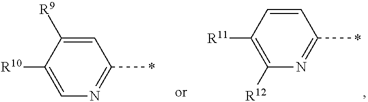
wherein the asterisks indicate the bond with which the pyridine ring is bound to the ring A of formula (I);
R³ represents C₁₋₄-alkyl, C₁₋₃-alkoxy, or NR^{3a}R^{3b};
R^{3a} represents C₁₋₂-alkyl;
R^{3b} represents hydrogen, or methyl; and
R⁴ represents hydrogen, chloro, or C₁₋₂alkyl;
R⁵ represents C₁₋₄-alkyl, C₁₋₃-alkoxy, or NR^{5a}R^{5b};
R^{5a} represents C₁₋₃-alkyl;
R^{5b} represents hydrogen, or C₁₋₂-alkyl; and
R⁶ represents C₁₋₂-alkyl;
R⁷ represents C₁₋₄-alkyl, and R⁸ represents C₁₋₂-alkyl; or
R⁷ represents C₁₋₂-alkyl, and R⁸ represents C₁₋₄-alkyl;
R⁹ represents C₁₋₂-alkyl; and
R¹⁰ represents C₁₋₄-alkyl;
and
R¹¹ represents C₁₋₄-alkyl; and
R¹² represents C₁₋₂-alkyl;
or a salt thereof.

2. A compound according to claim 1, Pyridine¹ represents 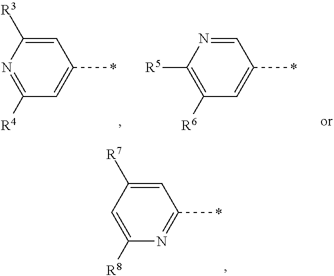
wherein the asterisks indicate the bond with which the pyridine ring is bound to the ring A of formula (I).

3. A compound according to claim 1, wherein A represents 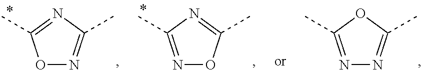
wherein the asterisks indicate the bond that is linked to the pyrimidine group of formula (I); or a salt thereof.

4. A compound according to claim 1, wherein A represents 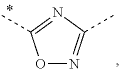
wherein the asterisk indicates the bond that is linked to the pyrimidine group of formula (I); or a salt thereof.

5. A compound according to claim 1, wherein R¹ represents C₁₋₄-alkylamino, or N—C₁₋₄-alkyl-N—C₁₋₃-alkylamino; or a salt thereof.

6. A compound according to claim 1, wherein R¹ represents C₁₋₄-alkylamino; or a salt thereof.

7. A compound according to claim 1, wherein R² represents C₁₋₂-alkyl; or a salt thereof.

8. A compound according to claim 1, wherein Pyridine¹ represents 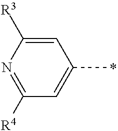
wherein the asterisk indicates the bond with which the pyridine ring is bound to the ring A of formula (I);
R³ represents C₁₋₄-alkyl, or NR^{3a}R^{3b};
R^{3a} represents C₁₋₂-alkyl;
R^{3b} represents hydrogen, or methyl; and
R⁴ represents methyl;
or a salt thereof.

9. A compound according to claim 1, wherein
A represents 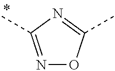
wherein the asterisk indicates the bond that is linked to the pyrimidine group of formula (I);
R¹ represents C₁₋₂-alkoxy, C₁₋₂-alkyl amino, or N-methyl-N—C₁₋₂-alkyl-amino; and
R² represents methyl;
or a salt thereof.

10. A compound according to claim 1, wherein
A represents 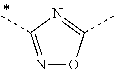
wherein the asterisk indicates the bond that is linked to the pyrimidine group of formula (I);
R¹ represents C₃₋₄-alkylamino, or N—C₃₋₄-alkyl-N—C₁₋₂-alkyl-amino; and
R² represents methyl;
or a salt thereof.

11. A compound according to claim 1, wherein
Pyridine¹ represents 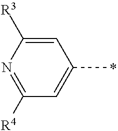
wherein the asterisk indicates the bond with which the pyridine ring is bound to the ring A of formula (I);
R³ represents C₃₋₄-alkyl; and
R⁴ represents C₁₋₂-alkyl;
or a salt thereof.

12. A compound according to claim 1, wherein
Pyridine¹ represents 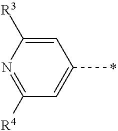
wherein the asterisk indicates the bond with which the pyridine ring is bound to the ring A of formula (I);
R³ represents CH₃; and
R⁴ represents C₁₋₂-alkyl;
or a salt thereof.

13. A compound according to claim 1, wherein
A represents 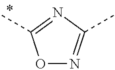
wherein the asterisk indicates the bond that is linked to the pyrimidine group of formula (I);
R¹ represents C₁₋₄-alkylamino, or N—C₁₋₄-alkyl-N—C₁₋₃-alkylamino;
R² represents C₁₋₂-alkyl; and
Pyridine¹ represents 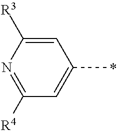
wherein the asterisk indicates the bond with which the pyridine ring is bound to the ring A of formula (I);
R³ represents methyl, ethyl, isopropyl, methylamino, or dimethylamino; and
R⁴ represents methyl, or chloro;
or a salt thereof.

14. A compound according to claim 1 selected from the group consisting of:
{4-[3-(2,6-dimethyl-pyridin-4-yl)-[1,2,4]oxadiazol-5-yl]-6-methyl-pyrimidin-2-yl}-ethyl-amine;
ethyl-{4-[3-(2-ethyl-6-methyl-pyridin-4-yl)-[1,2,4]oxadiazol-5-yl]-6-methyl-pyrimidin-2-yl}-amine;
{4-[3-(2,6-dimethyl-pyridin-4-yl)-[1,2,4]oxadiazol-5-yl]-6-methyl-pyrimidin-2-yl}-propyl-amine;
{4-[3-(2-ethyl-6-methyl-pyridin-4-yl)-[1,2,4]oxadiazol-5-yl]-6-methyl-pyrimidin-2-yl}-propyl-amine;
{4-[3-(2,6-dimethyl-pyridin-4-yl)-[1,2,4]oxadiazol-5-yl]-6-methyl-pyrimidin-2-yl}-isopropyl-amine;
{4-[3-(2-ethyl-6-methyl-pyridin-4-yl)-[1,2,4]oxadiazol-5-yl]-6-methyl-pyrimidin-2-yl}-isopropyl-amine;
{4-[3-(2,6-dimethyl-pyridin-4-yl)-[1,2,4]oxadiazol-5-yl]-6-methyl-pyrimidin-2-yl}-isobutyl-amine;
{4-[3-(2,6-dimethyl-pyridin-4-yl)-[1,2,4]oxadiazol-5-yl]-6-methyl-pyrimidin-2-yl}-ethyl-methyl-amine;
{4-[3-(2,6-dimethyl-pyridin-4-yl)-[1,2,4]oxadiazol-5-yl]-6-methyl-pyrimidin-2-yl}-methyl-propyl-amine;
{4-[3-(2,6-dimethyl-pyridin-4-yl)-[1,2,4]oxadiazol-5-yl]-6-methyl-pyrimidin-2-yl}-isobutyl-methyl-amine;
{4-[3-(2,6-dimethyl-pyridin-4-yl)-[1,2,4]oxadiazol-5-yl]-6-ethyl-pyrimidin-2-yl}-ethyl-amine;
{4-[5-(2-Isobutyl-6-methyl-pyridin-4-yl)-[1,2,4]oxadiazol-3-yl]-6-methyl-pyrimidin-2-yl}-dimethyl-amine; and
{4-[5-(2-isobutyl-6-methyl-pyridin-4-yl)-[1,2,4]oxadiazol-3-yl]-6-methoxy-6-methyl-pyrimidine;
or a salt of such compounds.

15. A compound according to claim selected from the group consisting of:
isopropyl-{4-methyl-6-[3-(2-methyl-pyridin-4-yl)-[1,2,4]oxadiazol-5-yl]-pyrimidin-2-yl}-amine;
{4-[3-(2,6-dimethyl-pyridin-4-yl)-[1,2,4]oxadiazol-5-yl]-6-methyl-pyrimidin-2-yl}-isopropyl-methyl-amine;
{4-[3-(2,6-dimethyl-pyridin-4-yl)-[1,2,4]oxadiazol-5-yl]-6-methyl-pyrimidin-2-yl}-diethyl-amine;
{4-[3-(2,6-dimethyl-pyridin-4-yl)-[1,2,4]oxadiazol-5-yl]-6-methyl-pyrimidin-2-yl}-ethyl-propyl-amine;
{4-[3-(2,6-dimethyl-pyridin-4-yl)-[1,2,4]oxadiazol-5-yl]-6-methyl-pyrimidin-2-yl}-ethyl-isopropyl-amine;
cyclopropyl-{4-[3-(2,6-dimethyl-pyridin-4-yl)-[1,2,4]oxadiazol-5-yl]-6-methyl-pyrimidin-2-yl}-amine;
cyclopropyl-{4-[3-(2-ethyl-6-methyl-pyridin-4-yl)-[1,2,4]oxadiazol-5-yl]-6-methyl-pyrimidin-2-yl}-amine;
isopropyl-{4-[3-(2-methoxy-6-methyl-pyridin-4-yl)-[1,2,4]oxadiazol-5-yl]-6-methyl-pyrimidin-2-yl}-amine;
4-[3-(2,6-dimethyl-pyridin-4-yl)-[1,2,4]oxadiazol-5-yl]-2-isopropoxy-6-methyl-pyrimidine;
4-[3-(2,6-dimethyl-pyridin-4-yl)-[1,2,4]oxadiazol-5-yl]-2-isobutoxy-6-methyl-pyrimidine;
{4-[3-(2,6-dimethyl-pyridin-4-yl)-[1,2,4]oxadiazol-5-yl]-6-propyl-pyrimidin-2-yl}-methyl-amine;
{4-[3-(2,6-dimethyl-pyridin-4-yl)-[1,2,4]oxadiazol-5-yl]-6-propyl-pyrimidin-2-yl}-dimethyl-amine;
{4-[3-(2,6-dimethyl-pyridin-4-yl)-[1,2,4]oxadiazol-5-yl]-6-ethyl-pyrimidin-2-yl}-propyl-amine;
{4-[3-(2,6-dimethyl-pyridin-4-yl)-[1,2,4]oxadiazol-5-yl]-6-ethyl-pyrimidin-2-yl}-isopropyl-amine;
{4-[3-(2,6-dimethyl-pyridin-4-yl)-[1,2,4]oxadiazol-5-yl]-6-ethyl-pyrimidin-2-yl}-isobutyl-amine;
{4-[3-(2,6-dimethyl-pyridin-4-yl)-[1,2,4]oxadiazol-5-yl]-6-ethyl-pyrimidin-2-yl}-isobutyl-methyl-amine;
{4-[5-(2,6-dimethyl-pyridin-4-yl)-[1,2,4]oxadiazol-3-yl]-6-methyl-pyrimidin-2-yl}-propyl-amine;
{4-[5-(2,6-dimethyl-pyridin-4-yl)-[1,2,4]oxadiazol-3-yl]-6-methyl-pyrimidin-2-yl}-isopropyl-amine;
{4-[5-(2,6-dimethyl-pyridin-4-yl)-[1,2,4]oxadiazol-3-yl]-6-methyl-pyrimidin-2-yl}-isobutyl-methyl-amine;
{4-[5-(2,6-dimethyl-pyridin-4-yl)-[1,2,4]oxadiazol-3-yl]-6-methyl-pyrimidin-2-yl}-diethyl-amine;
{4-[5-(2,6-dimethyl-pyridin-4-yl)-[1,2,4]oxadiazol-3-yl]-6-methyl-pyrimidin-2-yl}-ethyl-propyl-amine; and
{4-[5-(2,6-dimethyl-pyridin-4-yl)-[1,2,4]oxadiazol-3-yl]-6-methyl-pyrimidin-2-yl}-ethyl-isopropyl-amine;
or a salt of such compounds.

16. A pharmaceutical composition comprising a compound according to claim 1, or a pharmaceutically acceptable salt thereof, and a pharmaceutically acceptable carrier.

17. (canceled)

18. A method for the treatment or prophylaxis of diseases or disorders associated with an activated immune system comprising administering to a patient in need thereof an effective amount of a compound according to claim 1 or a pharmaceutically acceptable salt thereof.

19. A method according to claim 18 wherein said disease or disorder is selected from the group consisting of rejection of transplanted organs, kidney, liver, heart, lung, pancreas, cornea, and skin; graft-versus-host diseases brought about by stem cell transplantation; autoimmune syndromes, rheumatoid arthritis, multiple sclerosis, inflammatory bowel diseases, Crohn's disease and ulcerative colitis, psoriasis, psoriatic arthritis, thyroiditis, Hashimoto's thyroiditis, uveo-retinitis; atopic diseases, rhinitis, conjunctivitis, dermatitis; asthma; type I diabetes; post-infectious autoimmune diseases, rheumatic fever and post-infectious glomerulonephritis; solid cancers and tumor metastasis.
